(19) United States
(12) Patent Application Publication
(10) Pub. No.: US 2023/0339871 A1
(43) Pub. Date: Oct. 26, 2023

(54) COMPOUNDS AND METHODS OF MODULATING 17B-HYDROXYSTEROID DEHYDROGENASE TYPE 13

(71) Applicant: METREA BIOSCIENCES, INC., San Francisco, CA (US)

(72) Inventors: Paul Da Silva Jardine, Guilford, CT (US); Frederick Dewey, Winchester, MA (US); Joshua Hoffman, Needham Heights, MA (US)

(21) Appl. No.: 18/022,117

(22) PCT Filed: Aug. 18, 2021
(86) PCT No.: PCT/US2021/046519

§ 371 Date: Feb. 17, 2023

Related U.S. Application Data
(60) Provisional application No. 63/067,094, filed on Aug. 18, 2020.

(51) Int. Cl. C07D 277/04; C07D 417/06; A61K 31/4439; A61K 31/426; C07D 333/22; A61K 31/381; C07D 413/06; A61K 31/4178; C07D 215/48; C07D 401/12; A61K 31/4709; A61K 31/4704; C07D 263/06; C07D 217/26; A61K 31/47; A61P 1/16; A61K 31/4166; A61K 31/427; A61K 45/06

(52) U.S. Cl. CPC C07D 277/04; C07D 417/06; A61K 31/4439; A61K 31/426; C07D 333/22; A61K 31/381; C07D 413/06; A61K 31/4178; C07D 215/48; C07D 401/12; A61K 31/4709; A61K 31/4704; C07D 263/06; C07D 217/26; A61K 31/47; A61P 1/16; A61K 31/4166; A61K 31/427; A61K 45/06

(57) ABSTRACT

The present disclosure is generally directed to modulators of hydroxysteroid dehydrogenase enzymes useful in the treatment of diseases and disorders modulated by said enzyme and having the Formula (I), Formula (II), or Formula (III).

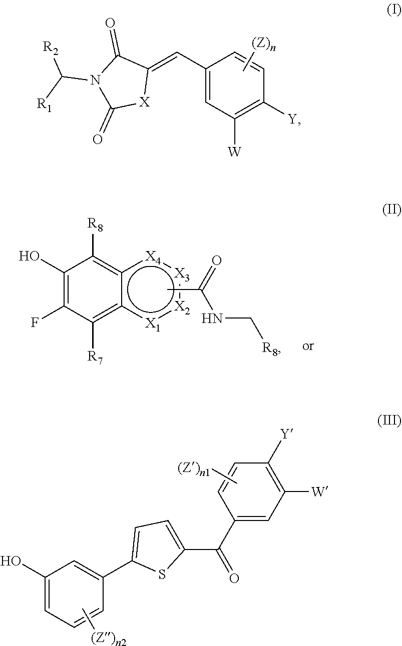

### CROSS-REFERENCE RELATED APPLICATION

This application claims the benefit of U.S. Provisional Patent Application No. 63/067,094, filed Aug. 18, 2020, entitled “COMPOUNDS AND METHODS OF MODULATING 170-HYDROXYSTEROID DEHYDROGENASE TYPE 13” which is incorporated herein by reference in its entirety.

### FIELD

The present disclosure is directed to modulators of the short-chain dehydrogenase/reductase (SDR) enzymes, which includes 17β-hydroxysteroid dehydrogenase type 13 (HSD17B13). The inhibitors described herein can be useful in the treatment of conditions associated with SDR enzymes. In particular, the disclosure is concerned with compounds and pharmaceutical compositions modulating HSD17B13, methods of treating conditions, such as fatty liver diseases, associated with HSD17B13, and methods of synthesizing these compounds BACKGROUND

Nonalcoholic fatty liver disease (NAFLD), defined as the presence of fat in the liver, encompasses a spectrum of fatty liver disorders that are currently estimated to reach a global prevalence of over 1 billion by 2030 In the United States alone, NAFLD is estimated to be the most common cause of chronic liver disease, affecting between 80 and 100 million individuals. As the rates of obesity and type 2 diabetes mellitus increase worldwide, the prevalence of NAFLD is increasing proportionately. To complicate the condition, NAFLD progresses at different rates among individuals and exhibits diverse clinical manifestations.

Nonalcoholic steatohepatitis (NASH) is a type of NAFLD and is defined by a buildup of fat in the liver (liver steatosis) associated with inflammation and ballooning of hepatocytes, with or without fibrosis. It is estimated that nearly 25% of patients with NAFLD progress to NASH. NASH is heavily influenced by lifestyle (e.g., chronic excessive calorie intake, sedentary activity) and is distinct from other fatty liver diseases caused by alcohol abuse or medication side effects. Many NASH patients are unaware of their liver condition and the buildup of fat, chronic hepatocellular ballooning and inflammation over time cause disease progression to more serious disease stages, such as advanced fibrosis, cirrhosis, liver failure, or liver cancer.

Despite its growing prevalence there are no efficacious therapies currently available for patients with advanced NASH and leading agents have exhibited low response rates. Numerous clinical studies for the treatment of NASH have failed to meet primary endpoints, including several late-stage clinical trials several of which involved promising combination therapies. Given the severity of NASH and the unmet clinical need, an effective therapeutic treatment is urgently needed.

### SUMMARY

A first aspect of the present disclosure relates to compounds of Formula I:

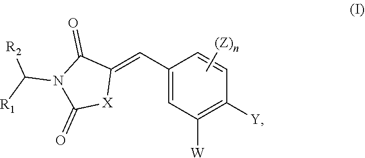

or a pharmaceutically acceptable salt, ester, solvate, amino acid conjugate, isomer, or tautomer thereof, wherein:
X is selected from O and S;
Y is selected from —H, —OH and —F;
W is —OH; or
W and Y, taken together with the atom to which they are each attached, form a 3- to 10-membered heterocycle or heteroaryl, wherein the heterocycle or heteroaryl is optionally substituted with one or more Z;
each Z is independently, at each occurrence, selected from halogen, —OH, —NH₂, —CN, C₁-C₄ alkyl, C₁-C₄ alkoxy, C₂-C₄ alkenyl, and C₂-C₄ alkynyl;
R₁ is independently selected from C₃-C₆ cycloalkyl, tetrahydropyranyl, tetrahydrofuranyl, pyridinyl, pyridonyl, piperidinyl, pyrrolidinyl, phenyl, and benzyl, wherein the phenyl is substituted with one or more R₃; and wherein the cycloalkyl, tetrahydropyranyl, tetrahydrofuranyl, pyridinyl, piperidinyl, pyridonyl, or pyrrolidinyl is optionally substituted with one or more R₄;
R₂ is selected from —H or —CH₃;
each R₃ is independently selected from halogen, —OH, C₁-C₆ alkoxy, —CF₃, haloalkoxy, —NO₂, —S(O)₂R₅, —NHSO₂C₁-C₄ alkyl, —NHCOC₁-C₄ alkyl, —CF₃, —COOH, —C(O)NH₂, C(O)NHR₅, S(O)₂NH₂, S(O)₂NHR₅, —CN, —C(O)OR₅, —NH₂, and heteroaryl;
each R₄ is independently selected from oxo, C₁-C₆ alkyl, C₁-C₄ alkoxy, —C(O)R₅, —CF₃, and —S(O)₂R₅;
each R₅ is independently selected from —H, C₁-C₆ alkyl, C₁-C₆ alkoxy, C₂-C₆ alkenyl, C₂-C₆ alkynyl, and —CF₃; and
n is an integer selected from 0, 1, and 2.

Another aspect of the present disclosure relates to compounds of Formula H:

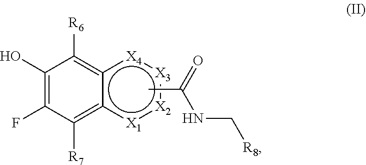

or a pharmaceutically acceptable salt, ester, solvate, amino acid conjugate, isomer, or tautomer thereof, wherein:
X₁ is selected from bond, C(R₁₀), and N;
X₂, X₃, and X₄ are independently selected from C(R₁₀), N, O, and S; provided that at least one of X₂, X₃, and X₄ is C(R₁₀) or N;
R₆ and R₇ are independently selected from —H, halogen, —NR₁₀R₁₁, —C(O)R₁₀, —C(O)NR₁₀R₁₁, —CN, C₁-C₆ alkyl, C₁-C₆ alkoxy, C₂-C₆ alkenyl, C₂-C₆ alkynyl, C₃-C₁₀ cycloalkyl, C₅-C₈ cycloalkenyl, C₅-C₈ spirocycloalkyl, spiroheterocyclyl, heterocyclyl, aryl, and heteroaryl, wherein the alkyl, alkoxy, alkenyl, alkynyl, cycloalkyl, cycloalkenyl, spirocycloalkyl, spiroheterocyclyl, heterocyclyl, aryl, or heteroaryl is optionally substituted with one or more R₉,
R₈ is selected from phenyl and heteroaryl, wherein the phenyl or heteroaryl is optionally substituted with one or more —OH, C₁-C₄ alkoxy, —S(O)₂—C₁-C₃ alkyl, —COOH, —CN, —CONH₂, or heteroaryl containing 1-4 heteroatoms selected from N, O, and S;
R₉, R₁₀, and R₁₁ are independently at each occurrence, selected from —H, halogen, C₁-C₆ alkyl, C₁-C₆ alkoxy, C₂-C₆ alkenyl, C₂-C₆ alkynyl, C₃-C₈ cycloalkyl, heterocyclyl, aryl, and heteroaryl.

Another aspect of the present disclosure relates to compounds of Formula III:

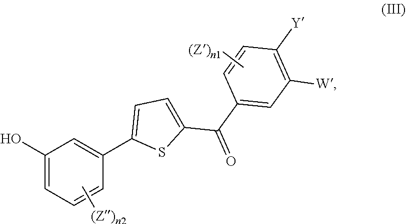

or a pharmaceutically acceptable salt, ester, solvate, amino acid conjugate, isomer, or tautomer thereof, wherein:
W′ is selected from —H, —OH, and —F;
Y′ is selected from —OH and —F;
each Z′ and Z″ are independently selected from halogen, —OH, —NH₂, —CN, C₁-C₄ alkyl, C₁-C₄ alkoxy, C₂-C₄ alkenyl, or C₂-C₄ alkynyl;
n1 is an integer selected from 0, 1, 2, or 3; and
n2 is an integer selected from 0, 1, 2, 3, or 4.

Another aspect of the present disclosure is directed to pharmaceutical compositions comprising a compound of Formula (I), Formula (II), or Formula (III), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the present disclosure relates to a method of treating a condition associated with modulation HSD17B13. The method comprises administering to a patient in need of a treatment for conditions associated with modulation of HSD17B13 an effective amount of a compound of Formula (I), Formula (II), or a pharmaceutically acceptable salt, hydrate, solvate, prodrug, stereoisomer, tautomer, or pharmaceutical composition thereof.

Another aspect of the present disclosure is directed to a method of inhibiting HSD17B13. The method involves administering to a patient in need thereof an effective amount of a compound of Formula (I), Formula (II), or Formula (III), or a pharmaceutically acceptable salt, hydrate, solvate, prodrug, stereoisomer, tautomer, or pharmaceutical composition thereof.

Another aspect of the present disclosure is directed to a method of treating or preventing a condition disclosed herein in a subject in need thereof. The method involves administering to a patient in need thereof an effective amount of a compound of Formula (I), Formula (II), or Formula (III), or a pharmaceutically acceptable salt, hydrate, solvate, prodrug, stereoisomer, tautomer, or pharmaceutical composition thereof.

Another aspect of the present disclosure relates to compounds of Formula (I), Formula (II), or Formula (III), and pharmaceutically acceptable salts, hydrates, solvates, prodrugs, stereoisomers, tautomers, or pharmaceutical compositions thereof, for use in the manufacture of a medicament for inhibiting HSD17B13.

Another aspect of the present disclosure relates to compounds of Formula (I), Formula (II), or Formula (III), and pharmaceutically acceptable salts, hydrates, solvates, prodrugs, stereoisomers, tautomers, or pharmaceutical compositions thereof, for use in the manufacture of a medicament for treating or preventing a condition disclosed herein.

Another aspect of the present disclosure relates to the use of a compound of Formula (I), Formula (U), or Formula (III), or a pharmaceutically acceptable salt, hydrate, solvate, prodrug, stereoisomer, tautomer, or pharmaceutical composition thereof, in the treatment of a disease associated with inhibiting HSD17B13.

Another aspect of the present disclosure relates to the use of a compound of Formula (I), Formula (II), or Formula (III), or a pharmaceutically acceptable salt, hydrate, solvate, prodrug, stereoisomer, tautomer, or pharmaceutical composition thereof, in the treatment of a condition disclosed herein.

The present disclosure further provides methods of treating a condition associated with modulation of HSD17B13, including primary sclerosing cholangitis, chronic liver disease, nonalcoholic fatty liver disease (NAFLD), nonalcoholic steatohepatitis (NASH), hepatitis C, alcoholic liver disease, liver damage due to progressive fibrosis, and/or liver fibrosis, comprising administering to a patient suffering from at least one of said conditions a compound of Formula (I), Formula (II), or Formula (III), or a pharmaceutically acceptable salt, hydrate, solvate, prodrug, stereoisomer, tautomer, or pharmaceutical composition thereof.

The present disclosure provides inhibitors of HSD17B13 that are therapeutic agents in the treatment of diseases such as primary sclerosing cholangitis, chronic liver disease, nonalcoholic fatty liver disease (NAFLD), nonalcoholic steatohepatitis (NASH), hepatitis C, alcoholic liver disease, liver damage due to progressive fibrosis, and liver fibrosis.

The present disclosure further provides compounds and compositions with an improved efficacy and safety profile relative to known HSD17B13 inhibitors. The present disclosure also provides agents with novel mechanisms of action toward HSD17B13 in the treatment of various types of diseases including primary sclerosing cholangitis, chronic liver disease, nonalcoholic fatty liver disease (NAFLD), nonalcoholic steatohepatitis (NASH), hepatitis C, alcoholic liver disease, liver damage due to progressive fibrosis, and liver fibrosis.

In some aspects, the present disclosure provides a compound obtainable by, or obtained by, a method for preparing compounds described herein (e.g., a method comprising one or more steps described in General Procedures I-V).

In some aspects, the present disclosure provides an intermediate as described herein, being suitable for use in a method for preparing a compound as described herein (e.g., the intermediate is selected from the intermediates described in Examples 1-46).

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed present disclosure. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE FIGURES

FIG. 1A is a chart demonstrating the anti-inflammatory effects of HSD17B13 inhibitors in HepG2 cells. HepG2 cells exposed to Compound 5 (3 mM, 10 mM, or 30 mM) showed decreased expression of IL-8 when exposed to a saturated fatty acid NASH challenge.

FIG. 1B is a chart demonstrating the anti-inflammatory effects of HSD17B13 inhibitors in HepG2 cells. HepG2 cells exposed to Compound 5 (3 mM, 10 mM, or 30 mM) showed decreased expression of IL-32 when exposed to a saturated fatty acid NASH challenge.

FIG. 2 is a chart illustrating triglyceride content following fatty acid stimulation in iPSC-derived hepatocytes.

FIG. 3 is a chart illustrating triglyceride content following fatty acid stimulation in iPSC-derived hepatocytes.

FIG. 4 is a chart illustrating de novo lipogenesis in iPSC-derived hepatocytes.

FIG. 5 is a chart illustrating de novo lipogenesis in iPSC-derived hepatocytes.

FIG. 6 is a chart demonstrating the association of the chronic fatty liver disease polygenic score with chronic fatty liver disease. Quantile 1 serves as the reference group.

FIG. 7 is a chart demonstrating the association of the chronic fatty liver disease polygenic score with cirrhosis. Quantile 1 serves as the reference group.

### DETAILED DESCRIPTION

The present disclosure relates to compounds and compositions that are capable of inhibiting the activity of HSD17B13. The disclosure features methods of treating, preventing or ameliorating a condition in which HSD17B13 plays a role by administering to a patient in need thereof a therapeutically effective amount of a compound of Formula (I), Formula (II), or Formula (III), or a pharmaceutically acceptable salt, hydrate, solvate, prodrug, stereoisomer, or tautomer thereof. The methods of the present disclosure can be used in the treatment of a variety of HSD17B13-mediated diseases and disorders by inhibiting the activity of HSD17B13. Inhibition of HSD17B13 can be an effective approach to the treatment, prevention, or amelioration of diseases including, but not limited to, primary sclerosing cholangitis, chronic liver disease, nonalcoholic fatty liver disease (NAFLD), nonalcoholic steatohepatitis (NASH), hepatitis C, alcoholic liver disease, liver damage due to progressive fibrosis, and liver fibrosis.

Nonalcoholic fatty liver disease (NAFLD) represents a spectrum of fatty liver abnormalities that has an increasing worldwide prevalence, in part due to the rise in diabetes and obesity, which can progress to more serious disease stages, such as advanced fibrosis, cirrhosis, liver failure, or liver cancer. Despite its growing prevalence there are no efficacious therapies currently available for patients with advanced NASH and leading agents have low response rates. Therapies have focused on numerous targets including those associated with the liver, gut, adipose tissue, and muscle as well as targets affecting the central nervous system by altering eating behavior or centrally regulated metabolism.

Numerous clinical studies for the treatment of NASH have failed to meet primary endpoints, including several late-stage clinical trials some of which involved combination therapies. Furthermore, clinical trials that were considered a success had agents that resulted in low response rates.

Phase 2 and 3 NASH clinical trials are summarized in Table 1 below.

TABLE 1
Summary of Major NASH Phase III Clinical Trials

| | | | | | Status |
|---|---|---|---|---|---|
| Entry | Agent | Company | Trial Name | Trial Number | (as of May 2020) |
| 1 | Selonsertib | Gilead | STELLAR 3 | NCT03053050 | Completed - failed in |
| | | | | | second Phase III trial |
| | | | | | (announced Q2 2019) |
| 2 | Selonsertib | Gilead | STELLAR 4 | NCT03053063 | Completed - failed in |
| | | | | | second Phase III trial |
| | | | | | (announced Q1 2019) |
| 3 | Cenicriviroc | Allergan | AURORA | NCT03028740 | Recruiting - estimated |
| | | | | | completion Q3 2020 |
| 4 | Elafibranor | GenFit | RESOLVE-IT | NCT02704403 | Completed - failed |
| | | | | | Phase III trial |
| | | | | | (announced Q2 2020) |
| 5 | Obeticholic | Intercept | REGENERATE | NCT02548351 | Completed - positive |
| | acid | | | | interim analysis |
| | | | | | reported Q4 2019; |
| | | | | | received Complete |
| | | | | | Response Letter from |
| | | | | | FDA Q2 2020 |
| | | | | | indicating endpoint |
| | | | | | remains uncertain and |
| | | | | | did not sufficiently |
| | | | | | outweigh the potential |
| | | | | | risks |
| 6 | Obeticholic | Intercept | REVERSE | NCT03439254 | Active - recruitment |
| | acid | | | | completed Q1 2020 |

Given the lack of available treatments, along with the increasing patient population, finding an efficacious therapy remains a growing unmet need.

The short-chain dehydrogenaselreductase (SDR) protein family (H. Jõrnvall et al., Biochemistry 34 (1995), 6003-6013, which is incorporated by reference in its entirety) is a conserved protein family, the members of which show a residue identity level of only 20-30%. However, it has been found that the three-dimensional structure of members of the SDR family are highly similar, determining their functions and affiliation to the SDR family (U. Oppermann et al., Enzymology and Molecular Biology of Carbonyl Metabolism 6, Weiner et al. eds., Plenum Press, New York (1996), p. 403-415, which is incorporated by reference in its entirety).

While initially only two structures of SDR enzymes restricted to bacterial and insect enzymes have been discovered, rapid progress on the knowledge of short chain dehydrogenases/reductases resulted in an increasing number of structures, which could be assigned to the SDR family. Currently, about 1,600 putative members are known, from which up to 100 may be derived from human, such as hydroxysteroid dehydrogenases (HSD), which includes 17β-hydroxysteroid dehydrogenase type 13 (HSD17B13).

Since the SDR enzymes are involved in various metabolic pathways and show different activities, such as oxidoreductases, lyases, or epimerases and show only a low identity of 20-30%, it has been difficult, to assign new members unambiguously to the SDR family and to find modulators therefor.

However, since HSD17B13 and other SDR play a critical role in higher vertebrates, it is desirable to discover further members of the SDR family and establish modulators for known and new SDR enzymes. It was an object of the present disclosure to provide modulators for SDR family members. Still another object of the present disclosure was to provide pharmaceutical agents based on members of the SDR family, including includes short-chain dehydrogenase/reductase family 16C member 3 (SDR16C3), short-chain dehydrogenase/reductase 9 (SDR9), and HSD17B13 (WO2002012544A2).

In a first aspect of the present disclosure, the compounds of Formula (I) are described:

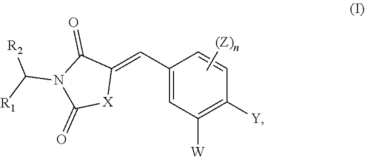

and pharmaceutically acceptable salts, hydrates, solvates, prodrugs, stereoisomers, and tautomers thereof, wherein R₁, R₂, W, X, Y, Z, and n are described herein.

In another aspect of the present disclosure, the compounds of Formula (II) are described:

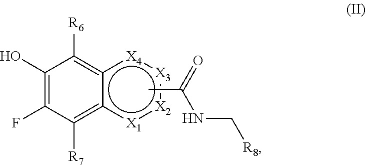

and pharmaceutically acceptable salts, hydrates, solvates, prodrugs, stereoisomers, and tautomers thereof, wherein R₆, R₇, R₈, X₁, X₂, X₃, and X₄ are described herein.

In another aspect of the present disclosure, the compounds of Formula (III) are described:

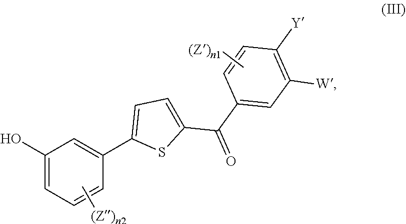

and pharmaceutically acceptable salts, hydrates, solvates, prodrugs, stereoisomers, and tautomers thereof, wherein W′, Y′, Z′, Z″, n1, and n2 are described herein.

The details of the present disclosure are set forth in the accompanying description below. Although methods and materials similar or equivalent to those described herein can be used in the practice or testing of the present disclosure, illustrative methods and materials are now described. Other features, objects, and advantages of the present disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this present disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —COOH, —CH₂CN, —O—(C₁-C₆) alkyl, (C₁-C₆) alkyl, (C₁-C₆) alkoxy, (C₁-C₆) haloalkyl, (C₁-C₆) haloalkoxy, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)NH(C₁-C₆) alkyl, and S(O)N((C₁-C₆) alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆) alkyl, and —S(O)N((C₁-C₆) alkyl)₂. The substituents can themselves be optionally substituted. Furthermore, when containing two fused rings, the aryl groups herein defined may have a saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or polycyclic aromatic radical of 5 to 24 ring atoms, containing one or more ring heteroatoms selected from N, O, S, P, Se, or B, the remaining ring atoms being C. Heteroaryl as herein defined also means a bicyclic heteroaromatic group wherein the heteroatom is selected from N, O, S, P, Se, or B. Heteroaryl as herein defined also means a tricyclic heteroaromatic group containing one or more ring heteroatoms selected from N, O, S, P, Se, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolinyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1λ²-pyrrolo[2,1-b]pyrimidine, dibenzo[b,d] thiophene, pyridin-2-one, furo[3,2-c]pyridinyl, furo[2,3-c]pyridinyl, 1H-pyrido[3,4-b][1,4] thiazinyl, benzoxazolyl, benzisoxazolyl, furo[2,3-b]pyridinyl, benzothiophenyl, 1,5-naphthyridinyl, furo[3,2-b]pyridine, [1,2,4]triazolo[1,5-a]pyridinyl, benzo [1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo [1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, 1H-tetrazolyl, tetrazolyl, 1,2,3-triazolyl, and derivatives thereof. Furthermore, when containing two or more fused rings, the heteroaryl groups defined herein may have one or more saturated or partially unsaturated ring fused with a fully unsaturated aromatic ring, e.g., a 5-membered heteroaromatic ring containing 1 to 3 heteroatoms selected from N, O, S, P, Se, or B, or a 6-membered heteroaromatic ring containing 1 to 3 nitrogens, wherein the saturated or partially unsaturated ring includes 0 to 4 heteroatoms selected from N, O, S, P, Se, or B, and is optionally substituted with one or more oxo. In heteroaryl ring systems containing more than two fused rings, a saturated or partially unsaturated ring may further be fused with a saturated or partially unsaturated ring described herein. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, or benzo[c][1,2]oxaborol-1(3H)-olyl.

“Arylalkyl” refers to an a C₁-C₆ alkyl group, as defined herein, substituted with an aryl ring containing from 3 to 24 ring atoms per ring. For example, arylalkyl groups herein described can have the following formula

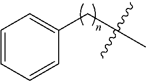

where n is an integer from 1 to 6. Non-limiting examples of suitable aralkyl groups include benzyl, 2-phenethyl and naphthalenylmethyl. The bond to the parent moiety is through the alkyl.

“Halogen” or “halo” refers to fluorine, chlorine, bromine, or iodine.

“Alkyl” refers to a straight or branched chain saturated hydrocarbon containing 1-12 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” refers to a straight or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, t-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above-mentioned monovalent alkyl groups may be an alkylene by abstraction of a second hydrogen atom from the alkyl. As herein defined, alkylene may also be a C₁-C₆alkylene. An alkylene may further be a C₁-C₄alkylene. Typical alkylene groups include, but are not limited to, —CH₂—, —CH(CH)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” means a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, bicyclo[2.2.2]octenyl, decahydronaphthalenyl, octahydro-1H-indenyl, cyclopentenyl, cyclohexenyl, cyclohexa-1,4-dienyl, cyclohexa-1,3-dienyl, 1,2,3,4-tetrahydronaphthalenyl, octahydropentalenyl, 3a,4,5,6,7,7a-hexahydro-1H-indenyl, 1,2,3,3a-tetrahydropentalenyl, bicyclo[3.1.0]hexanyl, bicyclo[2.1.0]pentanyl, spiro[3.3]heptanyl, bicyclo[2.2.1]heptanyl, bicyclo[2.2.1]hept-2-enyl, bicyclo[2.2.2]octanyl, 6-methylbicyclo[3.1.1]heptanyl, 2,6,6-trimethylbicyclo[3.1.1]heptanyl, adamantyl, and derivatives thereof. In the case of polycyclic cycloalkyl, only one of the rings in the cycloalkyl needs to be non-aromatic.

“Heterocyclyl”, “heterocycle” or “heterocycloalkyl” refers to a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms (such as O, N, S, P, Se, or B), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted one or more halogen. Examples of haloalkoxy groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C≡N.

The term “amine” as used herein refers to primary (R—NH₂, R≠H), secondary ((R)₂—NH, R₂≠H) and tertiary ((R)₃—N, R≠H) amines. A substituted amine is intended to mean an amine where at least one of the hydrogen atoms has been replaced by the substituent.

The term “amino” as used herein means a substituent containing at least one nitrogen atom. Specifically, —NH₂, —NH(alkyl) or alkylamino, —N(alkyl)₂ or dialkylamino, amide-, carbamide-, urea, and sulfamide substituents are included in the term “amino”.

The term “solvate” refers to a complex of variable stoichiometry formed by a solute and solvent. Such solvents for the purpose of the present disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I), Formula (II), or Formula (III) may have one or more asymmetric carbon atom and may occur as racemates, racemic mixtures and as individual enantiomers or diastereomers.

The present disclosure also contemplates isotopically-labelled compounds of Formula I (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumerate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” when used in connection with a compound is an amount effective for treating or preventing a condition in a subject as described herein.

The term “carrier” as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug” as used in this disclosure, means a compound which is convertible in vivo by metabolic means (e.g., by hydrolysis) to a disclosed compound.

The present disclosure relates to compounds or pharmaceutically acceptable salts, hydrates, solvates, prodrugs, stereoisomers, or tautomers thereof, capable of inhibiting HSD17B13, which are useful for the treatment of diseases and disorders associated with modulation of HSD17B13. The present disclosure further relates to compounds, or pharmaceutically acceptable salts, hydrates, solvates, prodrugs, stereoisomers, or tautomers thereof, which can be useful for inhibiting HSD17B13.

In some embodiments, the compounds of Formula (I) have the structure of Formula (Ia):

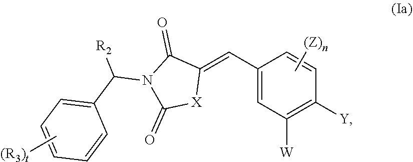

R₃ is selected from —OH, —CF₃, —F, —NHSO₂Me, —S(O)₂—C₁-C₄alkyl, C₁-C₄ alkoxy, C(O)NH₂, C(O)NHR₅, S(O)₂NH₂, S(O)₂NHR₅, and heteroaryl;
   t is an integer selected from 0, 1, 2, and 3; and
   Z is —F.

In some embodiments, R₃ is —OH. In some embodiments, R₃ is —CF₃. In some embodiments, R₃ is —F. In some embodiments, R₃ is —NHSO₂Me. In some embodiments, R₃ is —S(O)₂—C₁-C₄alkyl. In some embodiments, R₃ is C₁-C₄ alkoxy. In some embodiments, R₃ is heteroaryl. In some embodiments, R₃ is C(O)NH₂. In some embodiments, R₃ is C(O)NHR₅. In some embodiments, R₃ is S(O)₂NH₂. In some embodiments, R₃ is S(O)₂NHR₅.

In some embodiments, t is 0. In some embodiments, t is 1. In some embodiments, t is 2. In some embodiments, t is 3.

In some embodiments, the compounds of Formula (I) have the structure of Formula (Ib):

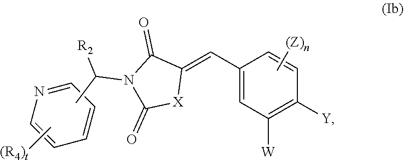

wherein:
R₄ is selected from —OH, —CF₃, —F, —NHSO₂Me, —S(O)₂—C₁-C₄alkyl, C₁-C₄ alkoxy, and heteroaryl;
t is an integer selected from 0, 1, 2, and 3; and
Z is F.

In some embodiments, R₄ is —OH. In some embodiments, R₄ is —CF₃. In some embodiments, R₄ is —F. In some embodiments, R₄ is —NHSO₂Me. In some embodiments, R₄ is —S(O)₂—C₁-C₄alkyl. In some embodiments, R₄ is C₁-C₄ alkoxy. In some embodiments, R₄ is heteroaryl.

In some embodiments, t is 0. In some embodiments, t is 1. In some embodiments, t is 2. In some embodiments, t is 3.

In some embodiments, the compounds of Formula (I) have the structure of Formula (Ic):

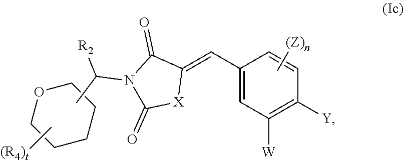

R₄ is selected from —OH, —CF₃, —F, —NHSO₂Me, —S(O)₂—C₁-C₄alkyl, C₁-C₄ alkoxy, and heteroaryl;
t is an integer selected from 0, 1, 2, 3, 4, and 5; and
Z is F.

In some embodiments, R₄ is —OH. In some embodiments, RA is —CF₃. In some embodiments, R₄ is —F. In some embodiments, R₄ is —NHSO₂Me. In some embodiments, R₄ is —S(O)₂—C₁-C₄alkyl. In some embodiments, R₄ is C₁-C₄ alkoxy. In some embodiments, R₄ is heteroaryl.

In some embodiments, t is 0. In some embodiments, t is 1. In some embodiments, t is 2. In some embodiments, t is 3. In some embodiments, t is 4. In some embodiments, t is 5.

In some embodiments, the compounds of Formula (I) have the structure of Formula (Id):

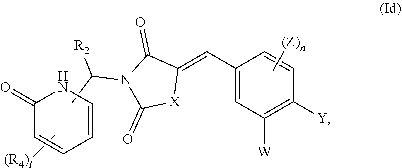

wherein:
R₄ is selected from —OH, —CF₃, —F, —NHSO₂Me, S(O)₂—C₁-C₄alkyl, C₁-C₄ alkoxy, and heteroaryl;
t is an integer selected from 0, 1, 2, and 3; and
Z is F.

In some embodiments, R₄ is —OH. In some embodiments, RA is —CF₃. In some embodiments, R₄ is —F. In some embodiments, R₄ is —NHSO₂Me. In some embodiments, R₄ is —S(O)₂—C₁-C₄alkyl. In some embodiments, R₄ is C₁-C₄ alkoxy. In some embodiments, R₄ is heteroaryl.

In some embodiments, t is 0. In some embodiments, t is 1. In some embodiments, t is 2. In some embodiments, t is 3.

In some embodiments, the compounds of Formula (II) have the structure of Formula (IIa):

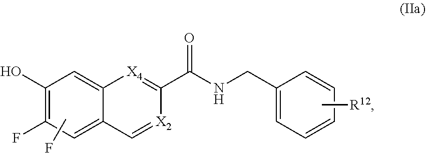

wherein:
R₁₂ is selected from —OH, C₁-C₄ alkoxy, —S(O)₂—C₁-C₄ alkyl, —F, —CN, C₁-C₄ alkyl,

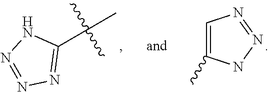

In some embodiments, R₁₂ is —OH. In some embodiments, R₁₂ is —F. In some embodiments, R₁₂ is —S(O)₂—C₁-C₄ alkyl. In some embodiments, R₁₂ is C₁-C₄ alkyl. In some embodiments, R₁₂ is

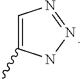

In some embodiments, R₁₂ is —CN. In some embodiments, R₁₂ is

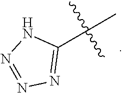 [SMILES: *c1nnn[nH]1]

In some embodiments of the compounds of Formula I, R₁ is independently selected from C₃-C₆ cycloalkyl, tetrahydropyranyl, tetrahydrofuranyl, pyridinyl, piperidinyl, pyrrolidinyl, pyridonyl, phenyl, wherein the phenyl is substituted with one or more R₃; and wherein the cycloalkyl, tetrahydropyranyl, tetrahydrofuranyl, pyridinyl, piperidinyl, pyridonyl, or pyrrolidinyl is optionally substituted with one or more R₄. In some embodiments, R₁ is phenyl. In some embodiments, R₁ is phenyl substituted with one or more R₃. In some embodiments, R₁ is cycloalkyl. In some embodiments, R₁ is cycloalkyl substituted with one or more R₄. In some embodiments, R₁ is tetrahydropyranyl. In some embodiments, R₁ is tetrahydropyranyl substituted with one or more R₁. In some embodiments, R₁ is tetrahydrofuranyl. In some embodiments, R₁ is tetrahydrofuranyl substituted with one or more R₄. In some embodiments, R₁ is pyridinyl. In some embodiments, R₁ is pyridinyl substituted with one or more R₄. In some embodiments, R₁ is piperidinyl. In some embodiments, R₁ is piperidinyl substituted with one or more R₄. In some embodiments, R₁ is pyrrolidinyl. In some embodiments, R₁ is pyrrolidinyl substituted with one or more R₄. In some embodiments, R₁ is pyridone. In some embodiments, R₁ is pyridine substituted with one or more R₄.

In some embodiments, R₁ is selected from the group consisting of

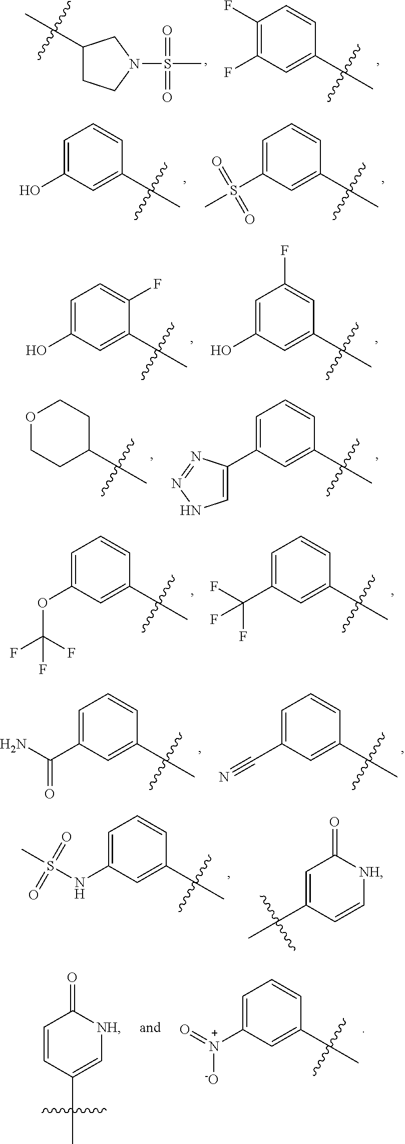

In some embodiments of the compounds of Formula I, R₂ is —H or —CH₃. In some embodiments, R₂ is —H. In some embodiments, R₂ is —CH₃.

In some embodiments of the compounds of Formula I, R₃ is independently selected from halogen, —OH, C₁-C₆ alkoxy, haloalkoxy, —CF₃, —NO₂, —S(O)₂R₅, —NHSO₂C₁-C₄ alkyl, —NHCOC₁-C₄ alkyl, —CF₃, —COOH, —C(O)NH₂, C(O)NH—R₅, S(O)₂NH₂, S(O)₂NHR₅, —CN, —C(O)OR₅, —NH₂, and heteroaryl. In some embodiments, R₃ is halogen. In some embodiments, R₃ is —F. In some embodiments, R₃ is —OH. In some embodiments, R₃ is C₁-C₆ alkoxy. In some embodiments, R₃ is haloalkoxy. In some embodiments, R₃ is —OCF₃. In some embodiments, R₃ is —CF₃. In some embodiments, R₃ is —NO₂. In some embodiments, R₃ is —S(O)₂R₅. In some embodiments, R₃ is —NHSO₂C₁-C₄ alkyl. In some embodiments, R₃ is —NHCOC₁-C₄ alkyl. In some embodiments, R₃ is —CF₃. In some embodiments, R₃ is —COOH. In some embodiments, R₃ is —C(O)NH₂. In some embodiments, R₃ is C(O)NHR₅. In some embodiments, R₃ is S(O)₂NH₂. In some embodiments, R₃ is S(O)₂NHR₅. In some embodiments, R₃ is —CN. In some embodiments, R₃ is —C(O)OR₅. In some embodiments, R₃ is —NH₂. In some embodiments, R₃ is heteroaryl. In some embodiments, R₃ is 5-membered heteroaryl. In some embodiments, R₃ is 5-membered heteroaryl comprising at least one N atom. In some embodiments, R₃ is 5-membered heteroaryl comprising three N atoms.

In some embodiments of the compounds of Formula I, R₄ is oxo, C₁-C₆ alkyl, C₁-C₄ alkoxy, —C(O)R₅, and —S(O)₂R₅. In some embodiments, R₄ is C₁-C₆ alkyl. In some embodiments, R₄ is C₁-C₄ alkoxy. In some embodiments, R₄ is —C(O)R₅. In some embodiments, R₄ is —S(O)₂R₅. In some embodiments, R₄ is —CF₃. In some embodiments, R₄ is oxo.

In some embodiments of the compounds of Formula I, R₅ is independently selected from —H, C₁-C₆ alkyl, C₁-C₆ alkoxy, C₂-C₆ alkenyl, and C₂-C₆ alkynyl. In some embodiments, R₅ is —H. In some embodiments, R₅ is C₁-C₆ alkyl. In some embodiments, R₅ is C₁-C₆ alkoxy. In some embodiments, R₅ is C₂-C₆ alkenyl. In some embodiments, R₅ is C₂-C₆ alkynyl. In some embodiments, R₅ is —CH₃. In some embodiments, R₅ is —CF₃.

In some embodiments of the compounds of Formula I, W is —OH.

In some embodiments of the compounds of Formula I, Y is selected from —H, —OH and —F. In some embodiments, Y is —H. In some embodiments, Y is —OH. In some embodiments, Y is —F.

In some embodiments of Formula I, W and Y, taken together with the atom to which they are each attached, form a 3- to 10-membered heterocycle or heteroaryl, wherein the heterocycle or heteroaryl is optionally substituted with one or more Z. In some embodiments, W and Y, taken together with the atom to which they are each attached, form a 3- to 10-membered heterocycle. In some embodiments, W and Y, taken together with the atom to which they are each attached, form a 3- to 10-membered heteroaryl. In some embodiments, W and Y, taken together with the atom to which they are each attached, form a 5-membered heteroaryl. In some embodiments, W and Y, taken together with the atom to which they are each attached, form a 5-membered heteroaryl comprising at least one N atom. In some embodiments, W and Y, taken together with the atom to which they are each attached, form a 5-membered heteroaryl comprising three N atoms. In some embodiments, W and Y, taken together with the atom to which they are each attached, form 1,2,3-triazole.

In some embodiments of the compounds of Formula I, X is selected from O and S. In some embodiments, X is O. In some embodiments, X is S.

In some embodiments of the compounds of Formula I, Z is independently, at each occurrence, selected from halogen, —OH, —NH₂, —CN, C₁-C₄ alkyl, C₁-C₄ alkoxy, C₂-C₄ alkenyl, and C₂-C₄ alkynyl. In some embodiments, Z is halogen. In some embodiments, Z is —OH. In some embodiments, Z is —NH₂. In some embodiments, Z is —CN. In some embodiments, Z is C₁-C₄ alkyl. In some embodiments, Z is C₁-C₄ alkoxy. In some embodiments, Z is C₂-C₄ alkenyl. In some embodiments, Z is C₂-C₄ alkynyl.

In some embodiments, Z is —F.

In some embodiments of the compounds of Formula I, n is an integer selected from 0, 1, and 2. In some embodiments, n is 0. In some embodiments, n is 1. In some embodiments, n is 2.

In some embodiments of the compounds of Formula II, X₁ is selected from bond, C(R₁₀), and N. In some embodiments, X₁ is bond. In some embodiments, X₁ is C(R₁₀). In some embodiments, X₁ is N.

In some embodiments of the compounds of Formula II, X₂ is selected from C(R₁₀), N, O, and S. In some embodiments, X₂ is C(R₁₀). In some embodiments, X₂ is N. In some embodiments, X₂ is O. In some embodiments, X₂ is S.

In some embodiments of the compounds of Formula II, X₃ is selected from C(R₁₀), N, N(R₁₀), O, and S. In some embodiments, X₃ is C(R₁₀). In some embodiments, X₃ is N. In some embodiments, X₃ is N(R₁₀). In some embodiments, X₃ is O. In some embodiments, X₃ is S.

In some embodiments of the compounds of Formula II, X₄ is selected from C(R₁₀), N, N(R₁₀), O, and S. In some embodiments, X₄ is C(R₁₀). In some embodiments, X₄ is N. In some embodiments, X₄ is N(R₁₀). In some embodiments, X₄ is O. In some embodiments, X₄ is S.

In some embodiments of Formula II, at least one of X₂, X₃, and X₄ are not S or O.

In some embodiments of Formula II, at least one of X₂, X₃, and X₄ is selected from C(R₁₀), N, and N(R₁₀).

In some embodiments of the compounds of Formula II, R₆ is independently selected from —H, halogen, —NR₁₀R₁₁, —C(O)R₁₀, —C(O)NR₁₀R₁₁, —CN, C₁-C₆ alkyl, C₁-C₆ alkoxy, C₂-C₆ alkenyl, C₂-C₆ alkynyl, C₃-C₁₀ cycloalkyl, C₅-C₈ cycloalkenyl, C₅-C₈ spirocycloalkyl, spiroheterocyclyl, heterocyclyl, aryl, and heteroaryl, wherein the alkyl, alkoxy, alkenyl, alkynyl, cycloalkyl, cycloalkenyl, spirocycloalkyl, spiroheterocyclyl, heterocyclyl, aryl, or heteroaryl is optionally substituted with one or more R₉. In some embodiments, R₆ is —H, halogen. In some embodiments, R₇ is halogen. In some embodiments, R₆ is —NR₁₀R₁₁. In some embodiments, R₆ is —C(O)R₁₀. In some embodiments, R₆ is —C(O)NR₁₀R₁₁. In some embodiments, R₆ is —CN. In some embodiments, R₆ is C₁-C₆ alkyl. In some embodiments, R₆ is C₁-C₆ alkoxy. In some embodiments, R₆ is C₂-C₆ alkenyl. In some embodiments, R₆ is C₂-C₆ alkynyl. In some embodiments, R₆ is C₃-C₁₀ cycloalkyl. In some embodiments, R₆ is C₅-C₈ cycloalkenyl. In some embodiments, R₆ is C₅-C₈ spirocycloalkyl. In some embodiments, R₆ is spiroheterocyclyl. In some embodiments, R₆ is heterocyclyl. In some embodiments, R₆ is aryl. In some embodiments, R₆ is heteroaryl.

In some embodiments of the compounds of Formula II, R₇ is independently selected from —H, halogen, —NR₁₀R₁₁, —C(O)R₁₀, —C(O)NR₁₀R₁₁, —CN, C₁-C₆ alkyl, C₁-C₆, alkoxy, C₂-C₆ alkenyl, C₂-C₆ alkynyl, C₃-C₁₀ cycloalkyl, C₅-C₈cycloalkenyl, C₅-C₈ spirocycloalkyl, spiroheterocyclyl, heterocyclyl, aryl, and heteroaryl, wherein the alkyl, alkoxy, alkenyl, alkynyl, cycloalkyl, cycloalkenyl, spirocycloalkyl, spiroheterocyclyl, heterocyclyl, aryl, or heteroaryl is optionally substituted with one or more R₉. In some embodiments, R₇ is —H, halogen. In some embodiments, R₇ is halogen. In some embodiments, R₇ is —NR₁₀R₁₁. In some embodiments, R₇ is —C(O)R₁₀. In some embodiments, R₁₀ is —C(O)NR₁₀R₁₁. In some embodiments, R₇ is —CN. In some embodiments, R₇ is C₁-C₆ alkyl. In some embodiments, R₇ is C₁-C₆ alkoxy. In some embodiments, R₇ is C₂-C₆ alkenyl. In some embodiments, R₇ is C₂-C₆ alkynyl. In some embodiments, R₇ is C₃-C₁₀ cycloalkyl. In some embodiments, R₇ is C₅-C₈ cycloalkenyl. In some embodiments, R₇ is C₅-C₈ spirocycloalkyl. In some embodiments, R₇ is spiroheterocyclyl. In some embodiments, R₇ is heterocyclyl. In some embodiments, R₇ is aryl. In some embodiments, R₇ is heteroaryl.

In some embodiments of the compounds of Formula II, R₈ is selected from phenyl and heteroaryl, wherein the phenyl or heteroaryl is optionally substituted with one or more —OH, C₁-C₄ alkoxy, —S(O)₂—C₁-C₃ alkyl, —COOH, —CN, —CONH₂, or heteroaryl containing 1-4 heteroatoms selected from N, O, and S. In some embodiments, R₈ is phenyl. In some embodiments, R₈ is heteroaryl. In some embodiments, R₈ is phenyl substituted with one or more —OH. In some embodiments, R₈ is phenyl substituted with one —OH. In some embodiments, R₈ is phenyl substituted with one or more C₁-C₄ alkoxy. In some embodiments, R₈ is phenyl substituted with one C₁-C₄ alkoxy. In some embodiments, R₈ is phenyl substituted with one or more methoxy. In some embodiments, R₈ is phenyl substituted with one methoxy. In some embodiments, R₈ is phenyl substituted with one or more —CN. In some embodiments, R₈ is phenyl substituted with one —CN. In some embodiments, R₈ is phenyl substituted with one or more heteroaryl. In some embodiments, R₈ is phenyl substituted with heteroaryl. In some embodiments, R₈ is phenyl substituted with one or more 5-membered heteroaryl. In some embodiments, R₈ is phenyl substituted with 5-membered heteroaryl. In some embodiments, R₈ is phenyl substituted with one or more 5-membered heteroaryl comprising four N atoms. In some embodiments, R₈ is phenyl substituted with 5-membered heteroaryl comprising four N atoms.

In some embodiments of the compounds of Formula II, R₈ is selected from

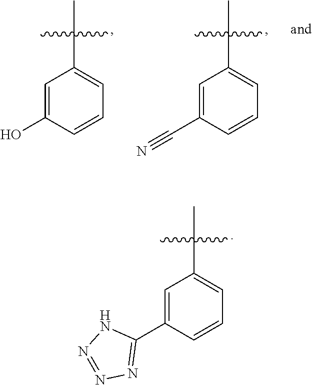 [SMILES: *c1cccc(O)c1 ; *c1cccc(C#N)c1 ; *c1cccc(-c2nnn[nH]2)c1]

In some embodiments of the compounds of Formula II, R₉ is selected from —H, halogen, C₁-C₆ alkyl, C₁-C₆ alkoxy, C₂-C₆ alkenyl, C₂-C₆ alkynyl, C₃-C₈ cycloalkyl, heterocyclyl, aryl, and heteroaryl. In some embodiments, R₉ is —H. In some embodiments, R₉ is halogen. In some embodiments, R₉ is C₁-C₆ alkyl. In some embodiments, R₉ is C₁-C₆ alkoxy. In some embodiments, R₉ is C₂-C₆ alkenyl. In some embodiments, R₉ is C₂-C₆ alkynyl. In some embodiments, R₉ is C₃-C₈ cycloalkyl. In some embodiments, R₉ is heterocyclyl. In some embodiments, R₉ is aryl. In some embodiments, R₉ is heteroaryl.

In some embodiments of the compounds of Formula II, R₁₀ is selected from —H, halogen, C₁-C₆ alkyl, C₁-C₆ alkoxy, C₂-C₆ alkenyl, C₂-C₆ alkynyl, C₃-C₈ cycloalkyl, heterocyclyl, aryl, and heteroaryl. In some embodiments, R₁₀ is —H. In some embodiments, R₁₀ is halogen. In some embodiments, R₁₀ is C₁-C₆ alkyl. In some embodiments, R₁₀ is C₁-C₆ alkoxy. In some embodiments, R₁₀ is C₂-C₆ alkenyl. In some embodiments, R₁₀ is C₂-C₆ alkynyl. In some embodiments, R₁₀ is C₃-C₈ cycloalkyl. In some embodiments, R₁₀ is heterocyclyl. In some embodiments, R₁₀ is aryl. In some embodiments, R₁₀ is heteroaryl.

In some embodiments of the compounds of Formula II, R₁₁ is selected from —H, halogen, C₁-C₆ alkyl, C₁-C₆ alkoxy, C₂-C₆ alkenyl, C₂-C₆ alkynyl, C₃-C₈ cycloalkyl, heterocyclyl, aryl, and heteroaryl. In some embodiments, R₁₁ is —H. In some embodiments, R₁₁ is halogen. In some embodiments, R₁₁ is C₁-C₆ alkyl. In some embodiments, R₁₁ is C₁-C₆ alkoxy. In some embodiments, R₁₁ is C₂-C₆ alkenyl. In some embodiments, R₁₁ is C₂-C₆ alkynyl. In some embodiments, R₁₁ is C₅-C₈ cycloalkyl. In some embodiments, R₁₁ is heterocyclyl. In some embodiments, R₁₁ is aryl. In some embodiments, R₁₁ is heteroaryl.

In some embodiments of the compounds of Formula III, W′ is selected from —H, —OH, and —F. In some embodiments, W′ is —H. In some embodiments, W′ is —OH. In some embodiments, W′ is —F.

In some embodiments of the compounds of Formula III, Y′ is selected from —OH and —F. In some embodiments, YW′ is —OH. In some embodiments, Y′ is —F.

In some embodiments of the compounds of Formula III, each Z′ is independently, at each occurrence, selected from halogen, —OH, —NH₂, —CN, C₁-C₄ alkyl, C₁-C₄ alkoxy, C₂-C₄ alkenyl, and C₂-C₄ alkynyl. In some embodiments, Z′ is halogen. In some embodiments, Z′ is —OH. In some embodiments, Z′ is —NH₂. In some embodiments, Z′ is —CN. In some embodiments, Z′ is C₁-C₄ alkyl. In some embodiments, Z′ is C₁-C₄ alkoxy. In some embodiments, Z′ is C₂-C₄ alkenyl. In some embodiments, Z′ is C₂-C₄ alkynyl. In some embodiments, Z′ is —F. In some embodiments, Z′ is —Cl. In some embodiments, Z′ is —Br. In some embodiments, Z′ is —I.

In some embodiments of the compounds of Formula III, each Z″ is independently, at each occurrence, selected from halogen, —OH, —NH₂, —CN, C₁-C₄ alkyl, C₁-C₄ alkoxy, C₂-C₄ alkenyl, and C₂-C₄ alkynyl. In some embodiments, Z″ is halogen. In some embodiments, Z″ is —OH. In some embodiments, Z″ is —NH₂. In some embodiments of the compounds, Z″ is —CN. In some embodiments, Z′ is C₁-C₄ alkyl. In some embodiments, Z″ is C₁-C₄ alkoxy. In some embodiments, Z″ is C₂-C₄ alkenyl. In some embodiments, Z″ is C₂-C₄ alkynyl. In some embodiments, Z″ is —F. In some embodiments, Z″ is —Cl. In some embodiments, Z″ is —Br. In some embodiments, Z″ is —I.

In some embodiments of the compounds of Formula III, n1 is an integer selected from 0, 1, 2, and 3. In some embodiments, n1 is 0. In some embodiments, n1 is 1. In some embodiments, n1 is 2. In some embodiments, n1 is 3.

In some embodiments of the compounds of Formula III, n2 is an integer selected from 0, 1, 2, 3, and 4. In some embodiments, n2 is 0. In some embodiments, n2 is 1. In some embodiments, n2 is 2. In some embodiments, n2 is 3. In some embodiments, n2 is 4.

In some embodiments of the compounds of Formula I, R₁ is phenyl, R₂ is H, R₃ is —OH, W is —OH, X is S, Y is —F, Z is —F, and n is 2.

In some embodiments of the compounds of Formula I, R₁ is phenyl, R₂ is H, R₃ is —S(O)₂R₅, R₅ is C₁-C₆ alkyl, W is —OH, X is S. Y is —F, Z is —F, and n is 2.

In some embodiments of the compounds of Formula I, R₁ is phenyl, R₂ is H, R₃ is —OH, W is —OH, X is S, Y is —F, Z is —F, and n is 1.

In some embodiments of the compounds of Formula I, R₁ is phenyl, R₂ is H, R₃ is —S(O)₂R₅, R₅ is C₁-C₆ alkyl, W is —OH, X is S. Y is —F, and n is 0.

In some embodiments of the compounds of Formula I, R₁ is tetrahydropyranyl, R₂ is H, W is —OH, X is O, Y is —F, and n is 0.

In some embodiments of the compounds of Formula I, R₁ is phenyl, R₂ is H, R₃ is heteroaryl, W is —OH, X is S, Y is —F, and n is 0.

In some embodiments of the compounds of Formula I, R₁ is phenyl, R₂ is H, R₃ is —CF₃, W is —OH, X is S, Y is —F, and n is 0.

In some embodiments of the compounds of Formula I, R₁ is phenyl, R₂ is H, R₃ is —C(O)NH₂, W is —OH, X is S, Y is —F, and n is 0.

In some embodiments of the compounds of Formula I, R₁ is phenyl, R₂ is H, R₃ is —CN, W is —OH, X is S, Y is —F, and n is 0.

In some embodiments of the compounds of Formula I, R₁ is pyridine, R₂ is H, R₄ is C₁-C₄ alkoxy, W is —OH, X is S, Y is —F, Z is —F, and n is 2.

In some embodiments of the compounds of Formula I, R₁ is phenyl, R₂ is H, R₃ is C₁-C₄ alkoxy, W is —OH, X is S, Y is —F, Z is —F, and n is 2.

In some embodiments of the compounds of Formula I, R₁ is phenyl, R₂ is H, R₃ is S(O)₂NH₂, W is —OH, X is S, Y is —F, Z is —F, and n is 2.

In some embodiments of the compounds of Formula I, R₁ is phenyl, R₂ is H, R₃ is —OH, X is S. and W and Y, taken together with the atom to which they are each attached, form a 5-membered heteroaryl

In some embodiments of the compounds of Formula II, R is H, R₇ is H. R is phenyl, X₁ is C(R₁₀), X₂ is C(R₁₀), X₃ is C(R₁₀), X₄ is N, and each R₁₀ is H.

In some embodiments of the compounds of Formula II, R₆ is F, R₇ is H, R₈ is phenyl substituted with C₁-C₄ alkoxy, X₁ is C(R₁₀), X₂ is C(R₁₀), X₃ is C(R₁₀), X₄ is N, and each R₁₀ is H.

In some embodiments of the compounds of Formula III, W′ is —OH. Y is —F, Z″ is —F, n1 is 0, and n2 is 2.

In some embodiments of the compounds of Formula III, W′ is —OH, Y is —F, Z′ is F, Z″ is —F, n1 is 2, and n2 is 2.

In some embodiments of the compounds of Formula III, W′ is —OH, Y is —F, Z′ is F, Z″ is —F, n1 is 1, and n2 is 2.

Non-limiting illustrative compounds of the present disclosure include:
(5Z)-3-[(1-methanesulfonylpyrrolidin-3-yl)methyl]-5-[(2,4,6-trifluoro-3-hydroxyphenyl)methylidene]-1,3-thiazolidine-2,4-dione;
(5Z)-3-[(3,4-difluorophenyl)methyl]-5-[(4-fluoro-3-hydroxyphenyl)methylidene]-1,3-thiazolidine-2,4-dione;
(5Z)-3-[(3-hydroxyphenyl)methyl]-5-[(2,4,5-trifluoro-3-hydroxyphenyl)methylidene]-1,3-thiazolidine-2,4-dione;
(5Z)-3-[(3-hydroxyphenyl)methyl]-5-[(2,4,6-trifluoro-3-hydroxyphenyl)methylidene]-1,3-thiazolidine-2,4-dione;
(5Z)-3-[(3-methanesulfonylphenyl)methyl]-5-[(2,4,6-trifluoro-3-hydroxyphenyl)methylidene]-1,3-thiazolidine-2,4-dione;
(5Z)-5-[(2,4-difluoro-3-hydroxyphenyl)methylidene]-3-[(3-hydroxyphenyl)methyl]-1,3-thiazolidine-2,4-dione;
(5Z)-5-[(3,4-difluoro-5-hydroxyphenyl)methylidene]-3-[(3-hydroxyphenyl)methyl]-1,3-thiazolidine-2,4-dione;
(5Z)-5-[(4-fluoro-3-hydroxyphenyl)methylidene]-3-[(2-fluoro-5-hydroxyphenyl)methyl]-1,3-thiazolidine-2,4-dione;
(5Z)-5-[(4-fluoro-3-hydroxyphenyl)methylidene]-3-[(3-fluoro-5-hydroxyphenyl)methyl]-1,3-thiazolidine-2,4-dione;
(5Z)-5-[(4-fluoro-3-hydroxyphenyl)methylidene]-3-[(3-hydroxyphenyl)methyl]-1,3-oxazolidine-2,4-dione;
(5Z)-5-[(4-fluoro-3-hydroxyphenyl)methylidene]-3-[(3-hydroxyphenyl)methyl]-1,3-thiazolidine-2,4-dione;
(5Z)-5-[(4-fluoro-3-hydroxyphenyl)methylidene]-3-[(3-methanesulfonylphenyl)methyl]-1,3-thiazolidine-2,4-dione;
(5Z)-5-[(4-fluoro-3-hydroxyphenyl)methylidene]-3-[(oxan-4-yl)methyl]-1,3-oxazolidine-2,4-dione;
(Z)-5-[(4-fluoro-3-hydroxyphenyl)methylidene]-3-[(oxan-4-yl)methyl]-1,3-thiazolidine-2,4-dione;
(5Z)-5-[(4-fluoro-3-hydroxyphenyl)methylidene]-3-([3-(1H-1,2,3-triazol-4-yl)phenyl]methyl)-1,3-thiazolidine-2,4-dione;
(5Z)-5-[(4-fluoro-3-hydroxyphenyl)methylidene]-3-{[3-(trifluoromethoxy)phenyl]methyl}-1,3-thiazolidine-2,4-dione;
(5Z)-5-[(4-fluoro-3-hydroxyphenyl)methylidene]-3-{[3-(trifluoromethyl)phenyl]methyl}-1,3-thiazolidine-2,4-dione;
3-{[(5Z)-5-[(4-fluoro-3-hydroxyphenyl)methylidene]-2,4-dioxo-1,3-thiazolidin-3-yl]methyl}benzamide;
3-{[(5Z)-5-[(4-fluoro-3-hydroxyphenyl)methylidene]-2,4-dioxo-1,3-thiazolidin-3-yl]methyl}benzonitrile;
N-(3-([(5Z)-5-[(4-fluoro-3-hydroxyphenyl)methylidene]-2,4-dioxo-1,3-thiazolidin-3-yl]methyl)phenyl)methanesulfonamide;
(Z)-5-(4-fluoro-3-hydroxybenzylidene)-3-(3-nitrobenzyl)thiazolidine-2,4-dione;
(5Z)-3-[(2-methoxypyridin-4-yl)methyl]-5-[(2,4,6-trifluoro-3-hydroxyphenyl)methylidene]-1,3-thiazolidine-2,4-dione;
(5Z)-3-[(6-methoxypyridin-3-yl)methyl]-5-[(2,4,6-trifluoro-3-hydroxyphenyl)methylidene]-1,3-thiazolidine-2,4-dione;
6-fluoro-7-hydroxy-N-[(3-hydroxyphenyl)methyl]quinoline-2-carboxamide;
6-fluoro-7-hydroxy-N-{[3-(1H-1,2,3,4-tetrazol-5-yl)phenyl]methyl}quinoline-2-carboxamide;
7-fluoro-6-hydroxy-N-[(3-hydroxyphenyl)methyl]isoquinoline-3-carboxamide;
N-[(3-cyanophenyl)methyl]-6-fluoro-7-hydroxyquinoline-2-carboxamide;
2,6-difluoro-3-[5-(4-fluoro-3-hydroxybenzoyl)thiophen-2-yl]phenol;
3-[5-(2,4-difluoro-3-hydroxyphenyl)thiophene-2-carbonyl]-2,4,6-trifluorophenol;
3-[5-(3,4-difluoro-5-hydroxybenzoyl)thiophen-2-yl]-2,6-difluorophenol;
3-[5-(2,4-difluoro-3-hydroxyphenyl)thiophene-2-carbonyl]-2,5,6-trifluorophenol;
3-[5-(2,4-difluoro-3-hydroxybenzoyl)thiophen-2-yl]-2,6-difluorophenol;
3-{[(5Z)-5-[(4-fluoro-3-hydroxyphenyl)methylidene]-2,4-dioxo-1,3-thiazolidin-3-yl]methyl}benzene-1-sulfonamide;
3-{[(5Z)-2,4-dioxo-5-[(2,4,6-trifluoro-3-hydroxyphenyl)methylidene]-1,3-thiazolidin-3-yl]methyl}benzene-1-sulfonamide;
(5Z)-3-[(6-oxo-1,6-dihydropyridin-3-yl)methyl]-5-[(2,4,6-trifluoro-3-hydroxyphenyl)methylidene]-1,3-thiazolidine-2,4-dione;
(5Z)-3-[(2-oxo-1,2-dihydropyridin-4-yl)methyl]-5-[(2,4,6-trifluoro-3 hydroxyphenyl)methylidene]-1,3-thiazolidine-2,4-dione;
(5Z)-5-[(2,6-difluoro-3-hydroxyphenyl)methylidene]-3-[(3-hydroxyphenyl)methyl]-1,3-thiazolidine-2,4-dione;
(5Z)-5-[(1H-1,2,3-benzotriazol-6-yl)methylidene]-3-[(3-hydroxyphenyl)methyl]-1,3-thiazolidine-2,4-dione; and
6,8-difluoro-7-hydroxy-N-[(3-methoxyphenyl)methyl]quinoline-2-carboxamide.

It should be understood that all isomeric forms are included within the present disclosure, including mixtures thereof. If the compound contains a double bond, the substituent may be in the E or Z configuration. If the compound contains a disubstituted cycloalkyl, the cycloalkyl substituent may have a cis- or trans configuration. All tautomeric forms are also intended to be included.

Compounds of the present disclosure, and pharmaceutically acceptable salts, hydrates, solvates, stereoisomers and prodrugs thereof may exist in their tautomeric form (for example, as an amide or imino ether). All such tautomeric forms are contemplated herein as part of the present disclosure.

The compounds of the present disclosure may contain asymmetric or chiral centers, and, therefore, exist in different stereoisomeric forms. It is intended that all stereoisomeric forms of the compounds of the present disclosure as well as mixtures thereof, including racemic mixtures, form part of the present disclosure. In addition, the present disclosure embraces all geometric and positional isomers. For example, if a compound of the present disclosure incorporates a double bond or a fused ring, both the cis- and trans-forms, as well as mixtures, are embraced within the scope of the present disclosure. each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the present disclosure may be atropisomers (e.g., substituted biaryls) and are considered as part of this present disclosure. Enantiomers can also be separated by use of a chiral HPLC column.

It is also possible that the compounds of the present disclosure may exist in different tautomeric forms, and all such forms are embraced within the scope of the present disclosure. Also, for example, all keto-enol and imine-enamine forms of the compounds are included in the present disclosure.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this present disclosure, as are positional isomers (such as, for example, 4-pyridyl and 3-pyridyl). (For example, if a compound of Formula (I), Formula (II), or Formula (III) incorporates a double bond or a fused ring, both the cis- and trans-forms, as well as mixtures, are embraced within the scope of the present disclosure. Also, for example, all keto-enol and imine-enamine forms of the compounds are included in the present disclosure). Individual stereoisomers of the compounds of the present disclosure may, for example, be substantially free of other isomers, or may be admixed, for example, as racemates or with all other, or other selected, stereoisomers. The chiral centers of the present disclosure can have the S or R configuration as defined by the TUPAC 1974 Recommendations. The use of the terms “salt”, “solvate”, “ester,” “prodrug” and the like, is intended to equally apply to the salt, solvate, ester and prodrug of enantiomers, stereoisomers, rotamers, tautomers, positional isomers, racemates or prodrugs of the inventive compounds.

The compounds of Formula I may form salts which are also within the scope of this present disclosure. Reference to a compound of the Formula herein is understood to include reference to salts thereof, unless otherwise indicated.

The present disclosure relates to compounds which are modulators of SDR enzymes. In one embodiment, the compounds of the present disclosure are inhibitors of SDR enzymes. In another embodiment, the SDR enzyme is short-chain dehydrogenase/reductase family 16C member 3 (SDR16C3). In another embodiment, the SDR enzyme is short-chain dehydrogenase/reductase 9 (SDR9). In another embodiment, the SDR enzyme is 17@f-hydroxysteroid dehydrogenase type 13 (HSD17B13).

The present disclosure is directed to compounds as described herein and pharmaceutically acceptable salts, hydrates, solvates, prodrugs, stereoisomers, or tautomers thereof, and pharmaceutical compositions comprising one or more compounds as described herein, or pharmaceutically acceptable salts, hydrates, solvates, prodrugs, stereoisomers, or tautomers thereof.

### Method of Synthesizing the Compounds

The compounds of the present disclosure may be made by a variety of methods, including standard chemistry. Suitable synthetic routes are depicted in the Schemes given below.

The compounds of Formula (I), Formula (II), or Formula (III) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (I), Formula (II), or Formula (III). Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-lnterscience, 1994).

The compounds described herein may be made from commercially available starting materials or synthesized using known organic, inorganic, and/or enzymatic processes.

### Preparation of Compounds

The compounds of the present disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Preferred methods include but are not limited to those methods described below. Compounds of the present disclosure can be synthesized by following the steps outlined in General Procedures I-V which comprise different sequences of assembling intermediates or compounds. Starting materials are either commercially available or made by known procedures in the reported literature or as illustrated below.

General Procedure I:

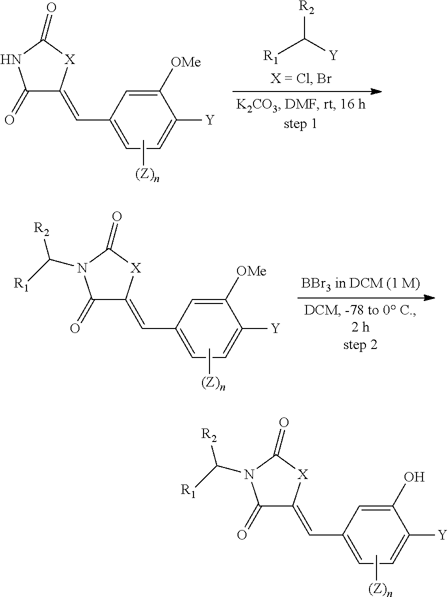

General Procedure II:

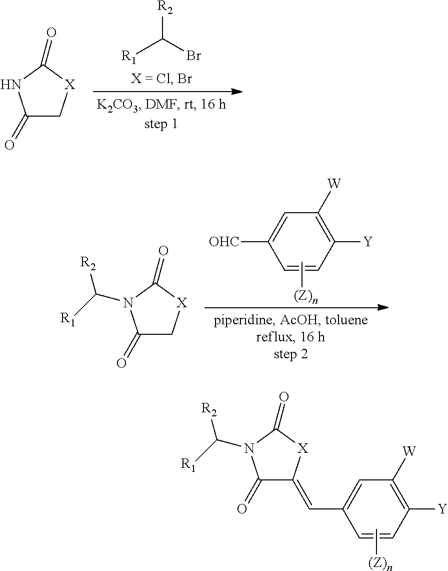

General Procedure III:

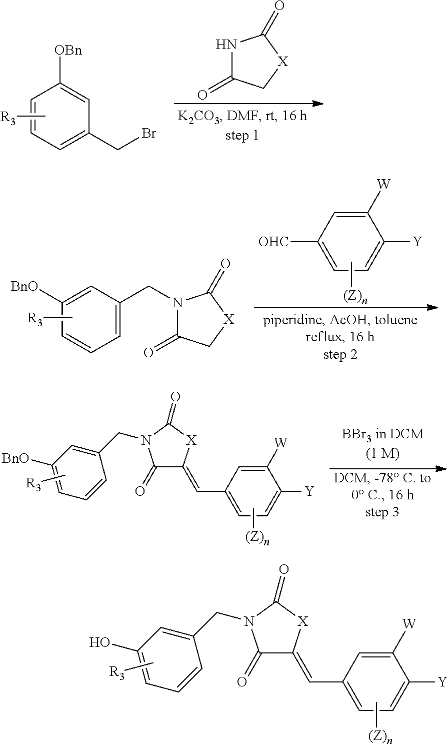

General Procedure IV:

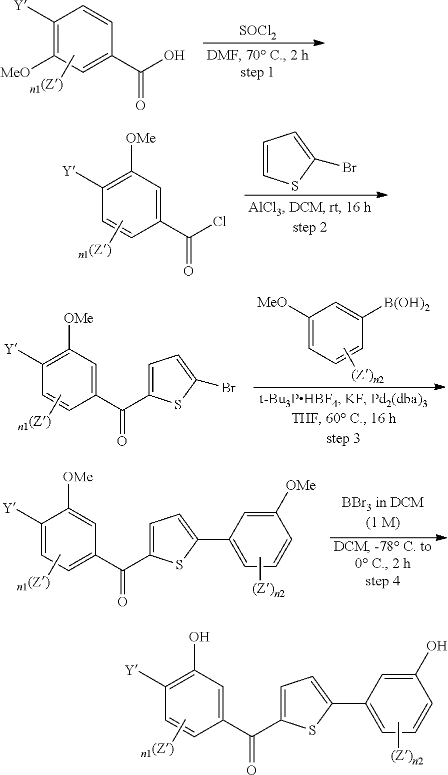

General Procedure V:

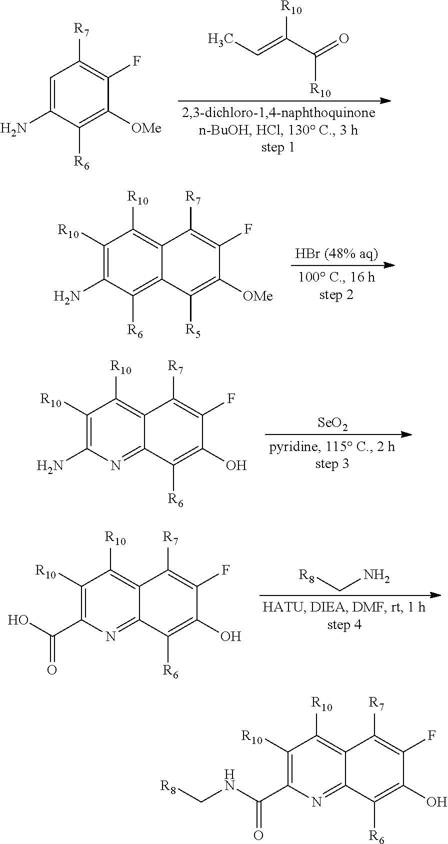

### Methods of Using the Disclosed Compounds

Another aspect of the present disclosure relates to a method of treating a condition associated with modulation of HSD17B13. The method comprises administering to a patient in need of a treatment for conditions associated with modulation of HSD17B13 an effective amount the compositions and compounds of Formula (I), Formula (II), or Formula (III).

In another aspect, the present disclosure is directed to a method of inhibiting HSD17B13. The method involves administering to a patient in need thereof an effective amount of a compound of Formula (I), Formula (II), or Formula (III).

Another aspect of the present disclosure relates to a method of treating, preventing, inhibiting or eliminating a condition in a patient associated with the inhibition of HSD17B13, the method comprising administering to a patient in need thereof an effective amount of a compound of Formula (I), Formula (II), or Formula (III). In one embodiment, the disease or condition may be, but not limited to, primary sclerosing cholangitis, chronic liver disease, nonalcoholic fatty liver disease (NAFLD), nonalcoholic steatohepatitis (NASH), hepatitis C, alcoholic liver disease, liver damage due to progressive fibrosis, and liver fibrosis. In one embodiment, the condition is nonalcoholic steatohepatitis (NASH).

The present disclosure also relates to the use of an inhibitor of HSD17B13 for the preparation of a medicament used in the treatment, prevention, inhibition or elimination of a disease or condition mediated by HSD17B13, wherein the medicament comprises a compound of Formula (I), Formula (II), or Formula (III).

In another aspect, the present disclosure relates to a method for the manufacture of a medicament for treating, preventing, inhibiting, or eliminating a disease or condition mediated by HSD17B13, wherein the medicament comprises a compound of Formula (I), Formula (II), or Formula (In).

Another aspect of the present disclosure relates to a compound of Formula (I), Formula (H), or Formula (III) for use in the manufacture of a medicament for treating a disease associated with inhibiting HSD17B13.

In another aspect, the present disclosure relates to the use of a compound of Formula (I), Formula (I), or Formula (I) in the treatment of a disease associated with inhibiting HSD17B13.

Another aspect of the present disclosure relates to a method of treating primary sclerosing cholangitis. The method comprises administering to a patient in need thereof an effective amount of a compound of Formula (I), Formula (II), or Formula (III).

Another aspect of the present disclosure relates to a method of preventing primary sclerosing cholangitis. The method comprises administering to a patient in need thereof an effective amount of a compound of Formula (I), Formula (II), or Formula (III).

In another aspect of the present disclosure, the method relates to treating chronic liver disease. The method comprises administering to a patient in need thereof an effective amount of a compound of Formula (I), Formula (II), or Formula (III).

In another aspect of the present disclosure, the method relates to preventing chronic liver disease. The method comprises administering to a patient in need thereof an effective amount of a compound of Formula (I), Formula (II), or Formula (III).

In another aspect of the present disclosure, the method relates to treating nonalcoholic fatty liver disease (NAFLD). The method comprises administering to a patient in need thereof an effective amount of a compound of Formula (I), Formula (II), or Formula (III).

In another aspect of the present disclosure, the method relates to preventing nonalcoholic fatty liver disease (NAFLD). The method comprises administering to a patient in need thereof an effective amount of a compound of Formula (I), Formula (II), or Formula (III).

In another aspect of the present disclosure, the method relates to treating nonalcoholic steatohepatitis (NASH). The method comprises administering to a patient in need thereof an effective amount of a compound of Formula (I), Formula (II), or Formula (III).

In another aspect of the present disclosure, the method relates to preventing nonalcoholic steatohepatitis (NASH). The method comprises administering to a patient in need thereof an effective amount of a compound of Formula (I), Formula (II), or Formula (III).

In another aspect of the present disclosure, the method relates to treating hepatitis C. The method comprises administering to a patient in need thereof an effective amount of a compound of Formula (I), Formula (II), or Formula (III).

In another aspect of the present disclosure, the method relates to preventing hepatitis C. The method comprises administering to a patient in need thereof an effective amount of a compound of Formula (I), Formula (II), or Formula (III).

In another aspect of the present disclosure, the method relates to treating alcoholic liver disease. The method comprises administering to a patient in need thereof an effective amount of a compound of Formula (I), Formula (II), or Formula (III).

In another aspect of the present disclosure, the method relates to preventing alcoholic liver disease. The method comprises administering to a patient in need thereof an effective amount of a compound of Formula (I), Formula (II), or Formula (III).

In another aspect of the present disclosure, the method relates to treating liver damage due to progressive fibrosis. The method comprises administering to a patient in need thereof an effective amount of a compound of Formula (I), Formula (II), or Formula (III).

In another aspect of the present disclosure, the method relates to preventing liver damage due to progressive fibrosis. The method comprises administering to a patient in need thereof an effective amount of a compound of Formula (I), Formula (II), or Formula (III).

In another aspect of the present disclosure, the method relates to treating liver fibrosis. The method comprises administering to a patient in need thereof an effective amount of a compound of Formula (I), Formula (II), or Formula (III).

In another aspect of the present disclosure, the method relates to preventing liver fibrosis. The method comprises administering to a patient in need thereof an effective amount of a compound of Formula (I), Formula (II), or Formula (III).

In one embodiment, the present disclosure relates to the use of an inhibitor of HSD17B13 for the preparation of a medicament used in treatment, prevention, inhibition or elimination of a condition selected from primary sclerosing cholangitis, chronic liver disease, nonalcoholic fatty liver disease (NAFLD), nonalcoholic steatohepatitis (NASH), hepatitis C, alcoholic liver disease, liver damage due to progressive fibrosis, and liver fibrosis.

In one embodiment, the subject does not experience an increase in pruritis.

In one embodiment, the subject does not experience a statistically significant increase in plasma triglycerides, LDL, or cholesterol.

In one embodiment, the subject experiences hepatic histological improvement in NASH by greater than or equal to 2 points with at least 1-point reduction in either lobular inflammation or hepatocellular ballooning and no concurrent worsening of fibrosis.

In one embodiment, the subject experiences a decrease in hepatic fat.

In one embodiment, the subject experiences an improvement in serum ALT, AST, and/or GGT.

In one embodiment, the subject experiences a reduced in markers of bile acid synthesis.

In one embodiment, the subject was not responsive to a prior treatment with selonsertib, cenicriviroc, elafibrinor, obeticholic acid, or any combination thereof.

In one embodiment, the subject is a mammal.

In one embodiment, the mammal is a human.

In one embodiment, are provided methods of treating a condition associated with modulation of HSD17B13, including primary sclerosing cholangitis, chronic liver disease, nonalcoholic fatty liver disease (NAFLD), nonalcoholic steatohepatitis (NASH), hepatitis C, alcoholic liver disease, liver damage due to progressive fibrosis, and liver fibrosis, comprising administering to a patient suffering from at least one of said diseases or disorder a compound of Formula (I), Formula (II), or Formula (III).

In one embodiment, the subject exhibits at least one of; (a) at least 1 stage improvement in fibrosis without worsening of NASH; (b) reduction of resolution of NASH without worsening of NASH; (c) event-free survival (EFS); (d) cirrhosis-free survival; (e) reduction in complications of ascites; (f) transplant-free survival; (g) variceal hemorrhage-free survival, (h) hepatic encephalopathy-free survival; (i) improvement in Model For End-Stage Liver Disease (MELD) score; and (j) survival.

In one embodiment, the subject exhibits at least one of; (a) improvement of fibrosis by at least 1 stage and/or resolution of NASH, without worsening of either; (b) no worsening of fibrosis and no worsening of NASH; (c) improvement in each histological feature of NASH by at least 1 point; (d) improvement of fibrosis by at least 2 stages; (e) improvement in NASH by at least 2 points with no worsening of fibrosis; (f) NASH resolution with no worsening of fibrosis; (g) improvement of fibrosis and resolution of NASH as a composite endpoint and as defined by both endpoints being met in the same subject; (h) resolution of fibrosis; or (i) no histological progression to cirrhosis.

In one embodiment, the subject exhibits at least one stage, at least two stages, at least three stages, or more of liver fibrosis improvement.

In one embodiment, the improvement is determined histologically.

In one embodiment, the stage of liver fibrosis improvement comprises no worsening of NASH.

In one embodiment, the stage of liver fibrosis improvement comprises an improvement of NASH.

In one embodiment, the subject exhibits NASH resolution.

In one embodiment, the subject further exhibits no worsening of liver fibrosis.

In one embodiment, the stage of liver fibrosis improvement is statistically significant.

In one embodiment, the improvement is compared to a control.

In one embodiment, the improvement occurs within about 4 weeks, about 8 weeks, about 12 weeks, about 24 weeks, or about 48 weeks from first administration of the composition.

In one embodiment, the subject exhibits an improvement in one or more of a metabolic syndrome, an adverse cardiovascular event, or diabetes mellitus.

In one embodiment, the metabolic syndrome is selected from waist circumference, obesity, hyperglycemia, dyslipidemia, and systemic hypertension (HTN).

In one embodiment, the subject exhibits no or substantially no weight gain, fluid retention, osteopenia, or increased fracture risk.

In one embodiment, the subject is heterozygous or homozygous for the PNPLA3 p.I148M allele (rs738409:G).

In one embodiment, the subject has high polygenic risk for cirrhosis. High polygenic risk can be in the top about 1%, about 2%, about 3%, about 4%, about 5%, about 10%, about 15%, about 20%, about 25%, about 30%, about 35%, or more polygenic risk.

In one embodiment, the subject has high polygenic risk for decompensated cirrhosis.

In one embodiment, the decompensated cirrhosis is jaundice, ascites, spontaneous bacterial peritonitis, variceal hemorrhage, hepatic encephalopathy, or hepatorenal syndrome.

In one embodiment, the subject has high polygenic risk for one or more of liver transplant, hepatocellular carcinoma, liver-related mortality, and end stage liver disease.

In one embodiment, the end stage liver disease is selected from the group consisting of decompensated cirrhosis, liver transplant, hepatopulmonary syndrome, complicated portal hypertension, hepatocellular carcinoma, or liver-related mortality.

One therapeutic use of the compounds or compositions of the present disclosure which inhibit HSD17B13 is to provide treatment to patients or subjects suffering primary sclerosing cholangitis, chronic liver disease, nonalcoholic fatty liver disease (NAFLD), nonalcoholic steatohepatitis (NASH), hepatitis C, alcoholic liver disease, liver damage due to progressive fibrosis, and liver fibrosis.

The disclosed compounds of the present disclosure can be administered in effective amounts to treat or prevent a disorder and/or prevent the development thereof in subjects.

Administration of the disclosed compounds can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a compound of the present disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564 which is hereby incorporated by reference in its entirety.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the Disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the present disclosure is directed to pharmaceutical compositions comprising a compound of Formula (I), Formula (II), or Formula (III) and a pharmaceutically acceptable carrier. The pharmaceutical acceptable carrier may further include an excipient, diluent, or surfactant. In some embodiments, the pharmaceutical composition can further comprise an additional pharmaceutically active agent.

In one embodiment, the pharmaceutical acceptable carrier further comprises an excipient, diluent, surfactant, or any combination thereof.

In one embodiment, the pharmaceutical composition further comprises at least one additional therapeutic agent.

Another aspect of the present disclosure is directed to pharmaceutical compositions for the treatment or prevention of a condition comprising a compound of Formula (I), Formula (II), or Formula (III), or a pharmaceutically acceptable salt, ester, or amino acid conjugate thereof.

In one embodiment, the condition is selected from primary sclerosing cholangitis, chronic liver disease, nonalcoholic fatty liver disease (NAFLD), nonalcoholic steatohepatitis (NASH), hepatitis C, alcoholic liver disease, liver damage due to progressive fibrosis, and liver fibrosis.

In one embodiment, the at least one additional therapeutic agent is selected from selonsertib, cenicriviroc, elafibrinor, obeticholic acid, or any combination thereof.

In one embodiment, the at least one additional therapeutic agent is selected from the group consisting of an farnesoid X receptor (FXR) activator, an acetyl-CoA carboxylase (ACC) inhibitor, an apoptosis signal-regulating kinase 1 (ASK1) inhibitor, PPAR alpha delta agonist, FGF19 agonist, thyroid beta agonist, FGF21 analog, ACC inhibitors, and a PNPLA3 modulator.

In one embodiment, the PNPLA3 modulator is selected from the group consisting of an antagonist, an inhibitor, a protein degrader, an RNA interference molecule, or an antisense oligonucleotide.

In one embodiment, the at least one additional therapeutic agent is selected from the group consisting of selonsertib, cenicriviroc, elafibrinor, obeticholic acid, firsocostat, and ciofexor.

In one embodiment, the pharmaceutical composition comprises no or substantially no amount of any additional therapeutic agent.

In one embodiment, the pharmaceutical composition lowers, decreases, and/or reduces one or more of hepatic steatosis, hepatic lipogenesis, bioactive lipid species formation, inhibition of fibrogenesis, lobular inflammation, portal inflammation, ballooning, NAFLD activity score, NASH category, fibrosis stage, alanine transaminase (ALT), aspartate aminotransferase (AST), gamma-glutamyl transferase (GGT), body weight, Enhanced Liver Fibrosis (ELF) score, Fibrosis-4 (FIB-4) index, N-terminal type III collagen propeptide (PRO-C3), interleukin 6 (IL-6), interleukin 8 (IL-8), interleukin 32 (IL-32), NIS-4 prognosis (alpha-2-macroglobulin (A2M), chitinase-3-like protein 1 (CHI3L1), hemoglobin A1c (HbA1c), and microRNA-34a (miR-34a)), cytokeratin-18 (CK-18), and magnetic resonance imaging corrected T1 (MRI cT).

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 1, 5, 20, 50, 75, 100, 150, 250, 500, 750, 1000, 1250, 2000, 2500, 3500, or 5000 mg of the disclosed compound, or, in a range of from one amount to another amount in the list of doses. In one embodiment, the compositions are in the form of a tablet that can be scored.

In one embodiment, the composition comprises about 1 mg to about 2000 mg of the compound.

In one embodiment, the composition is administered to the subject twice daily, once daily, once every other day, or once weekly.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Abbreviations used in the following examples and elsewhere herein are:
AcOH acetic acid
atm atmosphere
br broad
Amphos (4-(N,N-Dimethylamino)phenyl)di-tert-butyl phosphine
anh. anhydrous
aq. aqueous
BINAP (t)-2,2′-Bis(diphenylphosphino)-1,1′-binaphthalene
BrettPhos 2-(dicyclohexylphosphino)3,6-dimethoxy-2′,4′,6′-triisopropyl-1,1′-biphenyl
BrettPhos Pd G3 [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate
BuLi butyl lithium
DCM dichloromethane
DIAD diisopropyl azodiformate
DIEA N,N-diisopropylethylamine
DMAc N,N-dimethyl acetamide
DMAP N,N-dimethylpyridin-4-amine
DME 1,2-Dimethoxyethane
DMEDA N,N′-Dimethylethylenediamine
DMF N,N-dimethyl formamide
DMSO dimethyl sulfoxide
EDCI·HCl 1-(3-Dimethylaminopropyl)-3-ethylcarbodiimide Hydrochloride
EtOAc ethyl acetate
EtOH ethanol
h hour(s)
HBTU 3-[Bis(dimethylamino)methyliumyl]-3H-benzotriazol-1-oxide hexafluorophosphate
HPLC high pressure (or performance) liquid chromatography
KOtBu potassium tert-butoxide
LCMS liquid chromatography mass spectrometry
LHMDS Lithium bis(trimethylsilyl)amide
MeCN acetonitrile
2-MeTHF 2-methyl tetrahydrofuran
MeOH methanol
n-BuLi butyl lithium
NaOtBu sodium tert-butoxide
PEPPSI-iPr [1,3-Bis(2,6-Diisopropylphenyl)imidazol-2-ylidene](3-chloropyridyl)palladium(II) dichloride
PdCl₂(Amphos) Bis(di-tert-butyl(4-dimethylaminophenyl)phosphine)dichloropalladium(II)
Pd₂(dba)₃ Tris(dibenzylideneacetone)dipalladium(0)
Pd(OAc)₂ palladium(II) acetate
PdCl₂(dppf) [1,1′-Bis(diphenylphosphino)ferrocene]dichloropalladium(II)
quant. Quantitative
rac racemic mixture
rt room temperature
Rt retention time
sat. saturated
TBAB tetrabutylammonium bromide
TFA trifluoroacetic acid
THF tetrahydrofuran
XantPhos 4,5-Bis(diphenylphosphino)-9,9-dimethylxanthene
XPhos 2-Dicyclohexylphosphino-2′,4′,6′-triisopropylbiphenyl
ESI electrospray ionization
HATU [bis(dimethylamino)methylene]-1H-1,2,3-triazolo[4,5-b]pyridinium 3-oxide hexafluorophosphate
m multiplet
MeMgCl methylmagnesium chloride
MHz megahertz
min minutes
MS molecular sieves
MsCl methanesulfonyl chloride
MW microwave
NMR nuclear magnetic resonance
ppm parts per million
TLC thin layer chromatography
d doublet
t triplet
q quartet
L liter(s)
mL milliliter(s)
equiv equivalent(s)
g gram(s)
mg milligram(s)
mol mole(s)
mmol millimole(s)
M molar
Ar aryl
Het heteroaryl
Ph phenyl
A angstrom(s)
° C. degree(s) Celsius
TEA trimethylamine
CBz benzyl carbamate
TBD 1,5,7-triazabicyclo[4.4.0]dec-5-ene

The compounds of the present disclosure may be prepared by use of known chemical reactions and procedures. Nevertheless, the following general preparative methods are presented to aid the reader in synthesizing the HSD17B13 inhibitors with specific details provided below in the experimental section to illustrate working examples.

All variable groups of these methods are as described in the generic description if they are not specifically defined below.

It is recognized that compounds of the disclosure with each claimed optional functional group may not be prepared by each of the below-listed methods. Within the scope of each method, optional substituents may appear on reagents or intermediates which may act as protecting or otherwise non-participating groups. Utilizing methods well known to those skilled in the art, these groups are introduced and/or removed during the course of the synthetic schemes which provide the compounds of the present disclosure.

### Example 1: (Z)-5-(2,4,6-trifluoro-3-methoxybenzylidene)thiazolidine-2,4-dione (Intermediate I)

### Step 1: Synthesis of 2,4,6-trifluoro-3-methoxybenzaldehyde

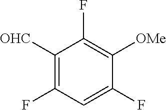 [SMILES: COc1c(F)cc(F)c(C=O)c1F]

To a stirred solution of 1,3,5-trifluoro-2-methoxybenzene (5.0 g, 30.86 mmol, 1.00 equiv) in THF (70.00 mL) at −78° C. was added dropwise a solution of n-BuLi (2.5 M in hexane, 13.6 mL, 33.95 mmol, 1.10 equiv) under N2 atmosphere. The resulting mixture was stirred at this temperature for 0.5 h and to this was added DMF (6.85 g, 92.59 mmol, 3.00 equiv). The resulting mixture was stirred for another 1 h and then quenched with NH₄Cl solution and extracted EtOAc 3 times. The combined organic layers were dried over Na₂SO₄ and concentrated under vacuum. The residue was purified by flash chromatography on silica gel (0-30% EtOAc in petroleum ether) to afford 2,4,6-trifluoro-3-methoxybenzaldehyde (4.2 g, 71%) as a yellow solid.

### Step 2: Synthesis of (Z)-5-(2,4,6-trifluoro-3-methoxybenzylidene)thiazolidine-2,4-dione

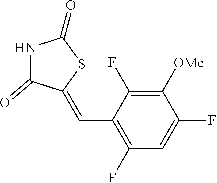 [SMILES: COc1c(F)cc(F)c(/C=C2\SC(=O)NC2=O)c1F]

A mixture of thiazolidine-2,4-dione (2 g, 17.09 mmol, 1.0 equiv), 2,4,6-trifluoro-3-methoxybenzaldehyde (3.25 g, 17.09 mmol, 1.00 equiv), piperidine (0.15 g, 1.7 mmol, 0.10 equiv) and AcOH (0.1 g, 0.1 mmol, 0.10 equiv) in toluene (50.00 mL) was stirred at 110° C. for 48 h and then concentrated under vacuum. The residue was purified by flash chromatography on silica gel (0-80% EtOAc in petroleum ether) to afford (Z)-5-(2,4,6-trifluoro-3-methoxybenzylidene)thiazolidine-2,4-dione (2.6 g, 51%) as a yellow solid. ¹H NMR (300 MHz, DMSO-d₆) δ 12.72 (brs, 1H), 7.56-7.19 (m, 2H), 3.92 (s, 3H). MS (ESI, m/z): 288 (M−H)⁺.

### Example 2: (Z)-5-(4-fluoro-3-methoxybenzylidene)thiazolidine-2,4-dione (Intermediate II)

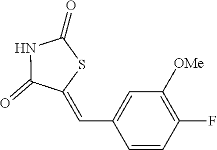 [SMILES: COc1cc(/C=C2\SC(=O)NC2=O)ccc1F]

A mixture of thiazolidine-2,4-dione (1.67 g, 14.270 mmol, 1.10 equiv), 4-fluoro-3-methoxybenzaldehyde (2.00 g, 12.980 mmol, 1.00 equiv), piperidine (110.00 mg, 1.290 mmol, 0.10 equiv) and AcOH (60.05 mg, 1.290 mmol, 0.10 equiv) in toluene (50.00 mL) was stirred at reflux for 16 h and then concentrated under vacuum. The residue was purified by flash chromatography on silica gel (0-80% EtOAc in petroleum ether) to afford (Z)-5-(4-fluoro-3-methoxybenzylidene) thiazolidine-2,4-dione (1.84 g, 94.2%) as a brown solid. ¹H NMR (300 MHz, DMSO-d₆) δ 12.63 (s, 1H), 7.78 (s, 1H), 7.46-7.32 (m, 2H), 7.21-7.12 (m, 1H), 3.89 (s, 3H). MS (ESI, m/z): 254 (M+H)⁺.

### Example 3: General Procedure I

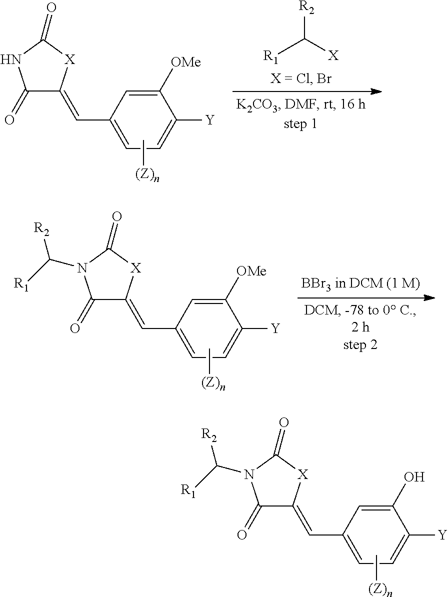

### Step 1: (Z)-3-((aryl methyl)-5-(2,4,6-trifluoro-3-methoxybenzylidene)thiazolidine-2,4-dione

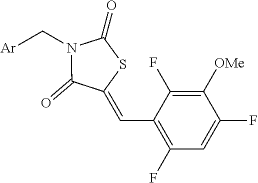

A mixture of corresponding aryl methyl halide (1.00 equiv), (Z)-5-(2,4,6-trifluoro-3-methoxybenzylidene)thiazolidine-2,4-dione (1.00 equiv) and K₂CO₃ (576.15 mg, 4.169 mmol, 3 equiv) in DMF (5 mL) was stirred at ambient temperature for 16 h and then diluted with EtOAc. The organic layer was washed with brine 3 times, dried over Na₂SO₄ and concentrated under vacuum. The residue was purified by flash chromatography on silica gel or reverse phase flash chromatography on C18 gel to afford (Z)-3-((aryl methyl)-5-(2,4,6-trifluoro-3-methoxybenzylidene)thiazolidine-2,4-dione (113 mg, 19.82%) as a yellow oil.

### Step 2: (Z)-3-(aryl methyl)-5-(2,4,6-trifluoro-3-hydroxybenzylidene)thiazolidine-2,4-dione

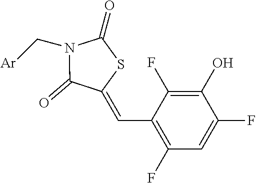

To a stirred solution of 3-((aryl methyl)-5-(2,4,6-trifluoro-3-methoxybenzylidene)thiazolidine-2,4-dione (1.00 equiv) in DCM (5 mL) at −78° C. was added dropwise BBr₃ (1 M in DCM, 0.83 mL, 0.83 mmol, 3.00 equiv) under N2 atmosphere. The resulting mixture was allowed to warm up to ambient temperature and stirred for another 16 h. The resulting mixture was quenched with MeOH at 0° C. and then concentrated under vacuum. The residue was purified by reverse phase chromatography or Prep-HPLC to afford (Z)-3-(aryl methyl)-5-(2,4,6-trifluoro-3-hydroxybenzylidene)thiazolidine-2,4-dione.

### Example 4: General Procedure H

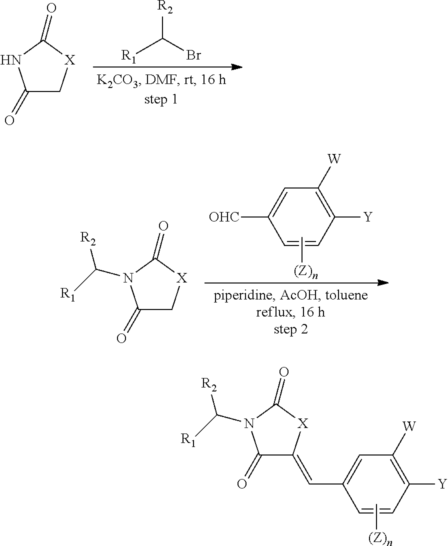

### Step 1: 3-((aryl/heterocyclic)methyl)thiazolidine-2,4-dione

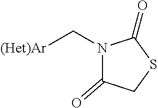

A mixture of thiazolidine-2,4-dione (1 equiv), corresponding aryl/heterocyclic methylhalidel-(bromomethyl)-3-(methylsulfonyl)benzene (1 equiv) and K₂CO₃ (3 equiv) in DMF (0.5 g/10 mL) was stirred at ambient temperature for 16 h, and then diluted with EtOAc. The resulting mixture was washed with brine 3 times, dried over Na₂SO₄ and concentrated under vacuum. The residue was purified by flash chromatography to afford 3-((aryl/heterocyclic)methyl)thiazolidine-2,4-dione.

### Step 2: (Z)-3-((aryl/heterocyclic)methyl)-5-(2,4,6-trifluoro-3-hydroxybenzylidene)thiazolidine-2,4-dione

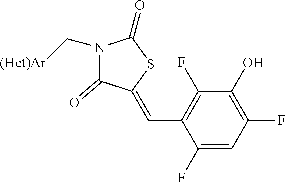

A mixture of 3-((aryl/heterocyclic)methyl)thiazolidine-2,4-dione (1 equiv), 2,4,6-trifluoro-3-hydroxybenzaldehyde (1.2 equiv), AcOH 0.2 equiv) and pyridine (0.2 equiv) in toluene (150 mg/5 mL) was stirred at reflux for 16 h and then concentrated under vacuum. The residue was purified by reverse phase flash chromatography on C18 gel to afford (Z)-3-((aryl/heterocyclic)methyl)-5-(2,4,6-trifluoro-3-hydroxybenzylidene)thiazolidine-2,4-dione.

### Example 5: General Procedure III

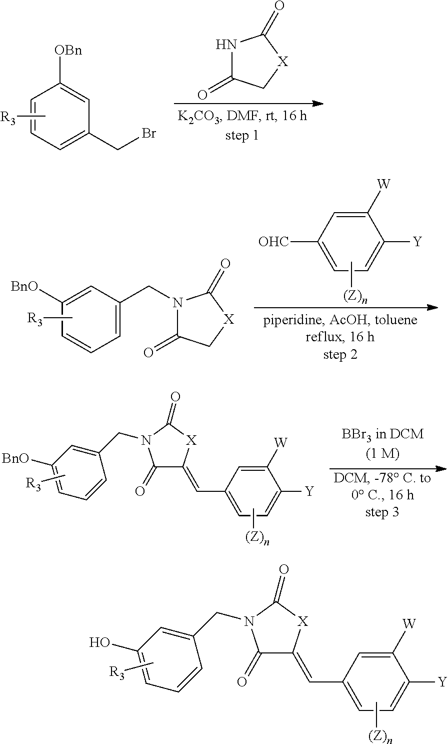

### Step 1: 3-(3-(benzyloxy)benzyl)thiazolidine-2,4-dione

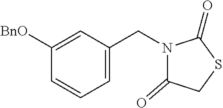 [SMILES: O=C1CSC(=O)N1Cc1cccc(OCc2ccccc2)c1]

To a stirred solution of 1-(benzyloxy)-3-(bromomethyl)benzene (500 mg, 1.680 mmol, 1.10 equiv) and thiazolidine-2,4-dione (192 mg, 1.640 mmol, 1.00 equiv) in DMF (10.00 mL) at ambient temperature was added K₂CO₃ (680 mg, 4.920 mmol, 3.00 equiv). The resulting mixture was stirred at this temperature for 16 h, and then diluted with EtOAc. The organic mixture was washed with brine 3 times, dried over Na₂SO₄ and concentrated under vacuum. The residue was purified by flash chromatography on silica gel (0-30% EtOAc in petroleum ether) to afford 3-(3-(benzyloxy)benzyl)thiazolidine-2,4-dione (323 mg, 96%) as colorless oil. ¹H NMR (300 MHz, DMSO-d₆) δ 7.58-7.18 (m, 6H), 7.01-6.77 (m, 3H), 5.07 (s, 2H), 4.64 (s, 2H), 4.28 (s, 2H).

### Step 2: (Z)-5-((aryl/hetercycilic)methylene)-3-(3-(benzyloxy)benzyl)thiazolidine-2,4-dione

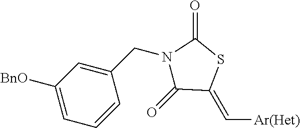

A mixture of 3-(3-(benzyloxy)benzyl)thiazolidine-2,4-dione (1.00 equiv), corresponding aryl/hetercyclic aldehyde (1.30 equiv), piperidine (0.20 equiv) and AcOH (0.20 equiv) in toluene (150 mg/10.00 mL) was stirred at reflux for 16 h and then concentrated under vacuum to afford (Z)-5-((aryllhetercyclic)methylene)-3-(3-(benzyloxy)benzyl)thiazolidine-2,4-dione (crude), which was used for the next step without further purification.

### Step 3: (Z)-5-((aryl/hetercyclic)methylene)methylene)-3-(3-hydroxybenzyl)thiazolidine-2,4-dione

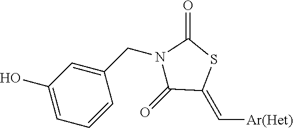

To a stirred solution of (Z)-5-((aryl/hetercyclic)methylene)-3-(3-(benzyloxy)benzyl)thiazolidine-2,4-dione (1.00 equiv) in DCM (200 mg/5.00 mL) at −78° C. was added dropwise a solution of BBr₃ (1 M in DCM, 6 equiv). The resulting mixture was allowed to warm up to ambient temperature and stirred for another 16 h and then quenched with MeOH at 0° C. The resulting mixture was concentrated under vacuum. The residue was purified by reverse flash chromatography on C18 gel to afford (Z)-5-((aryl/hetercyclic)methylene)methylene)-3-(3-hydroxybenzyl)thiazolidine-2,4-dione.

### Example 6: General Procedure IV

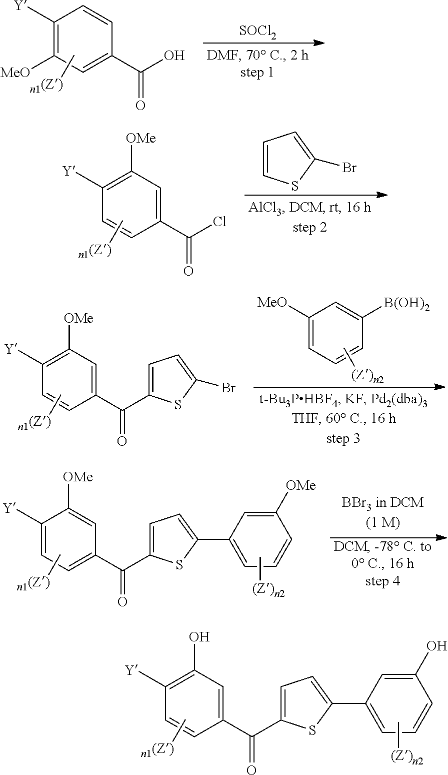

### Step 1: Substituted-3-methoxybenzoyl Chloride

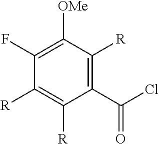

A solution of corresponding 3-methoxybenzoic acid (1.00 equiv) in SOCl₂ (300 mg/i mL) was stirred at reflux for 2 h and then concentrated under vacuum to afford substituted-3-methoxybenzoyl chloride, which was used for the next step without further purification.

### Step 2: (5-bromothiophen-2-yl)(substituted-3-methoxyphenyl)methanone

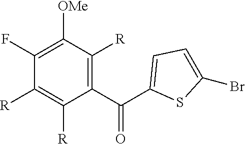

To a stirred solution of substituted 3-methoxybenzoyl chloride (1.00 equiv) and 2-bromothiophene (1.30 equiv) in DCM (300 mg/5 mL) at 0° C. was added AlCl₃ (2.00 equiv) in several portions. The resulting reaction mixture was stirred at ambient temperature for 16 h and then quenched with water and extracted with EtOAc 3 times. The organic layers were combined, dried over Na₂SO₄ and concentrated under vacuum. The residue was purified by flash chromatography on silica gel to afford (5-bromothiophen-2-yl)(substituted-3-methoxyphenyl)methanone.

### Step 3: (5-(2,4-difluoro-3-methoxyphenyl)thiophen-2-yl)(substituted-3-methoxyphenyl)methanone

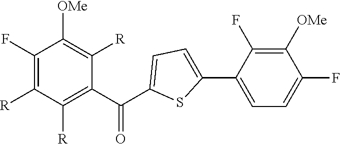

A mixture of (5-bromothiophen-2-yl)(substituted-3-methoxyphenyl)methanone (1.00 equiv), 2,4-difluoro-3-methoxyphenylboronic acid (1.50 equiv), KF (5.00 equiv), t-BuP·HBF₄ (0.20 equiv) and Pd₂(dba)₃ (0.20 equiv) in THF (100 mg/5 mL) was stirred at 60° C. for 16 h under N₂ atmosphere. The reaction mixture was diluted with EtOAc and washed with bine 3 times. The organic layer was dried over Na₂SO₄ and concentrated under vacuum. The residue was purified by flash chromatography on silica gel to afford (5-(2,4-difluoro-3-methoxyphenyl)thiophen-2-yl)(substituted-3-methoxyphenyl)methanone.

### Step 4: (5-(2,4-difluoro-3-hydroxyphenyl)thiophen-2-yl)(substituted 3-hydroxyphenyl)methanone

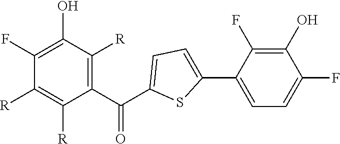

To a stirred solution of (5-(2,4-difluoro-3-methoxyphenyl)thiophen-2-yl)(substituted-3-methoxyphenyl)methanone (1.00 equiv) in DCM (100 mg/5.00 mL) at −78° C. was added dropwise BBr₃ (1 M in DCM, 2.11 mL, 2.110 mmol, 6.00 equiv, 1 M). The resulting solution was stirred at ambient temperature for 16 h and then quenched with MeOH at 0° C. and concentrated under vacuum. The residue was purified by flash chromatography on silica gel to afford (5-(2,4-difluoro-3-hydroxyphenyl)thiophen-2-yl)(submitted-3-hydroxyphenyl)methanone.

### Example 7: General Procedure V

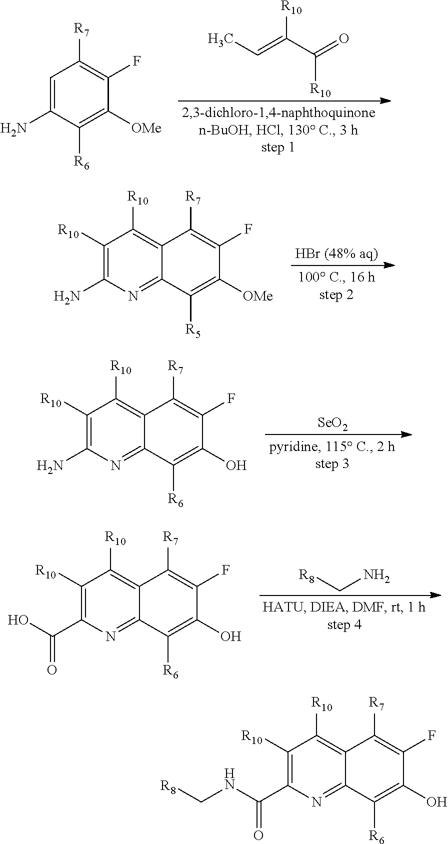

### Step 1: 6-fluoro-7-methoxy-2-methylquinoline

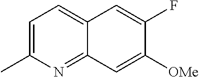

To a stirred solution of 4-fluoro-3-methoxyaniline (1.00 g, 7.09 mmol, 1.00 equiv) in n-BuOH (3.00 mL) at 0° C. was added dropwise HCl (12 M, 2.5 mL). The resulting mixture was stirred at this temperature for 10 mins and to this was added 2,3-dichloro-1,4-naphthoquinone (1.77 g, 7.80 mmol, 1.10 equiv). The resulting solution was warmed up to 130° C. and to this was added dropwise a solution of (E)-but-2-enal (744.88 mg, 10.63 mmol, 1.50 equiv) in n-BuOH (2.00 mL) over 30 mins. The resulting mixture was stirred at this temperature for another 2 h and then neutralized with NaOH solution to pH 10 and extracted with EtOAc 3 times. The organic layers were combined, dried over Na₂SO₄, and concentrated under vacuum. The residue was purified by reverse phase flash chromatography on C18 gel (5-80% acetonitrile in water) to afford 6-fluoro-7-methoxy-2-methylquinoline (600 mg, 44.29%) as brown oil. MS (ESI, m % z): 191 (M+H).

### Step 2: 6-fluoro-2-methylquinolin-7-ol

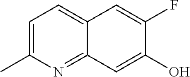

A mixture of 6-fluoro-7-methoxy-2-methylquinoline (600 mg, 3.14 mmol, 1.00 equiv) in HBr (48% solution, 10 mL) was stirred at 100° C. for 16 h and then concentrated under vacuum. The residue was purified by revers phase flash chromatography on C18 gel (5-80% acetonitrile in water) to afford 6-fluoro-2-methylquinolin-7-ol (410 mg, 74%) as a brown solid. MS (ESI, m/z): 178 (M+H)⁺.

### Step 3: 6-fluoro-7-hydroxyquinoline-2-carboxylic Acid

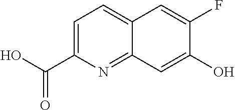 [SMILES: O=C(O)c1ccc2cc(F)c(O)cc2n1]

A mixture of 6-fluoro-2-methylquinolin-7-ol (410 mg, 2.32 mmol, 1.00 equiv) and SeO₂ (283 mg, 2.55 mmol, 1.1 equiv) in pyridine (10.00 mL) was stirred at 115° C. for 2 h and then concentrated under vacuum. The residue was diluted with water and filtered. The filtrate was concentrated to afford 6-fluoro-7-hydroxyquinoline-2-carboxylic acid (crude, 380 mg) as a brown solid, which was used for the next step without further purification. MS (EST, m/z): 208 (M+H)⁺.

### Step 4: 6-fluoro-7-hydroxy-N-(3-aryl)quinoline-2-carboxamide

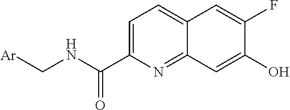

To a stirred mixture of 6-fluoro-7-hydroxyquinoline-2-carboxylic acid (1.00 equiv), DIEA (3.00 equiv) and corresponding aryl methanamine (30 mg, 0.242 mmol, 1.00 equiv) in DMF (50 mg/6.00 mL) at ambient temperature was added HATU (138 mg, 0.362 mmol, 1.50 equiv). The resulting mixture was stirred at this temperature for 1 h and then purified directly by reverse phase flash chromatography on C18 gel to afford 6-fluoro-7-hydroxy-N-(3-aryl)quinoline-2-carboxamide.

### Example 8: (Z)-3-(3-(methylsulfonyl)benzyl)-5-(2,4,6-trifluoro-3-hydroxybenzylidene) thiazolidine-2,4-dione (Compound 5)

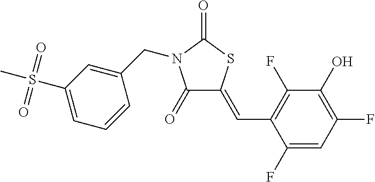 [SMILES: CS(=O)(=O)c1cccc(CN2C(=O)S/C(=C\c3c(F)cc(F)c(O)c3F)C2=O)c1]

The product was synthesized following General Procedure II starting from 1-(bromomethyl)-3-(methylsulfonyl)benzene to afford (Z)-3-(3-(methylsulfonyl)benzyl)-5-(2,4,6-trifluoro-3-hydroxybenzylidene)thiazolidine-2,4-dione (27.3 mg) as a white solid. ¹H NMR (300 MHz, DMSO-d₆) δ 10.64 (brs, 1H), 7.93-7.84 (m, 2H), 7.76 (s, 1H), 7.73-7.59 (m, 2H), 7.45-7.31 (m, 1H), 4.93 (s, 2H), 3.23 (s, 1H). MS (ESI, m/z): 442 (M−H)⁻.

### Example 9: (Z)-5-((1H-benzo[d][1,2,3]triazol-6-yl)methylene)-3-(3-hydroxybenzyl)thiazolidine-2,4-dione (Compound 38)

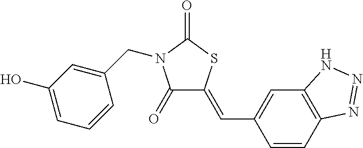 [SMILES: O=C1S/C(=C\c2ccc3nn[nH]c3c2)C(=O)N1Cc1cccc(O)c1]

The product was synthesized following General Procedure III and 1H-benzo[d][1,2,3]triazole-6-carbaldehyde to afford (Z)-5-((1H-benzo[d][1,2,3]triazol-6-yl)methylene)-3-(3-hydroxybenzyl)thiazolidine-2,4-dione (115 mg) as a light yellow solid. ¹H NMR (300 MHz, DMSO-d₆) δ 9.52 (brs, 1H), 8.27 (s, 1H), 8.20 (s, 1H), 8.06 (d, J=8.7 Hz, 1H), 7.71 (dd, J=8.7, 1.5 Hz, 1H), 7.15 (t, J=7.8 Hz, 1H), 6.79-6.64 (m, 3H), 4.78 (s, 2H). MS (ESI, m/z): 353 (M+H)⁺.

### Example 10: (Z)-3-((2-methoxypyridin-4-yl)methyl)-5-(2,4,6-trifluoro-3-hydroxybenzylidene) thiazolidine-2,4-dione (Compound 22)

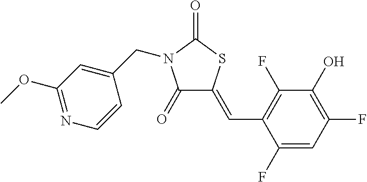 [SMILES: COc1cc(CN2C(=O)S/C(=C\c3c(F)cc(F)c(O)c3F)C2=O)ccn1]

The product was synthesized following General Procedure II starting from 4-(chloromethyl)-2-methoxypyridine to afford (Z)-3-((2-methoxypyridin-4-yl)methyl)-5-(2,4,6-trifluoro-3-hydroxybenzylidene)thiazolidine-2,4-dione (33.1 mg) as a white solid. ¹H NMR (300 MHz, DMSO-d₆) δ 10.72 (s, 1H), 8.14 (t, J=5.4 Hz, 1H), 7.75 (s, 1H), 7.43-7.36 (m, 1H), 6.91 (dd, J=5.4, 1.5 Hz, 1H), 6.74 (s, 1H), 4.81 (s, 2H), 3.84 (s, 3H). MS (ESI, m/z): 397 (M+H)⁺.

### Example 11: (Z)-3-((2-methoxypyridin-4-yl)methyl)-5-(2,4,6-trifluoro-3-hydroxybenzylidene) thiazolidine-2,4-dione

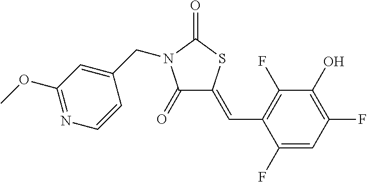 [SMILES: COc1cc(CN2C(=O)S/C(=C\c3c(F)cc(F)c(O)c3F)C2=O)ccn1]

The product was synthesized following General Procedure I starting from Intermediate I and 4-(chloromethyl)-2-methoxypyridine to afford (Z)-3-((2-methoxypyridin-4-yl)methyl)-5-(2,4,6-trifluoro-3-hydroxybenzylidene)thiazolidine-2,4-dione (92 mg) as a yellow solid. MS (ESI, m/z): 397 (M+H)⁺

### Example 12: (Z)-3-((5-(4-fluoro-3-hydroxybenzylidene)-2,4-dioxothiazolidin-3-yl)methyl) benzenesulfonamide (Compound 33)

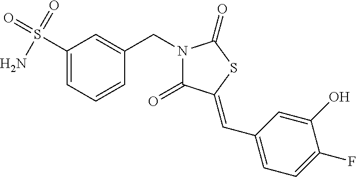 [SMILES: NS(=O)(=O)c1cccc(CN2C(=O)S/C(=C\c3ccc(F)c(O)c3)C2=O)c1]

The product was synthesized following General Procedure I starting from Intermediate II and 3-(bromomethyl)benzenesulfonamide to afford (Z)-3-((5-(4-fluoro-3-hydroxybenzylidene)-2,4-dioxothiazolidin-3-yl)methyl)benzenesulfonamide (64.9 mg) as a white solid. ¹H NMR (300 MHz, DMSO-d₆) δ 10.39 (s, 1H), 7.89 (s, 1H), 7.81-7.72 (m, 2H), 7.63-7.51 (m, 2H), 7.39 (s, 2H), 7.33 (dd, J=11.1, 8.4 Hz, 1H), 7.24 (dd, J=8.4, 2.4 Hz, 1H), 7.18-7.09 (m, 1H), 4.91 (s, 2H). MS (ESI, m/Z): 409 (M+H)⁺.

### Example 13: (Z)-3-((2,4-dioxo-5-(2,4,6-trifluoro-3-hydroxybenzylidene)thiazolidin-3-yl)methyl)benzenesulfonamide (Compound 34)

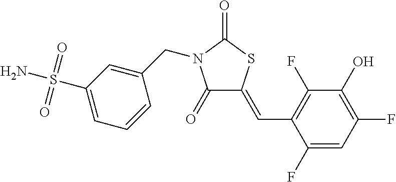 [SMILES: NS(=O)(=O)c1cccc(CN2C(=O)S/C(=C\c3c(F)cc(F)c(O)c3F)C2=O)c1]

The product was synthesized following General Procedure I starting from Intermediate I and 3-(bromomethyl)benzenesulfonamide to afford (Z)-3-((2,4-dioxo-5-(2,4,6-trifluoro-3-hydroxybenzylidene)thiazolidin-3-yl)methyl)benzenesulfonamide (16.1 mg) as a yellow solid. ¹H NMR (300 MHz, DMSO-d₆) δ 10.57 (s, 1H), 7.83-7.72 (m, 3H), 7.63-7.50 (m, 2H), 7.46-7.32 (m, 3H), 4.90 (s, 2H). MS (ESI, m/z): 445 (M+H)⁺.

### Example 14: (Z)-3-((6-methoxypyridin-3-yl)methyl)-5-(2,4,6-trifluoro-3-hydroxybenzylidene) thiazolidine-2,4-dione (Compound 23)

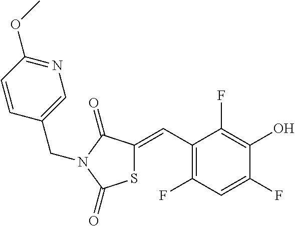 [SMILES: COc1ccc(CN2C(=O)S/C(=C\c3c(F)cc(F)c(O)c3F)C2=O)cn1]

The product was synthesized following General Procedure I starting from Intermediate I and 5-(chloromethyl)-2-methoxypyridine to afford (Z)-3-((6-methoxypyridin-3-yl)methyl)-5-(2,4,6-trifluoro-3-hydroxybenzylidene)thiazolidine-2,4-dione (186 mg) as yellow solid. MS (ESI, m/z): 397 (M+H)⁺

### Example 15: (Z)-3-((6-methoxypyridin-3-yl)methyl)-5-(2,4,6-trifluoro-3-hydroxybenzylidene)thiazolidine-2,4-dione (Compound 23)

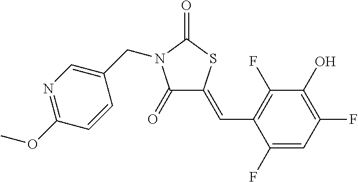 [SMILES: COc1ccc(CN2C(=O)S/C(=C\c3c(F)cc(F)c(O)c3F)C2=O)cn1]

The product was synthesized following General Procedure II starting from 5-(chloromethyl)-2-methoxypyridine to afford (Z)-3-((6-methoxypyridin-3-yl)methyl)-5-(2,4,6-trifluoro-3-hydroxybenzylidene)thiazolidine-2,4-dione (51.1 mg) as a yellow solid. ¹H NMR (300 MHz, DMSO-d₆) δ 8.16 (d, J=2.4 Hz, 1H), 7.74 (s, 1H), 7.64 (dd, J=8.4, 2.4 Hz, 1H), 7.08-6.95 (m, 1H), 6.84-6.75 (m, 1H), 4.75 (s, 2H). MS (ESI, m/z): 397 (M+H)⁺.

### Example 16: (Z)-3-(3-hydroxybenzyl)-5-(2,4,6-trifluoro-3-hydroxybenzylidene)thiazolidine-2,4-dione (Compound 4)

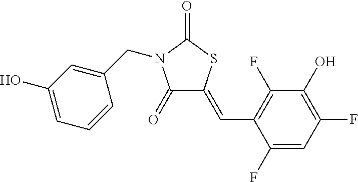 [SMILES: O=C1S/C(=C\c2c(F)cc(F)c(O)c2F)C(=O)N1Cc1cccc(O)c1]

The product was synthesized following General Procedure III and 2,4,6-trifluoro-3-methoxybenzaldehyde to afford (Z)-3-(3-hydroxybenzyl)-5-(2,4,6-trifluoro-3-hydroxybenzylidene)thiazolidine-2,4-dione (22.4 mg) as a yellow solid. ¹H NMR (400 MHz, DMSO-d₆) δ 9.53 (brs, 1H), 7.76 (s, 1H), 7.34 (t, J=10.4 Hz, 1H), 7.18-7.10 (m, 1H), 6.75-6.65 (m, 3H), 4.74 (s, 2H). MS (ESI, m/z): 382 (M+H)⁺

### Example 17: (5-(2,4-difluoro-3-hydroxyphenyl)thiophen-2-yl)(4-fluoro-3-hydroxyphenyl) methanone (Compound 28)

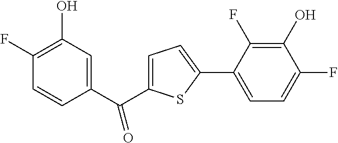 [SMILES: O=C(c1ccc(F)c(O)c1)c1ccc(-c2ccc(F)c(O)c2F)s1]

The product was synthesized following General Procedure IV starting from 4-fluoro-3-methoxybenzoic acid to afford (5-(2,4-difluoro-3-hydroxyphenyl)thiophen-2-yl)(4-fluoro-3-hydroxyphenyl)methanone (29.6 mg) as a yellow solid. ¹H NMR (300 MHz, DMSO-d₆) δ 10.51 (s, 2H), 7.76 (dd, J=4.2, 1.2 Hz, 1H), 7.67 (d, J=4.2 Hz, 1H), 7.50-7.41 (m, 1H), 7.41-7.27 (m, 3H), 7.24-7.11 (m, 1H). MS (ESI, m/z): 351 (M+H)⁺.

### Example 18: (Z)-3-(3-(1H-1,2,3-triazol-4-yl)benzyl)-5-(4-fluoro-3-hydroxybenzylidene) thiazolidine-2,4-dione (Compound 15)

### Step 1: (Z)-5-(4-fluoro-3-methoxybenzylidene)-3-(3-iodobenzyl)thiazolidine-2,4-dione

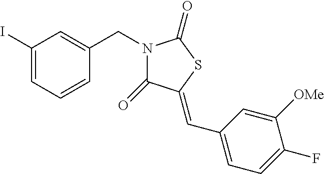 [SMILES: COc1cc(/C=C2\SC(=O)N(Cc3cccc(I)c3)C2=O)ccc1F]

A mixture of (Z)-5-(4-fluoro-3-methoxybenzylidene)thiazolidine-2,4-dione (0.8 g, 3.159 mmol, 1.00 equiv), 1-(bromomethyl)-3-iodobenzene (1.41 g, 4.738 mmol, 1.50 equiv) and K₂CO₃ (1.31 g, 9.477 mmol, 3.00 equiv) in DMF (5 mL) was stirred at ambient temperature for 16 h. The reaction mixture was diluted with EtOAc, washed with brine 3 times, dried over Na₂SO₄ and concentrated under vacuum. The residue was purified by flash chromatography on silica gel (0-40% EtOAc in petroleum ether) to afford (Z)-5-(4-fluoro-3-methoxybenzylidene)-3-(3-iodobenzyl)thiazolidine-2,4-dione (1.368 g, 84%)) as a yellow solid. MS (ESI, m/z): 470 (M+H)⁺.

### Step 2: (Z)-5-(4-fluoro-3-methoxybenzylidene)-3-(3-((trimethylsilyl)ethynyl) benzyl)thiazolidine-2,4-dione

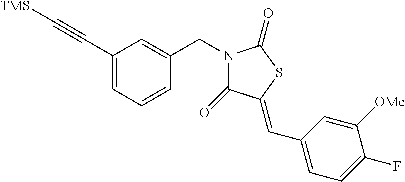 [SMILES: COc1cc(/C=C2\SC(=O)N(Cc3cccc(C#C[Si](C)(C)C)c3)C2=O)ccc1F]

A mixture of (Z)-5-(4-fluoro-3-methoxybenzylidene)-3-(3-iodobenzyl)thiazolidine-2,4-dione (0.8 g, 1.705 mmol, 1.00 equiv), ethynyltrimethylsilane (251 mg, 2.557 mmol, 1.50 equiv), CuI (32 mg, 0.170 mmol, 0.10 equiv) and Pd(PPh₃)₂Cl₂ (120 mg, 0.170 mmol, 0.10 equiv) in TEA (5.00 mL) and THF (5.00 mL) was stirred at 40° C. for 16 h under N2 atmosphere and then concentrated under vacuum. The residue was purified by flash chromatography on silica gel (0-40% EtOAc in petroleum ether) to afford (Z)-5-(4-fluoro-3-methoxybenzylidene)-3-(3-((trimethylsilyl)ethynyl)benzyl)thiazolidine-2,4-dione (265 mg, 33%) as a yellow solid. MS (ESI, m/z): 440 (M+H)⁺.

### Step 3: (Z)-3-(3-ethynylbenzyl)-5-(4-fluoro-3-methoxybenzylidene)thiazolidine-2,4-dione

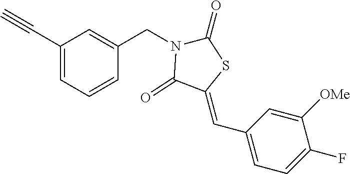 [SMILES: C#Cc1cccc(CN2C(=O)S/C(=C\c3ccc(F)c(OC)c3)C2=O)c1]

A mixture of (Z)-5-(4-fluoro-3-methoxybenzylidene)-3-(3-((trimethylsilyl)ethynyl)benzyl)thiazolidine-2,4-dione (265.00 mg, 0.603 mmol, 1.00 equiv) and K₂CO₃ (167 mg, 1.206 mmol, 2.00 equiv) in MeOH (10 mL) was stirred at ambient temperature for 1 h and then concentrated under vacuum. The residue was purified by flash chromatography on silica gel (0-30% EtOAc in petroleum ether) to afford (Z)-3-(3-ethynylbenzyl)-5-(4-fluoro-3-methoxybenzylidene)thiazolidine-2,4-dione (76 mg, 34%) as a yellow solid. MS (ESI, m/z): 368 (M+H)⁺.

### Step 4: (Z)-3-(3-(1H-1,2,3-triazol-4-yl)benzyl)-5-(4-fluoro-3-methoxybenzylidene) thiazolidine-2,4-dione

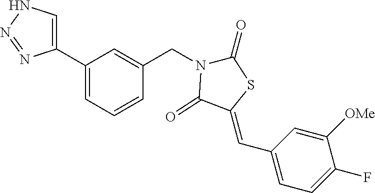 [SMILES: COc1cc(/C=C2\SC(=O)N(Cc3cccc(-c4c[nH]nn4)c3)C2=O)ccc1F]

A mixture of(Z)-3-(3-ethynylbenzyl)-5-(4-fluoro-3-methoxybenzylidene)thiazolidine-2,4-dione (220.00 mg, 0.599 mmol, 1.00 equiv), azidotrimethylsilane (207 mg, 1.796 mmol, 3.00 equiv) and CuI (23 mg, 0.120 mmol, 0.20 equiv) in MeOH (1.00 mL) and DMF (10.00 mL) was stirred at 100° C. for 24 h under N2 atmosphere. The reaction mixture was diluted with EtOAc, washed with brine 3 times, dried over Na₂SO₄ and concentrated under vacuum. The residue was purified by reverse phase flash chromatography on C18 gel (5-60% acetonitrile in water) to afford (Z)-3-(3-(1H-1,2,3-triazol-4-yl)benzyl)-5-(4-fluoro-3-methoxybenzylidene)thiazolidine-2,4-dione (60 mg, 19%) as a yellow solid. MS (ESI, m/z): 411 (M+H)⁺.

### Step 5: (Z)-3-(3-(1H-1,2,3-triazol-4-yl)benzyl)-5-(4-fluoro-3-hydroxybenzylidene) thiazolidine-2,4-dione (Compound 15)

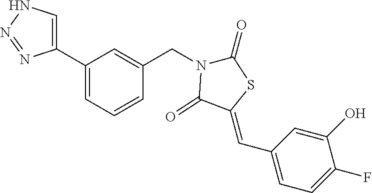 [SMILES: O=C1S/C(=C\c2ccc(F)c(O)c2)C(=O)N1Cc1cccc(-c2c[nH]nn2)c1]

To a stirred solution of (Z)-3-(3-(1H-1,2,3-triazol-4-yl)benzyl)-5-(4-fluoro-3-methoxybenzylidene)thiazolidine-2,4-dione (60.00 mg, 0.146 mmol, 1.00 equiv) in DCM (10.00 mL) at −78° C. was added dropwise a solution of BBr₃ (1 M in DCM, 0.88 mL, 0.880 mmol, 6.02 equiv,) over 5 mins. The resulting reaction mixture was allowed to warm up to ambient temperature and then stirred for another 2 h. The resulting mixture was quenched with MeOH at 0° C. and then concentrated under vacuum. The residue was purified by reverse phase flash chromatography on C18 gel (5-100% acetonitrile in water) to afford (Z)-3-(3-(1H-1,2,3-triazol-4-yl)benzyl)-5-(4-fluoro-3-hydroxybenzylidene)thiazolidine-2,4-dione (8 mg, 13%) as a yellow solid. ¹H NMR (300 MHz, DMSO-d₆) δ10.39 (s, 1H), 8.34 (brs, 1H), 7.89 (s, 1H), 7.86-7.74 (m, 2H), 7.44 (t, J=7.8 Hz, 1H), 7.39-7.19 (m, 3H), 7.15 (d, J=4.2 Hz, 1H), 4.89 (s, 2H). MS (ESI, m/z): 397 (M+H)⁺.

### Example 19: (Z)-5-(4-fluoro-3-hydroxybenzylidene)-3-(3-(methylsulfonyl)benzyl)thiazolidine-2,4-dione (Compound 12)

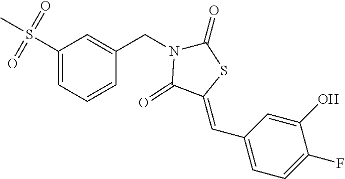 [SMILES: CS(=O)(=O)c1cccc(CN2C(=O)S/C(=C\c3ccc(F)c(O)c3)C2=O)c1]

The product was synthesized following General Procedure I starting from Intermediate II and 1-(bromomethyl)-3-(methylsulfonyl)benzene to afford (Z)-5-(4-fluoro-3-hydroxybenzylidene)-3-(3-(methylsulfonyl)benzyl)thiazolidine-2,4-dione (32 mg) as a white solid. ¹H NMR (300 MHz, DMSO-d₆) δ 10.38 (s, 1H), 7.92-7.83 (m, 3H), 7.71-7.58 (m, 2H), 7.38-7.27 (m, 1H), 7.26-7.18 (m, 1H), 7.18-7.07 (m, 1H), 4.94 (s, 2H), 3.22 (s, 3H). MS (ESI, m/z): 408 (M+H)⁺.

### Example 20: (5-(2,4-difluoro-3-hydroxyphenyl)thiophen-2-yl)(2,4,6-trifluoro-3-hydroxyphenyl)methanone (Compound 29)

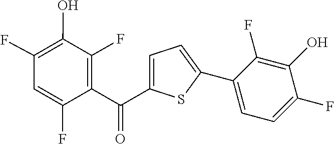 [SMILES: O=C(c1ccc(-c2ccc(F)c(O)c2F)s1)c1c(F)cc(F)c(O)c1F]

The product was synthesized following General Procedure IV starting from 2,4,6-trifluoro-3-methoxybenzoic acid to afford (5-(2,4-difluoro-3-hydroxyphenyl)thiophen-2-yl)(2,4,6-trifluoro-3-hydroxyphenyl)methanone (71.3 mg) as an off-white solid. ¹H NMR (300 MHz, DMSO-d₆) δ 10.64 (s, 2H), 7.78-7.72 (m, 1H), 7.70-7.63 (m, 1H), 7.47-7.31 (m, 2H), 7.24-7.14 (m, 1H). MS (ESI, m/z) 387 (M+H)⁺.

### Example 21: (5-(2,4-difluoro-3-hydroxyphenyl)thiophen-2-yl)(3,4-difluoro-5-hydroxyphenyl) methanone (Compound 30)

### Step 1: 3,4-difluoro-N,5-dimethoxy-N-methylbenzamide

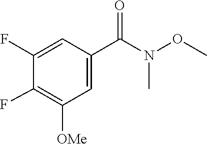 [SMILES: COc1cc(C(=O)N(C)OC)cc(F)c1F]

A mixture of 3,4-difluoro-5-methoxybenzoic acid (500 mg, 2.66 mmol, 1 equiv), N,O-dimethylhydroxylamine hydrogen chloride (313 mg, 3.20 mmol, 1.2 equiv), HATU (1.52 g, 3.99 mmol, 1.5 equiv) and DIEA (1.72 g, 13.29 mmol, 5 equiv) in DMF (5 mL) was stirred at ambient temperature for 16 h. The reaction mixture was diluted with EtOAc, washed with brine 3 times, dried over Na₂SO₄ and concentrated under vacuum. The residue was purified by flash chromatography on silica gel (0-40% EtOAc in petroleum ether) to afford 3,4-difluoro-N,5-dimethoxy-N-methylbenzamide (300 mg, 49%) as a white solid. MS (ESI, m/z): 232 (M+H)⁺.

### Step 2: (5-bromothiophen-2-yl)(3,4-difluoro-5-methoxyphenyl)methanone

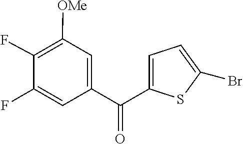 [SMILES: COc1cc(C(=O)c2ccc(Br)s2)cc(F)c1F]

To a stirred solution of 2,5-dibromothiophene (629 mg, 2.59 mmol, 2.00 equiv) in THF (15 mL) at −78° C. was added dropwise a solution of n-BuLi (2.5 M in hexane, 1.70 mL, 2.595 mmol, 2.00 equiv) over 5 mins. The resulting mixture was stirred at this temperature for another 1 h and to this was added 3,4-difluoro-N,5-dimethoxy-N-methylbenzamide (300 mg, 1.298 mmol, 1.00 equiv). The resulting mixture was stirred for another 2 h and then quenched with NH₄Cl solution and extracted with EtOAc 3 times. The combined organic layers were dried over Na₂SO₄ and concentrated under vacuum. The residue was purified by flash chromatography on silica gel (0-400% EtOAc in petroleum ether) to afford (5-bromothiophen-2-yl)(3,4-difluoro-5-methoxyphenyl)methanone (290 mg, 67%) as a pink solid. MS (ESI, m/z): 333 (M+H)⁺.

### Step 3: (S-(2,4-difluoro-3-methoxyphenyl)thiophen-2-yl)(3,4-difluoro-5-methoxyphenyl)methanone

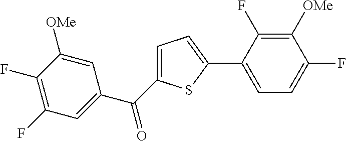 [SMILES: COc1cc(C(=O)c2ccc(-c3ccc(F)c(OC)c3F)s2)cc(F)c1F]

A mixture of (5-bromothiophen-2-yl) (3,4-difluoro-5-methoxyphenyl) methanone (100 mg, 0.300 mmol, 1 equiv), 2,4-difluoro-3-methoxyphenylboronic acid (67.70 mg, 0.360 mmol, 1.2 equiv), t-Bu₃P·HBF₄ (17.42 mg, 0.060 mmol, 0.2 equiv), Pd₂(dba)₃ (27.49 mg, 0.030 mmol, 0.1 equiv) and KF (87.19 mg, 1.501 mmol, 5 equiv) in THF (6 mL) was stirred at 60° C. for 16 h under N2 atmosphere. The reaction mixture was diluted with EtOAc, washed with brine 3 times, dried over Na₂SO₄ and concentrated under vacuum. The residue was purified by flash chromatography on silica gel (0-40% EtOAc in petroleum ether) to afford (5-(2,4-difluoro-3-methoxyphenyl)thiophen-2-yl)(3,4-difluoro-5-methoxyphenyl)methanone (73 mg, 61%) as a yellow solid. MS (ESI, m/z): 397 (M+H)⁺.

### Step 4: (S-(2,4-difluoro-3-hydroxyphenyl)thiophen-2-yl)(3,4-difluoro-5-hydroxyphenyl) methanone (Compound 30)

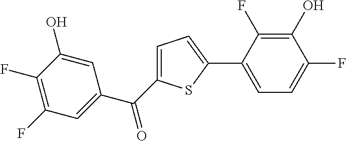 [SMILES: O=C(c1cc(O)c(F)c(F)c1)c1ccc(-c2ccc(F)c(O)c2F)s1]

To a stirred mixture of (5-(2,4-difluoro-3-methoxyphenyl)thiophen-2-yl)(3,4-difluoro-5-methoxyphenyl)methanone (73 mg, 0.184 mmol, 1.00 equiv) in DCM (5 mL) at −78° C. was added dropwise a solution of BBr₃ (1 M in DCM, 1.00 mL, 0.209 mmol, 3.00 equiv) over 5 mins. The resulting solution was allowed to warm up to ambient temperature and stirred for another 2 h. The reaction mixture was quenched with MeOH at 0° C. and then concentrated under vacuum. The residue was purified by Prep-HPLC to afford (5-(2,4-difluoro-3-hydroxyphenyl)thiophen-2-yl)(3,4-difluoro-5-hydroxyphenyl)methanone (12.6 mg, 19%) as a yellow solid. ¹H NMR (300 MHz, DMSO-d₆) δ11.06 (brs, 1H), 10.98 (brs, 1H), 7.79 (dd, J=4.2, 1.2 Hz, 1H), 7.66 (d, J=4.2 Hz, 1H), 7.41-7.28 (m, 3H), 7.24-7.12 (m, 1H). MS (ESI, m/z): 369 (M+H)⁺.

### Example 22: (Z)-5-(3,4-difluoro-5-methoxybenzylidene)-3-(3-hydroxybenzyl)thiazolidine-2,4-dione (Compound 7)

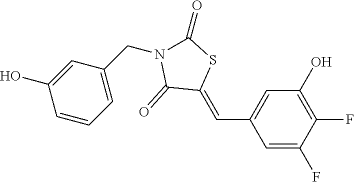 [SMILES: O=C1S/C(=C\c2cc(O)c(F)c(F)c2)C(=O)N1Cc1cccc(O)c1]

The product was synthesized following General Procedure III and 3,4-difluoro-5-methoxybenzaldehyde to afford (Z)-5-(3,4-difluoro-5-methoxybenzylidene)-3-(3-hydroxybenzyl)thiazolidine-2,4-dione (45.1 mg) as a white solid. ¹H NMR (300 MHz, DMSO-d₆) δ 9.94 (brs, 2H), 7.84 (s, 1H), 7.21-6.99 (m, 3H), 6.76-6.63 (m, 3H), 4.73 (s, 2H). MS (ESI, m/z): 364 (M+H)+.

### Example 23: (5-(2,4-difluoro-3-hydroxyphenyl)thiophen-2-yl)(2,4,5-trifluoro-3-hydroxyphenyl) methanone (Compound 31)

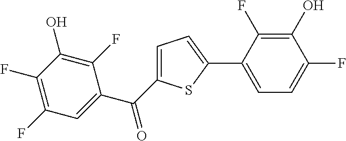 [SMILES: O=C(c1ccc(-c2ccc(F)c(O)c2F)s1)c1cc(F)c(F)c(O)c1F]

The product was synthesized following General Procedure IV starting from 2,4,5-trifluoro-3-methoxybenzoic acid to afford (5-(2,4-difluoro-3-hydroxyphenyl)thiophen-2-yl)(2,4,5-trifluoro-3-hydroxyphenyl)methanone (32.0 mg) as a yellow solid. ¹H NMR (300 MHz, DMSO-d₆) δ11.29 (brs, 1H), 10.65 (brs, 1H), 7.74-7.62 (m, 2H), 7.42-7.29 (m, 1H), 7.28-7.12 (m, 2H). MS (ESI, m/z): 387 (M+H)⁺.

### Example 24: (Z)-3-((5-(4-fluoro-3-hydroxybenzylidene)-2,4-dioxothiazolidin-3-yl)methyl) benzamide (Compound 18)

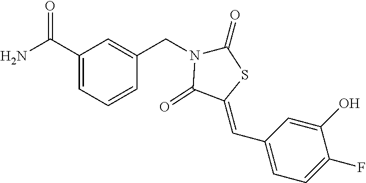 [SMILES: NC(=O)c1cccc(CN2C(=O)S/C(=C\c3ccc(F)c(O)c3)C2=O)c1]

The product was synthesized following General Procedure I starting from Intermediate II and 3-(bromomethyl)benzamide to afford (Z)-3-((5-(4-fluoro-3-hydroxybenzylidene)-2,4-dioxothiazolidin-3-yl)methyl)benzamide (38.7 mg) as a white solid. ¹H NMR (400 MHz, DMSO-d₆) δ 10.40 (s, 1H), 8.01 (brs, 1H), 7.88 (s, 1H), 7.82-7.76 (m, 2H), 7.50-7.39 (m, 2H), 7.38 (s, 1H), 7.32 (dd, J=11.2, 8.4 Hz, 1H), 7.23 (dd, J=8.4, 2.4 Hz, 1H), 7.16-7.09 (m, 1H), 4.88 (s, 2H). MS (ESI, m-z): 373 (M+H)⁺.

### Example 25: (Z)-3(3-hydroxybenzyl)-5-(2,4,5-trifluoro-3-hydroxybenzylidene)thiazolidine-2,4-dione (Compound 3)

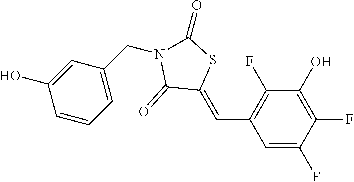 [SMILES: O=C1S/C(=C\c2cc(F)c(F)c(O)c2F)C(=O)N1Cc1cccc(O)c1]

The product was synthesized following General Procedure III and 2,4,5-trifluoro-3-methoxybenzaldehyde to afford (Z)-3-(3-hydroxybenzyl)-5-(2,4,5-trifluoro-3-hydroxybenzylidene)thiazolidine-2,4-dione (46.3 mg) as a white solid. ¹H NMR (300 MHz, DMSO-d₆) δ 11.42 (brs, 1H), 9.49 (s, 1H), 7.84 (s, 1H), 7.18-7.10 (m, 1H), 7.09-6.98 (m, 1H), 6.76-6.64 (m, 2H), 4.75 (s, 2H). MS (ESI, m/z): 382 (M+H)⁺

### Example 26: (Z)-5(2,4-difluoro-3-hydroxybenzylidene)-3-(3-hydroxybenzyl)thiazolidine-2,4-dione (Compound 6)

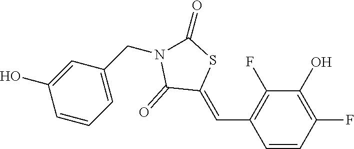 [SMILES: O=C1S/C(=C\c2ccc(F)c(O)c2F)C(=O)N1Cc1cccc(O)c1]

The product was synthesized following General Procedure III and 2,4-difluoro-3-methoxybenzaldehyde to afford (Z)-5-(2,4-difluoro-3-hydroxybenzylidene)-3-(3-hydroxybenzyl)thiazolidine-2,4-dione (10.4 mg) as a white solid. ¹H NMR (400 MHz, DMSO-d₆) δ 10.81 (brs, 1H), 9.50 (brs, 1H), 7.88 (s, 1H), 7.22 (t, J=9.6 Hz, 1H), 7.15 (t, J=7.7 Hz, 1H), 6.99-6.97 (m, 1H), 6.75-6.63 (m, 3H), 4.75 (s, 2H). MS (ESI, m/z): 364 (M+H) ⁺.

### Example 27: (2,4-difluoro-3-hydroxyphenyl)(5-(2,4-difluoro-3-hydroxyphenyl)thiophen-2-yl)methanone (Compound 32)

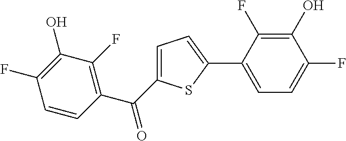 [SMILES: O=C(c1ccc(-c2ccc(F)c(O)c2F)s1)c1ccc(F)c(O)c1F]

The product was synthesized following General Procedure TV starting from 2,4-difluoro-3-methoxybenzoic acid to afford (2,4-difluoro-3-hydroxyphenyl)(5-(2,4-difluoro-3-hydroxyphenyl)thiophen-2-yl)methanone (24.6 mg) as a yellow solid. ¹H NMR (300 MHz, DMSO-d₆) δ 10.64 (d, J=16.8 Hz, 2H), 7.70-7.62 (m, 2H), 7.42-7.28 (m, 1H), 7.27-7.05 (m, 3H). MS (ESI, m/z): 369 (M+H)⁺.

### Example 28: (Z)-5-(2,6-difluoro-3-hydroxybenzylidene)-3-(3-hydroxybenzyl)thiazolidine-2,4-dione (Compound 37)

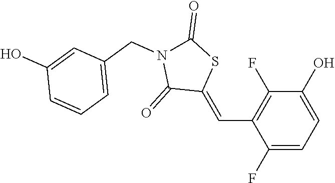 [SMILES: O=C1S/C(=C\c2c(F)ccc(O)c2F)C(=O)N1Cc1cccc(O)c1]

The product was synthesized following General Procedure III and 2,6-difluoro-3-methoxybenzaldehyde to afford (Z)-5-(2,6-difluoro-3-hydroxybenzylidene)-3-(3-hydroxybenzyl)thiazolidine-2,4-dione (52.4 mg) as a white solid. ¹H NMR (400 MHz, DMSO-d₆) δ 10.25 (s, 1H), 9.50 (s, 1H), 7.81 (s, 1H), 7.18-7.02 (m, 3H), 6.76-6.64 (m, 3H), 4.74 (s, 2H). MS (ESI, m/z): 364 (M+H)⁺.

### Example 29: (Z)-5-(4-fluoro-3-hydroxybenzylidene)-3-(3-(trifluoromethyl)benzyl)thiazolidine-2,4-dione (Compound 17)

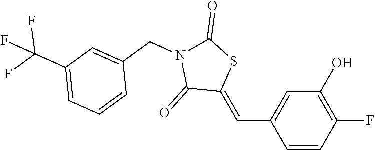 [SMILES: O=C1S/C(=C\c2ccc(F)c(O)c2)C(=O)N1Cc1cccc(C(F)(F)F)c1]

The product was synthesized following General Procedure I starting from Intermediate II and 1-(bromomethyl)-3-(trifluoromethyl)benzene to afford (Z)-5-(4-fluoro-3-hydroxybenzylidene)-3-(3-(trifluoromethyl)benzyl)thiazolidine-2,4-dione (43.6 mg) as a white solid. ¹H NMR (300 MHz, DMSO-d₆) δ 10.38 (s, 1H), 7.88 (s, 1H), 7.74-7.64 (m, 2H), 7.69-7.54 (m, 2H), 7.32 (dd, J=11.1, 8.4 Hz, 1H), 7.23 (dd, J=8.4, 2.4 Hz, 1H), 7.16-7.08 (m, 1H), 4.93 (s, 2H). MS (ESI, m/z): 398 (M+H)˜.

### Example 30: (Z)-3-((5-(4-fluoro-3-hydroxybenzylidene)-2,4-dioxothiazolidin-3-yl)methyl) benzonitrile (Compound 19)

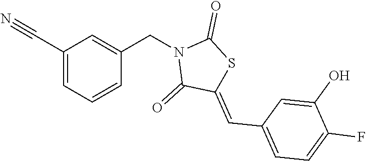 [SMILES: N#Cc1cccc(CN2C(=O)S/C(=C\c3ccc(F)c(O)c3)C2=O)c1]

The product was synthesized following General Procedure I starting from Intermediate U and 3-(bromomethyl)benzonitrile to afford (Z)-3-((5-(4-fluoro-3-hydroxybenzylidene)-2,4-dioxothiazolidin-3-yl)methyl)benzonitrile (106.7 mg) as an off-white solid. ¹H NMR (300 MHz, DMSO-d₆) δ 10.38 (s, 1H), 7.90-7.73 (m, 3H), 7.70-7.62 (m, 1H), 7.57 (t, J=7.8 Hz, 1H), 7.32 (dd, J=11.1, 8.4 Hz, 1H), 7.23 (dd, J=8.4, 2.4 Hz, 1H), 7.17-7.07 (m, 1H), 4.89 (s, 2H). MS (ESI, m % z): 355 (M+H)⁺.

### Example 31: (Z)-5-(4-fluoro-3-hydroxybenzylidene)-3-((tetrahydro-2H-pyran-4-yl)methyl) oxazolidine-2,4-dione (Compound 13)

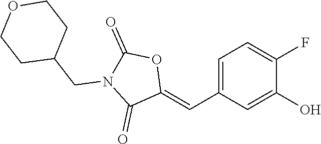 [SMILES: O=C1O/C(=C\c2ccc(F)c(O)c2)C(=O)N1CC1CCOCC1]

### Step 1: 3-(4-fluoro-3-methoxyphenyl)propiolic Acid

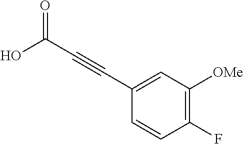 [SMILES: COc1cc(C#CC(=O)O)ccc1F]

A mixture of 4-bromo-1-fluoro-2-methoxybenzene (500.00 mg, 2.440 mmol, 1.00 equiv), propiolic acid (188.70 mg, 2.690 mmol, 1.10 equiv), Pd(PPh₃)₄ (126 mg, 0.121 mmol, 0.05 equiv) and DBU (742 mg, 4.880 mmol, 2.00 equiv) in DMSO (10.00 mL) was stirred at 35° C. for 16 h under N2 atmosphere in accordance with the procedure of the European Journal of Organic Chemistry (Eur. J. Org. 2013, 1973, which is incorporated by reference in its entirety). The reaction mixture was diluted with DCM, washed with brine 3 times, dried over Na₂SO₄ and concentrated under vacuum. The residue was purified by reverse phase flash chromatography on C18 gel (0-60% acetonitrile in water) to afford 3-(4-fluoro-3-methoxyphenyl)propiolic acid (158 mg, 33%) as colorless oil. MS (ESI, m/z): 195 (M+H)⁺.

### Step 2: 3-(4-fluoro-3-methoxyphenyl)-N-((tetrahydro-2H-pyran-4-yl)methyl)propiolamide

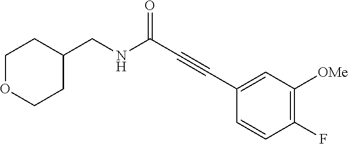 [SMILES: COc1cc(C#CC(=O)NCC2CCOCC2)ccc1F]

To a stirred mixture of 3-(4-fluoro-3-methoxyphenyl)propiolic acid (200.00 mg, 1.03 mmol, 1.00 equiv), (tetrahydro-2H-pyran-4-yl)methanamine (142 mg, 1.23 mmol, 1.20 equiv) and DIEA (400.0 mg, 3.09 mmol, 3.00 equiv) in DMF (5.00 mL) at ambient temperature was added HATU (588 mg, 1.55 mmol, 1.50 equiv). The reaction mixture was stirred at this temperature for 1 h and then diluted with EtOAc. The organic solution was washed with brine 3 times, dried over Na₂SO₄ and concentrated under vacuum. The residue was purified by flash chromatography on silica gel (0-40% EtOAc in petroleum ether) to afford 3-(4-fluoro-3-methoxyphenyl)-N-((tetrahydro-2H-pyran-4-yl)methyl)propiolamide (248 mg, 83%) as brown oil. MS (ESI, m-Z): 292 (M+H)⁺.

### Step 3: (Z)-5-(4-fluoro-3-methoxybenzylidene)-3-((tetrahydro-2H-pyran-4-yl)methyl)oxazolidine-2,4-dione

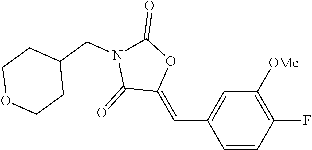 [SMILES: COc1cc(/C=C2\OC(=O)N(CC3CCOCC3)C2=O)ccc1F]

A mixture of 3-(4-fluoro-3-methoxyphenyl)-N-((tetrahydro-2H-pyran-4-yl)methyl)propiolamide (248 mg, 0.85 mmol, 1.00 equiv), K₂CO₃ (353 mg, 2.56 mmol, 3.00 equiv), molecular sieve (4A, 0.5 g) and TBD (20 mg, catalytic amount) in THF (10 mL) was stirred at ambient temperature for 16 h under CO₂ atmosphere. The reaction mixture was diluted with DCM, washed with brine 3 times, dried over Na₂SO₄ and concentrated under vacuum. The residue was purified by flash chromatography on silica gel (0-70% EtOAc in petroleum ether) to afford (Z)-5-(4-fluoro-3-methoxybenzylidene)-3-((tetrahydro-2H-pyran-4-yl)methyl)oxazolidine-2,4-dione (164 mg, 58%) as a white solid. MS (ESI, m/z): 336 (M+H)⁺.

### Step 4: (Z)-5-(4-fluoro-3-hydroxybenzylidene)-3-((tetrahydro-2H-pyran-4-yl)methyl)oxazolidine-2,4-dione (Compound 13)

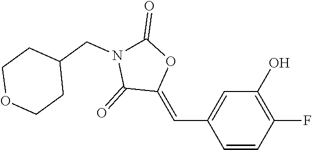 [SMILES: O=C1O/C(=C\c2ccc(F)c(O)c2)C(=O)N1CC1CCOCC1]

To a stirred solution of (Z)-5-(4-fluoro-3-methoxybenzylidene)-3-((tetrahydro-2H-pyran-4-yl)methyl)oxazolidine-2,4-dione (164.00 mg, 0.49 mmol, 1.00 equiv) in DCM (10.00 mL) at −78° C. was added dropwise a solution of BBr₃ (1M in DCM, 2.45 mL, 2.45 mmol, 5.00 equiv) over 5 mins. The resulting solution was allowed to warm up to ambient temperature and stirred for another 16 h. The resulting mixture was then quenched with MeOH at 0° C. and then concentrated under vacuum. The residue was purified by flash chromatography on silica gel (0-80% EtOAc in petroleum ether) to afford (Z)-3-(4-bromo-2-(2-hydroxyethyl)butyl)-5-(4-fluoro-3-hydroxybenzylidene)oxazolidine-2,4-dione (38 mg, 24%) as a white solid. ¹H NMR (300 MHz, DMSO-d₆) δ 10.27 (s, 1H), 7.56-7.47 (m, 1H), 7.32-7.18 (m, 2H), 6.79 (s, 1H), 3.88-3.77 (m, 2H), 3.39 (d, J=7.2 Hz, 2H), 3.30-3.17 (m, 2H), 2.01-1.81 (m, 1H), 1.65-1.54 (m, 2H), 1.30-1.11 (m, 2H). MS (ESI, m/z): 322 (M+H)⁺.

### Example 32: (Z)-5-(4-fluoro-3-hydroxybenzylidene)-3-(3-hydroxybenzyl)thiazolidine-2,4-dione (Compound 11)

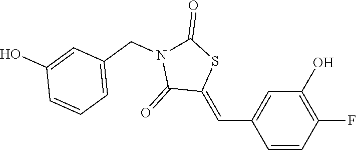 [SMILES: O=C1S/C(=C\c2ccc(F)c(O)c2)C(=O)N1Cc1cccc(O)c1]

The product was synthesized following General Procedure III and 4-fluoro-3-methoxybenzaldehyde to afford (Z)-5-(4-fluoro-3-hydroxybenzylidene)-3-(3-hydroxybenzyl)thiazolidine-2,4-dione (9.9 mg) as a white solid. ¹H NMR (300 MHz, DMSO-d₆) δ 10.40 (s, 1H), 9.48 (s, 1H), 7.88 (s, 1H), 7.38-7.27 (m, 1H), 7.23 (dd, J=8.4, 2.4 Hz, 1H), 7.19-7.07 (m, 2H), 6.76-6.63 (m, 3H), 4.74 (s, 2H). MS (ESI, m/z): 344 (M−H)⁻.

### Example 33: (Z)-5-(4-fluoro-3-hydroxybenzylidene)-3-(3-fluoro-5-hydroxybenzyl)thiazolidine-2,4-dione (Compound 9)

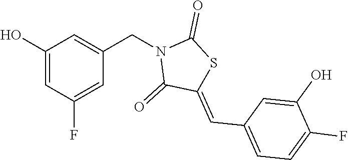 [SMILES: O=C1S/C(=C\c2ccc(F)c(O)c2)C(=O)N1Cc1cc(O)cc(F)c1]

The product was synthesized following General Procedure I starting from Intermediate II and 1-(chloromethyl)-3-fluoro-5-methoxybenzene to afford (Z)-5-(4-fluoro-3-hydroxybenzylidene)-3-(3-fluoro-5-hydroxybenzyl)thiazolidine-2,4-dione (26.9 mg) as an off-white solid. ¹H NMR (300 MHz, DMSO-d₆) δ10.40 (s, 1H), 10.00 (s, 1H), 7.88 (s, 1H), 7.45-7.28 (m, 1H), 7.24 (dd, J=8.4, 2.4 Hz, 1H), 7.18-7.08 (m, 1H), 6.61-6.43 (m, 3H), 4.74 (s, 2H). MS (ESI, m/z): 364 (M+H)⁺.

### Example 34: (Z)-5-(4-fluoro-3-hydroxybenzylidene)-3-(2-fluoro-5-hydroxybenzyl)thiazolidine-2,4-dione (Compound 8)

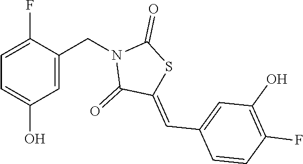 [SMILES: O=C1S/C(=C\c2ccc(F)c(O)c2)C(=O)N1Cc1cc(O)ccc1F]

The product was synthesized following General Procedure I starting from Intermediate II and 2-(bromomethyl)-1-fluoro-4-methoxybenzene to afford (Z)-5-(4-fluoro-3-hydroxybenzylidene)-3-(2-fluoro-5-hydroxybenzyl)thiazolidine-2,4-dione (31.4 mg) as a white solid. ¹H NMR (300 MHz, DMSO-d₆) δ 10.41 (s, 1H), 9.44 (s, 1H), 7.89 (s, 1H), 7.39-6.98 (m, 4H), 6.73-6.58 (m, 2H), 4.80 (s, 2H). MS (ESI, m/z): 364 (M+H)⁺.

### Example 35: 6-fluoro-7-hydroxy-N-(3-hydroxybenzyl)quinoline-2-carboxamide (Compound 24)

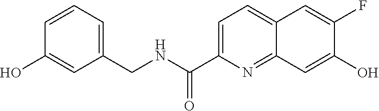 [SMILES: O=C(NCc1cccc(O)c1)c1ccc2cc(F)c(O)cc2n1]

The product was synthesized following General Procedure V starting from Intermediate 4 and 3-(aminomethyl)phenol to afford 6-fluoro-7-hydroxy-N-(3-hydroxybenzyl)quinoline-2-carboxamide (5.2 mg) as a yellow solid. ¹H NMR (300 MHz, DMSO-d₆) δ12.10 (brs, 1H), 11.15 (brs, 1H), 9.39-9.27 (m, 2H), 8.39 (d, J=8.4 Hz, 1H), 7.99 (d, J=8.4 Hz, 1H), 7.85 (d, J=11.4 Hz, 1H), 7.53 (d, J=8.7 Hz, 1H), 7.10 (t, J=8.1 Hz, 1H), 6.76 (d, J=6.3 Hz, 2H), 6.62 (d, J=8.1 Hz, 1H), 4.46 (d, J=6.6 Hz, 2H). MS (EST, m/z): 312 (M+H)⁺.

### Example 36: (Z)-3-(3,4-difluorobenzyl)-5-(4-fluoro-3-hydroxybenzylidene)thiazolidine-2,4-dione (Compound 2)

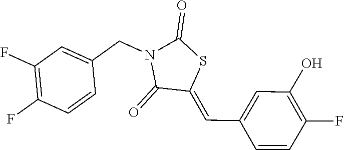 [SMILES: O=C1S/C(=C\c2ccc(F)c(O)c2)C(=O)N1Cc1ccc(F)c(F)c1]

The product was synthesized following General Procedure I starting from Intermediate II and 4-(bromomethyl)-1,2-difluorobenzene to afford (Z)-5-(4-fluoro-3-hydroxybenzylidene)-3-(2-fluoro-5-hydroxybenzyl)thiazolidine-2,4-dione (56.3 mg) as a white solid. ¹H NMR (400 MHz, DMSO-d₆) δ 10.43 (br s, 1H), 7.86 (s, 1H), 7.47-7.36 (m, 2H), 7.31 (dd, J=11.1, 8.4 Hz, 1H), 7.22 (dd, J=8.4, 2.4 Hz, 1H), 7.19-7.08 (m, 2H), 4.82 (s, 2H). MS (ESI, m/z): 364 (M−H)⁻.

### Example 37: N-(3-((2,4-dioxothiazolidin-3-yl)methyl)phenyl)methanesulfonamide (Compound 20)

### Step 1: 3-(3-nitrobenzyl)thiazolidine-2,4-dione

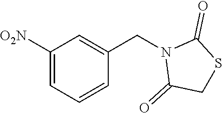 [SMILES: O=C1CSC(=O)N1Cc1cccc([N+](=O)[O-])c1]

A mixture of 1-(bromomethyl)-3-nitrobenzene (200 mg, 0.93 mmol, 1.00 equiv), K₂CO₃ (384 mg, 2.78 mmol, 3.00 equiv) and thiazolidine-2,4-dione (141 mg, 1.20 mmol, 1.30 equiv) in DMF (5.00 mL) was stirred at ambient temperature for 16 h. The resulting mixture was then diluted with EtOAc, washed with brine 3 times, dried over Na₂SO₄ and concentrated under vacuum. The residue was purified by flash chromatography on silica gel (0-50% EtOAc in petroleum ether) to afford 3-(3-nitrobenzyl)thiazolidine-2,4-dione (200 mg, 86%) as a white solid. MS (ESI, m/z): 253 (M+H)⁺.

### Step 2: 3-(3-aminobenzyl)thiazolidine-2,4-dione

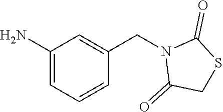 [SMILES: Nc1cccc(CN2C(=O)CSC2=O)c1]

A mixture of 3-(3-nitrobenzyl)thiazolidine-2,4-dione (180 mg, 0.71 mmol, 1.00 equiv), Fe powder (199 mg, 3.57 mmol, 5.00 equiv) and NH₄Cl (115 mg, 2.14 mmol, 3.00 equiv) in EtOH/H₂O (10.00 mL, 10/1 v/v) was stirred at 90° C. for 2 h and then diluted with water and extracted with EtOAc 3 times. The combined organic layers were dried over Na₂SO₄ and concentrated under vacuum. The residue was purified by flash chromatography on silica gel (0-70% EtOAc in petroleum ether) to afford 3-(3-aminobenzyl)thiazolidine-2,4-dione (130 mg, 82%) as a yellow solid. MS (ESI, m/z): 223 (M+H)⁺.

### Step 3: N-(3-((2,4-dioxothiazolidin-3-yl)methyl)phenyl)methanesulfonamide

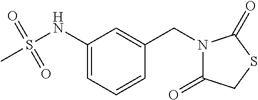 [SMILES: CS(=O)(=O)Nc1cccc(CN2C(=O)CSC2=O)c1]

To a stirred solution of 3-(3-aminobenzyl)thiazolidine-2,4-dione (130.00 mg, 0.59 mmol, 1.00 equiv), and TEA (591.86 mg, 5.85 mmol, 10.00 equiv) in DCM (5 mL) at 0° C. was added dropwise a solution of MsCl (97 mg, 0.644 mmol, 1.1 equiv) in DCM (1.00 mL) over 5 min. The resulting mixture was stirred at this temperature for another 1 h and then quenched with water and extracted with DCM 3 times. The combined organic layers were dried over Na₂SO₄ and concentrated under vacuum. The residue was purified by flash chromatography on silica gel (0-60% EtOAc in petroleum ether) to afford N-(3-((2,4-dioxothiazolidin-3-yl)methyl)phenyl)methanesulfonamide (240 mg, 80%) as a yellow solid. MS (ESI, m/z): 301 (M+H)⁺.

### Step 4: N-(3-((2,4-dioxothiazolidin-3-yl)methyl)phenyl)methanesulfonamide (Compound 20)

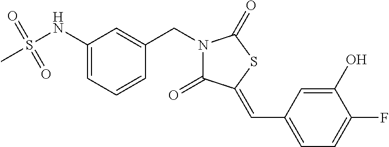 [SMILES: CS(=O)(=O)Nc1cccc(CN2C(=O)S/C(=C\c3ccc(F)c(O)c3)C2=O)c1]

A mixture of N-(3-((2,4-dioxothiazolidin-3-yl)methyl)phenyl)methanesulfonamide (140.00 mg, 0.17 mmol, 1.00 equiv), piperidine (7.9 mg, 0.093 mmol, 0.2 equiv), AcOH (5.6 mg, 0.093 mmol, 0.20 equiv) and 4-fluoro-3-hydroxybenzaldehyde (85 mg, 0.61 mmol, 1.30 equiv) in toluene (50.00 mL) was refluxed for 16 h and then concentrated under vacuum. The residue was purified by flash chromatography on silica gel (0-75% EtOAc in petroleum ether) to afford N-(3-((2,4-dioxothiazolidin-3-yl)methyl)phenyl)methanesulfonamide (11.8 mg, 6%) as a white solid. ¹H NMR (300 MHz, DMSO-d6) δ10.37 (brs, 1H), 9.78 (brs, 1H), 7.88 (s, 1H), 7.38-7.27 (m, 2H), 7.26-7.19 (m, 1H), 7.16-7.14 (m, 3H), 7.03 (d, J=7.8 Hz, 1H), 4.81 (s, 2H), 2.98 (s, 3H). MS (ESI, m/z). 423 (M+H)⁺.

### Example 38: (Z)-5-(4-fluoro-3-hydroxybenzylidene)-3-(3-(trifluoromethoxy) benzyl) thiazolidine-2,4-dione (Compound 16)

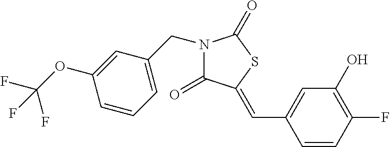 [SMILES: O=C1S/C(=C\c2ccc(F)c(O)c2)C(=O)N1Cc1cccc(OC(F)(F)F)c1]

The product was synthesized following General Procedure I starting from Intermediate U and 1-(bromomethyl)-3-(trifluoromethoxy)benzene to afford (Z)-5-(4-fluoro-3-hydroxybenzylidene)-3-(2-fluoro-5-hydroxybenzyl)thiazolidine-2,4-dione (72.4 mg) as a white solid. ¹H NMR (300 MHz, DMSO-d₆) δ 10.40 (s, 1H), 7.88 (s, 1H), 7.50 (t, J=8.1 Hz, 1H), 7.39-7.27 (m, 4H), 7.23 (dd, J=8.4, 2.4 Hz, 1H), 7.17-7.08 (m, 1H), 4.88 (s, 2H). MS (ESI, m/z): 414 (M+H)⁺.

### Example 39: N-(3-cyanobenzyl)-6-fluoro-7-hydroxyquinoline-2-carboxamide (Compound 27)

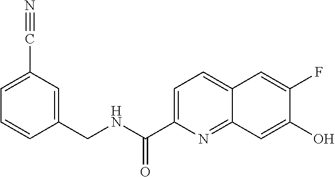 [SMILES: N#Cc1cccc(CNC(=O)c2ccc3cc(F)c(O)cc3n2)c1]

The product was synthesized following General Procedure V starting from 3-(aminomethyl)benzonitrile to afford N-(3-cyanobenzyl)-6-fluoro-7-hydroxyquinoline-2-carboxamide (21 mg) as an off-white solid. ¹H NMR (300 MHz, DMSO-d₆) δ 9.50 (t, J=6.0 Hz, 1H), 8.32 (d, J=8.4 Hz, 1H), 7.90 (d, J=8.4 Hz, 1H), 7.82-7.65 (m, 4H), 7.54 (t, J=7.8 Hz, 1H), 7.46 (d, J=8.7 Hz, 1H), 4.57 (d, J=6.6 Hz, 2H). MS (ESI, m/z): 322 (M+H)+.

### Example 40: N-(3-(1H-tetrazol-5-yl)benzyl)-6-fluoro-7-hydroxyquinoline-2-carboxamide (Compound 25)

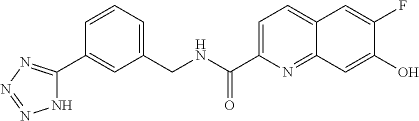 [SMILES: O=C(NCc1cccc(-c2nnn[nH]2)c1)c1ccc2cc(F)c(O)cc2n1]

The product was synthesized following General Procedure V starting from (3-(1H-tetrazol-5-yl)phenyl)methanamine to afford N-(3-(1H-tetrazol-5-yl)benzyl)-6-fluoro-7-hydroxyquinoline-2-carboxamide (8 mg) as a yellow solid. ¹H NMR (300 MHz, DMSO-d₆) δ 9.55 (t, J=6.4 Hz, 1H), 8.41 (d, J=8.4 Hz, 1H), 8.08-7.96 (m, 2H), 7.96-7.81 (m, 2H), 7.61-7.49 (m, 3H), 4.684.59 (m, 2H). MS (ESI, m/z): 365 (M+H)⁺.

### Example 41: (Z)-5-(4-fluoro-3-hydroxybenzylidene)-3-(3-hydroxybenzyl)oxazolidine-2,4-dione (Compound 10)

### Step 1: 3-(4-fluoro-3-methoxyphenyl)propiolic Acid

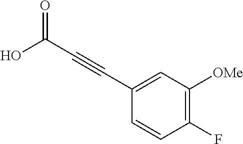 [SMILES: COc1cc(C#CC(=O)O)ccc1F]

A mixture of 4-bromo-1-fluoro-2-methoxybenzene (500.00 mg, 2.440 mmol, 1.00 equiv), propiolic acid (188.70 mg, 2.690 mmol, 1.10 equiv), Pd(PPh₃)₄(126 mg, 0.121 mmol, 0.05 equiv) and DBU (742 mg, 4.880 mmol, 2.00 equiv) in DMSO (10.00 mL) was stirred at 35° C. for 16 h under N2 atmosphere. The reaction mixture was diluted with DCM, washed with brine 3 times, dried over Na₂SO₄ and concentrated under vacuum. The residue was purified by reverse phase flash chromatography on C18 gel (0-60% acetonitrile in water) to afford 3-(4-fluoro-3-methoxyphenyl)propiolic acid (158 mg, 33%) as colorless oil. MS (ESI, m/z): 195 (M+H)-.

### Step 2: 3-(4-fluoro-3-methoxyphenyl)-N-(3-methoxybenzyl)propiolamide

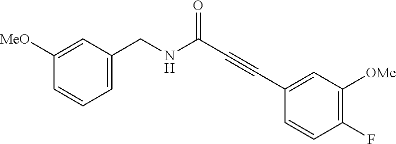 [SMILES: COc1cccc(CNC(=O)C#Cc2ccc(F)c(OC)c2)c1]

To a stirred mixture of 3-(4-fluoro-3-methoxyphenyl)propiolic acid (250.00 mg, 1.280 mmol, 1.00 equiv), (3-methoxyphenyl)methanamine (211.00 mg, 1.540 mmol, 1.20 equiv) and DIEA (498.70 mg, 3.860 mmol, 3.00 equiv) in DMF (5.00 mL) at ambient temperature was added HATU (734.50 mg, 1.930 mmol, 1.50 equiv). The reaction mixture was stirred at this temperature for 1 h and then diluted with EtOAc. The organic solution was washed with brine 3 times, dried over Na₂SO₄ and concentrated under vacuum. The residue was purified by flash chromatography on silica gel (0-40% EtOAc in petroleum ether) to afford 3-(4-fluoro-3-methoxyphenyl)-N-(3-methoxybenzyl)propiolamide (182 mg, 45%) as brown oil. MS (ESI, m/z): 314 (M+H)⁺.

### Step 3: (Z)-5-(4-fluoro-3-methoxybenzylidene)-3-(3-methoxybenzyl)oxazolidine-2,4-dione

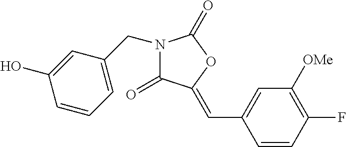 [SMILES: COc1cc(/C=C2\OC(=O)N(Cc3cccc(O)c3)C2=O)ccc1F]

A mixture of 3-(4-fluoro-3-methoxyphenyl)-N-(3-methoxybenzyl)propiolamide (182 mg, 0.580 mmol, 1.00 equiv), K₂CO₃ (241.20 mg, 1.750 mmol, 3.00 equiv), molecular sieve (4A, 0.5 g) and TBD (16 mg, catalytical amount) in THF (10 mL) was stirred at ambient temperature for 16 h under CO₂ atmosphere. The reaction mixture was diluted with DCM, washed with brine 3 times, dried over Na₂SO₄ and concentrated under vacuum. The residue was purified by flash chromatography on silica gel (0-70% EtOAc in petroleum ether) to afford (Z)-5-(4-fluoro-3-methoxybenzylidene)-3-(3-methoxybenzyl)oxazolidine-2,4-dione (130 mg, 63%) as a white solid. MS (EST, m/z): 358 (M+H)⁺.

### Step 4: (Z)-5-(4-fluoro-3-hydroxybenzylidene)-3-(3-hydroxybenzyl)oxazolidine-2,4-dione (Compound 10)

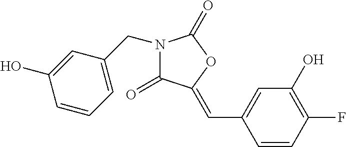 [SMILES: O=C1O/C(=C\c2ccc(F)c(O)c2)C(=O)N1Cc1cccc(O)c1]

To a stirred solution of (Z)-5-(4-fluoro-3-methoxybenzylidene)-3-(3-methoxybenzyl)oxazolidine-2,4-dione (120.00 mg, 0.336 mmol, 1.00 equiv) in DCM (10.00 mL) at −78° C. was added dropwise a solution of BBr₃ (1M in DCM, 1.68 mL, 1.68 mmol, 5.00 equiv) over 5 mins. The resulting solution was allowed to warm up to ambient temperature and stirred for another 16 h. The resulting mixture was then quenched with MeOH at 0° C. and then concentrated under vacuum. The residue was purified by flash chromatography on silica gel (0 ˜80% EtOAc in petroleum ether) to afford (Z)-5-(4-fluoro-3-hydroxybenzylidene)-3-(3-hydroxybenzyl)oxazolidine-2,4-dione (53.2 mg) as a white solid. ¹H NMR (300 MHz, DMSO-d₆) δ 10.25 (s, 1H), 9.48 (s, 1H), 7.57-7.47 (m, 1H), 7.33-7.20 (m, 2H), 7.15 (t, J=7.8 Hz, 1H), 6.86 (s, 1H), 6.81-6.64 (m, 3H), 4.62 (s, 2H). MS (ESI, m/z): 330 (M+H)⁺

### Example 42: 7-fluoro-6-hydroxy-N-(3-hydroxybenzyl)isoquinoline-3-carboxamide (Compound 26)

### Step 1: methyl 2-(((benzyloxy)carbonyl)amino)-3-(4-fluoro-3-methoxyphenyl)acrylate

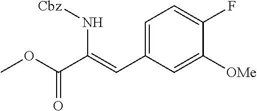 [SMILES: COC(=O)/C(=C/c1ccc(F)c(OC)c1)NC(=O)OCc1ccccc1]

A mixture of 4-fluoro-3-methoxybenzaldehyde (4.70 g, 30.49 mmol, 1.00 equiv), DBU (9.52 mL, 63.72 mmol, 2.1 equiv) and methyl 2-(((benzyloxy)carbonyl)amino)-2-(dimethoxyphosphoryl)acetate (10.10 g, 30.49 mmol, 1.00 equiv) in DCM (200.00 mL) was stirred at ambient temperature for 16 h and then diluted with water and extracted with EtOAc 3 times. The organic layers were combined, dried over Na₂SO₄ and concentrated under vacuum. The residue was purified by flash chromatography on silica gel (0-50% EtOAc in petroleum ether) to afford methyl 2-(((benzyloxy)carbonyl)amino)-3-(4-fluoro-3-methoxyphenyl)acrylate (6.5 g, 59%) as a white solid. MS (ESI, m/z): 360 (M+H)+.

### Step 2: Methyl 2-amino-3-(4-fluoro-3-methoxyphenyl)propanoate

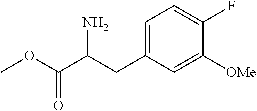 [SMILES: COC(=O)C(N)Cc1ccc(F)c(OC)c1]

A mixture of methyl 2-(((benzyloxy)carbonyl)amino)-3-(4-fluoro-3-methoxyphenyl)acrylate (6.40 g, 17.81 mmol, 1.00 equiv) and Pd/C (wet, 10%/o, 0.64 g, 10% w/w) in EtOAc (10.00 mL, 102.15 mmol, 5.74 equiv) was stirred at ambient temperature for 16 h under H₂ atmosphere and then filtered. The filtrate was concentrated under vacuum to afford methyl 2-amino-3-(4-fluoro-3-methoxyphenyl)propanoate (2.92 g, 72%) as a brown oil, which was used for the next step without further purification. MS (ESI, m/z): 228 (M+H)⁺.

### Step 3: methyl 7-fluoro-6-methoxy-1,2,3,4-tetrahydroisoquinoline-3-carboxylate

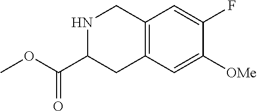 [SMILES: COC(=O)C1Cc2cc(OC)c(F)cc2CN1]

A mixture of methyl 2-amino-3-(4-fluoro-3-methoxyphenyl)propanoate (2.82 g, 12.41 mmol, equiv), TFA (2.83 g, 24.82 mmol, 2.00 equiv) and HCHO (35% solution, 4.26 g, 49.62 mmol, 4.00 equiv) in MeOH (50.00 mL) was stirred at 40° C. for 16 h and then concentrated under vacuum. The residue was purified by flash chromatography on silica gel (0-40%, EtOAc in petroleum ether) to afford methyl 7-fluoro-6-methoxy-1,2,3,4-tetrahydroisoquinoline-3-carboxylate (1.87 g, 63%) as a yellow solid. MS (ESI, m/z): 240 (M+H)⁺.

### Step 4: methyl 7-fluoro-6-methoxyisoquinoline-3-carboxylate

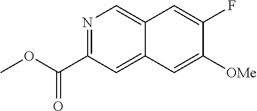 [SMILES: COC(=O)c1cc2cc(OC)c(F)cc2cn1]

A mixture of methyl 7-fluoro-6-methoxy-1,2,3,4-tetrahydroisoquinoline-3-carboxylate (1.81 g, 7.57 mmol, 1.00 equiv) and SeO₂ (1.68 g, 15.14 mmol, 2.00 equiv) in dioxane (18 mL) and pyridine (2 mL) was stirred at 100° C. for 2 h and then concentrated under vacuum. The residue was purified by flash chromatography on silica gel (0-50% EtOAc in petroleum ether) to afford methyl 7-fluoro-6-methoxyisoquinoline-3-carboxylate (500 mg, 28%) as a white solid. MS (ESI, m/z): 236 (M+H)⁺.

### Step 5: 7-fluor-6-hydroxyisoquinoline-3-carboxylic Acid

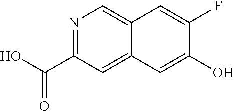 [SMILES: O=C(O)c1cc2cc(O)c(F)cc2cn1]

A mixture of methyl 7-fluoro-6-methoxyisoquinoline-3-carboxylate (200 mg, 0.851 mmol, 1.00 equiv) and LiCl (536 mg, 12.77 mmol, 15.00 equiv) in DMF (10 mL) was stirred at 140° C. for 16 h and then purified directly by reverse phase flash chromatography on C18 gel (0-30% acetonitrile in water) to afford 7-fluoro-6-hydroxyisoquinoline-3-carboxylic acid (70 mg, 40%) as a yellow solid. MS (ESI, m/z): 208 (M+H)⁺.

### Step 6: 7-fluoro-6-hydroxy-N-(3-hydroxybenzyl)isoquinoline-3-carboxamide (Compound 26)

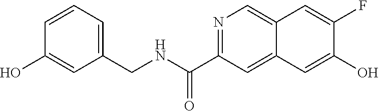 [SMILES: O=C(NCc1cccc(O)c1)c1cc2cc(O)c(F)cc2cn1]

To a stirred solution of 7-fluoro-6-hydroxyisoquinoline-3-carboxylic acid (50.00 mg, 0.242 mmol, 1.00 equiv), 3-(aminomethyl)phenol (30 mg, 0.242 mmol, 1.00 equiv) and DIEA (93 mg, 0.721 mmol, 3.00 equiv) in DMF (2.00 mL) at ambient temperature was added HATU (137 mg, 0.361 mmol, 1.50 equiv). The resulting mixture was stirred at this temperature for 1 h and then directly purified by reverse flash chromatography on C18 silica gel (0-50% acetonitrile in water) to afford 7-fluoro-6-hydroxy-N-(3-hydroxybenzyl)isoquinoline-3-carboxamide (33.1 mg, 44%) as a yellow solid. ¹H NMR (300 MHz, DMSO-d₆) δ 11.13 (brs, 1H), 9.34 (t, J=6.4 Hz, 1H), 8.39 (d, J=8.4 Hz, 1H), 7.99 (d, J=8.4 Hz, 1H), 7.86 (d, J=11.4 Hz, 1H), 7.55 (d, J=8.4 Hz, 1H), 7.12 (t, J=8.1 Hz, 1H), 6.81-6.72 (m, 2H), 6.67-6.55 (m, 1H), 4.514.43 (m, 2H). MS (ESI, m/z): 313 (M+H)⁺.

### Example 43: (Z)-3-((1-(methylsulfonyl)pyrrolidin-3-yl)methyl)-5-(2,4,6-trifluoro-3-hydroxybenzylidene)thiazolidine-2,4-dione (Compound 1)

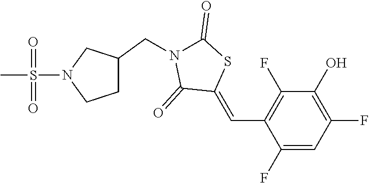 [SMILES: CS(=O)(=O)N1CCC(CN2C(=O)S/C(=C\c3c(F)cc(F)c(O)c3F)C2=O)C1]

The product was synthesized following General Procedure II starting from 3-(bromomethyl)-1-(methylsulfonyl)pyrrolidine to afford (Z)-3-((1-(methylsulfonyl)pyrrolidin-3-yl)methyl)-5-(2,4,6-trifluoro-3-hydroxybenzylidene)thiazolidine-2,4-dione (23.6 mg) as a white solid. ¹H NMR (300 MHz, DMSO-d₆) δ 7.70 (s, 1H), 7.35-7.271 (m, 1H), 3.43-3.24 (m, 2H), 3.23-3.12 (m, 1H), 3.00-2.90 (m, 1H), 2.87 (s, 3H), 2.61-2.51 (m, 1H), 2.07-1.93 (m, 1H), 1.73-1.55 (m, 1H). MS (ESI, m/z): 437 (M+H)⁺

### Example 44: (Z)-5-(4-fluoro-3-hydroxybenzylidene)-3-((tetrahydro-2H-pyran-4-yl)methyl)thiazolidine-2,4-dione (Compound 14)

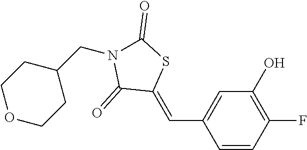 [SMILES: O=C1S/C(=C\c2ccc(F)c(O)c2)C(=O)N1CC1CCOCC1]

The product was synthesized following General Procedure I starting from Intermediate U and 4-(bromomethyl)tetrahydro-2H-pyran to afford (Z)-5-(4-fluoro-3-hydroxybenzylidene)-3-(2-fluoro-5-hydroxybenzyl)thiazolidine-2,4-dione (12 mg) as a white solid. ¹H NMR (300 MHz, DMSO-d₆) δ 10.39 (s, 1H), 7.83 (s, 1H), 7.32 (dd, J=11.1, 8.4 Hz, 1H), 7.22 (dd, J=8.4, 2.4 Hz, 1H), 7.17-7.06 (m, 1H), 3.87-3.76 (m, 2H), 3.54 (d, J=7.2 Hz, 2H), 3.28-3.16 (m, 2H), 2.00-1.84 (m, 1H), 1.56-1.45 (m, 2H), 1.30-1.11 (m, 2H). MS (ESI, m/z): 338 (M+H)⁺.

### Example 45: (Z)-3-((2-oxo-1,2-dihydropyridin-4-yl)methyl)-5-(2,4,6-trifluoro-3-hydroxybenzylidene)thiazolidine-2,4-dione (Compound 36)

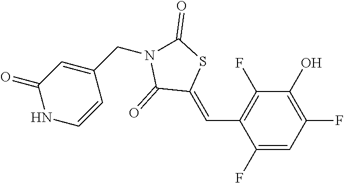 [SMILES: O=C1S/C(=C\c2c(F)cc(F)c(O)c2F)C(=O)N1Cc1cc[nH]c(=O)c1]

A solution of (Z)-3-((2-methoxypyridin-4-yl)methyl)-5-(2,4,6-trifluoro-3-hydroxybenzylidene)thiazolidine-2,4-dione (92 mg, 0.232 mmol, 1.00 equiv) in HBr solution (40%) was stirred at 80° C. for 2 h and then concentrated under vacuum. The residue was purified by reverse phase flash chromatography on C18 gel (5-100% acetonitrile in water) to afford (Z)-3-((2-oxo-1,2-dihydropyridin-4-yl)methyl)-5-(2,4,6-trifluoro-3-hydroxybenzylidene)thiazolidine-2,4-dione (17.2 mg) as a white solid. ¹H NMR (300 MHz, DMSO-d₆) δ 8.37 (s, 1H), 7.76 (s, 1H), 7.34 (d, J=6.6 Hz, 1H), 7.21-7.13 (m, 1H), 6.13 (brs, 1H), 6.07 (dd, J=6.9, 1.8 Hz, 1H), 4.63 (s, 2H). MS (ESI, m/z): 383 (M+H)⁺.

### Example 46: (Z)-3-((6-methoxypyridin-3-yl)methyl)-5-(2,4,6-trifluoro-3-hydroxybenzylidene) thiazolidine-2,4-dione (Compound 37)

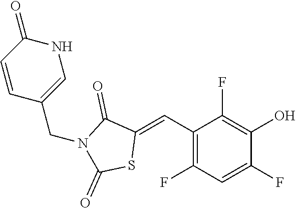 [SMILES: O=C1S/C(=C\c2c(F)cc(F)c(O)c2F)C(=O)N1Cc1ccc(=O)[nH]c1]

A solution of (Z)-3-((6-methoxypyridin-3-yl)methyl)-5-(2,4,6-trifluoro-3-hydroxybenzylidene)thiazolidine-2,4-dione (186.00 mg, 0.469 mmol, 1.00 equiv) in HBr solution (40%) was stirred at 80° C. for 2 h and then concentrated under vacuum. The residue was purified by reverse phase flash chromatography on C18 gel (5-100% acetonitrile in water) to afford (Z)-3-((6-oxo-1,6-dihydropyridin-3-yl)methyl)-5-(2,4,6-trifluoro-3-hydroxybenzylidene)thiazolidine-2,4-dione (25 mg, 14%) as an off-white solid. ¹H NMR (400 MHz, DMSO-d₆) δ 11.58 (s, 1H), 10.57 (s, 1H), 7.72 (s, 1H), 7.43-7.33 (m, 3H), 6.31 (d, J=9.2 Hz, 1H), 4.56 (s, 2H). MS (EST, m/z): 383 (M+H)⁺.

### Example 47: Biochemical Assay for HSD17B13 Inhibition

Compounds of the present disclosure were tested for inhibition of HSD17B13 using the following protocol. Briefly, recombinant human full-length HSD17B13 protein with a 6X-C-terminal His tag was expressed in cell line HEK293E-253/FH and purified using Ni²⁺-affinity purification. Enzymatic activity was determined through the production of NADH using the NAD(P)H-Glo Detection System (Promega). The inhibitory activity of test compounds was determined in reactions performed at 30° C. in assay buffer consisting of 25 mM EPPS (8.0), 125 mM NaCl, 0.03% Triton X-100, 0.03% BSA, with 25 mM steroid substrate (1,3,5(10), 7-estratetraen-3, 17b-diol (Steraloids E0580)) added from a 2.5 mM DMSO stock and 500 mM NAD. The final percentage of DMSO was 4.5%. After a one-hour incubation, a 20% volume of luciferase Glo-reagent was added and light units captured on a Victor V Plate Reader (Perkin Elmer).

Concentration-response curves for inhibitors were normalized to control samples containing enzyme only (negative control) and sample without enzyme (positive control). 50% inhibition (IC50) values were determined using a nonlinear least squares fit of the data to a four-parameter equation using Prism 6.0 (GraphPad Software, San Diego).

TABLE 2
Represents HSD17B13 96 luminescence inhibition assay activity
of compounds of the present disclosure arranged in accordance with the
HSD17B13 96 luminescence inhibition assay IC₅₀ value.

| | |
|---|---|
| Compound | |
| No. | Compound Name |

HSD17B13 96 Luminescence Assay: IC₅₀ (uM) ≤ 1.0

| | |
|---|---|
| 5 | (5Z)-3-[(3- |
| | methanesulfonylphenyl)methyl]- |
| | 5-[(2,4,6-trifluoro-3- |
| | hydroxyphenyl)methylidene]- |
| | 1,3-thiazolidine-2,4-dione |
| 22 | (5Z)-3-[(2-methoxypyridin-4- |
| | yl)methyl]-5-[(2,4,6-trifluoro- |
| | 3-hydroxyphenyl)methylidene]- |
| | 1,3-thiazolidine-2,4-dione |
| 33 | 3-{[(5Z)-5-[(4-fluoro-3- |
| | hydroxyphenyl)methylidene]- |
| | 2,4-dioxo-1,3-thiazolidin-3- |
| | yl]methyl}benzene-1- |
| | sulfonamide |
| 34 | 3-{[(5Z)-2,4-dioxo-5-[(2,4,6- |
| | trifluoro-3- |
| | hydroxyphenyl)methylidene]- |
| | 1,3-thiazolidin-3- |
| | yl]methyl}benzene-1- |
| | sulfonamide |
| 35 | (5Z)-3-[(6-oxo-1,6- |
| | dihydropyridin-3-yl)methyl]-5- |
| | [(2,4,6-trifluoro-3- |
| | hydroxyphenyl)methylidene]- |
| | 1,3-thiazolidine-2,4-dione |
| 38 | (5Z)-5-[(1H-1,2,3-benzotriazol- |
| | 6-yl)methylidene]-3-[(3- |
| | hydroxyphenyl)methyl]-1,3- |
| | thiazolidine-2,4-dione |

HSD17B13 96 Luminescence Assay: 1.0 < IC₅₀ (uM) ≤ 5.0

| | |
|---|---|
| 2 | (5Z)-3-[(3,4- |
| | difluorophenyl)methyl]-5-[(4- |
| | fluoro-3- |
| | hydroxyphenyl)methylidene]- |
| | 1,3-thiazolidine-2,4-dione |
| 3 | (5Z)-3-[(3- |
| | hydroxyphenyl)methyl]-5- |
| | [2,4,5-trifluoro-3- |
| | hydroxyphenyl)methylidene]- |
| | 1,3-thiazolidine-2,4-dione |
| 4 | (5Z)-3-[(3- |
| | hydroxyphenyl)methyl]-5- |
| | [(2,4,6-trifluoro-3- |
| | hydroxyphenyl)methylidene]- |
| | 1,3-thiazolidine-2,4-dione |
| 6 | (5Z)-5-[(2,4-difluoro-3- |
| | hydroxyphenyl)methylidene]-3- |
| | [(3-hydroxyphenyl)methyl]- |
| | 1,3-thiazolidine-2,4-dione |
| 7 | (5Z)-5-[(3,4-difluoro-5- |
| | hydroxyphenyl)methylidene]-3- |
| | [(3-hydroxyphenyl)methyl]- |
| | 1,3-thiazolidine-2,4-dione |
| 8 | (5Z)-5-[(4-fluoro-3- |
| | hydroxyphenyl)methylidene]-3- |
| | [(2-fluoro-5- |
| | hydroxyphenyl)methyl]-1,3- |
| | thiazolidine-2,4-dione |
| 9 | (5Z)-5-[(4-fluoro-3- |
| | hydroxyphenyl)methylidene]-3- |
| | [(3-fluoro-5- |
| | hydroxyphenyl)methyl]-1,3- |
| | thiazolidine-2,4-dione |
| 11 | (5Z)-5-[(4-fluoro-3- |
| | hydroxyphenyl)methylidene]-3- |
| | [(3-hydroxyphenyl)methyl]- |
| | 1,3-thiazolidine-2,4-dione |
| 12 | (5Z)-5-[(4-fluoro-3- |
| | hydroxyphenyl)methylidene]-3-[(3- |
| | methanesulfonylphenyl)methyl]- |
| | 1,3-thiazolidine-2,4-dione |
| 13 | (5Z)-5-[(4-fluoro-3- |
| | hydroxyphenyl)methylidene]-3- |
| | [(oxan-4-yl)methyl]-1,3- |
| | oxazolidine-2,4-dione |
| 15 | (5Z)-5-[(4-fluoro-3- |
| | hydroxyphenyl)methylidene]-3- |
| | {(3-(1H-1,2,3-triazol-4- |
| | yl)phenyl]methyl}-1,3- |
| | thiazolidine-2,4-dione |
| 17 | (5Z)-5-[(4-fluoro-3- |
| | hydroxyphenyl)methylidene]-3-{[3- |
| | (trifluoromethyl)phenyl]methyl}- |
| | 1,3-thiazolidine-2,4-dione |
| 18 | 3-{[(5Z)-5-[(4-fluoro-3- |
| | hydroxyphenyl)methylidene]- |
| | 2,4-dioxo-1,3-thiazolidin-3- |
| | yl]methyl}benzamide |
| 19 | 3-{[(5Z)-5-[(4-fluoro-3- |
| | hydroxyphenyl)methylidene]- |
| | 2,4-dioxo-1,3-thiazolidin-3- |
| | yl]methyl}benzonitrile |
| 23 | (5Z)-3-[(6-methoxypyridin-3- |
| | yl)methyl]-5-[(2,4,6-trifluoro- |
| | 3-hydroxyphenyl)methylidene]- |
| | 1,3-thiazolidine-2,4-dione |
| 24 | 6-fluoro-7-hydroxy-N-[(3- |
| | hydroxyphenyl)methyl]quinoline- |
| | 2-carboxamide |
| 28 | 2,6-difluoro-3-[5-(4-fluoro-3- |
| | hydroxybenzoyl)thiophen-2- |
| | yl]phenol |
| 29 | 3-[5-(2,4-difluoro-3- |
| | hydroxyphenyl)thiophene-2- |
| | carbonyl]-2,4,6-trifluorophenol |
| 30 | 3-[5-(3,4-difluoro-5- |
| | hydroxybenzoyl)thiophen-2- |
| | yl]-2,6-difluorophenol |
| 31 | 3-[5-(2,4-difluoro-3- |
| | hydroxyphenyl)thiophene-2- |
| | carbonyl]-2,5,6-trifluorophenol |
| 32 | 3-[5-(2,4-difluoro-3- |
| | hydroxybenzoyl)thiophen-2- |
| | yl]-2,6-difluorophenol |
| 36 | (5Z)-3-[(2-oxo-1,2- |
| | dihydropyridin-4-yl)methyl]-5- |
| | [(2,4,6-trifluoro-3- |
| | hydroxyphenyl)methylidene]- |
| | 1,3-thiazolidine-2,4-dione |
| 39 | 6,8-difluoro-7-hydroxy-N-[(3- |
| | methoxyphenyl)methyl]quinoline- |
| | 2-carboxamide |

HSD17813 96 Luminescence Assay: 5.0 < IC₅₀ (uM)

| | |
|---|---|
| 1 | (5Z)-3-[(1- |
| | methanesulfonylpyrrolidin-3- |
| | yl)methyl]-5-[(2,4,6-trifluoro- |
| | 3-hydroxyphenyl)methylidene]- |
| | 1,3-thiazolidine-2,4-dione |
| 10 | (5Z)-5-[(4-fluoro-3- |
| | hydroxyphenyl)methylidene]- |
| | 3-[(3-hydroxyphenyl)methyl]- |
| | 1,3-oxazolidine-2,4-dione |
| 14 | (5Z)-5-[(4-fluoro-3- |
| | hydroxyphenyl)methylidene]- |
| | 3-[(oxan-4-yl)methyl]-1,3- |
| | thiazolidine-2,4-dione |
| 16 | (5Z)-5-[(4-fluoro-3- |
| | hydroxyphenyl)methylidene]- |
| | 3-{[3- |
| | (trifluoromethoxy)phenyl]methyl}- |
| | 1,3-thiazolidine-2,4-dione |
| 20 | N-(3-{[(5Z)-5-[(4-fluoro-3- |
| | hydroxyphenyl)methylidene]- |
| | 2,4-dioxo-1,3-thiazolidin-3- |
| | yl]methyl}phenyl)methanesulfonamide |
| 25 | 6-fluoro-7-hydroxy-N-{[3-(1H- |
| | 1,2,3,4-tetrazol-5- |
| | yl)phenyl]methyl}quinoline-2- |
| | carboxamide |
| 26 | 7-fluoro-6-hydroxy-N-[(3- |
| | hydroxyphenyl)methyl]isoquinoline- |
| | 3-carboxamide |
| 27 | N-[(3-cyanophenyl)methyl]-6- |
| | fluoro-7-hydroxyquinoline-2- |
| | carboxamide |
| 37 | (5Z)-5-[(2,6-difluoro-3- |
| | hydroxyphenyl)methylidene]- |
| | 3-[(3-hydroxyphenyl)methyl]- |
| | 1,3-thiazolidine-2,4-dione |

### Example 48: Oral and Subcutaneous Bioavailability of HSD17B13 Inhibitors

To examine oral exposure in mice, the HSD17B13 inhibitors will be tested. Briefly, mice will be orally administered HSD17B13 inhibitors. Clearance and oral bioavailability will be measured.

### Example 49: Inhibition of Inflammation by HSD17B13 Inhibitors

To examine the effect of HSD17B13 inhibitors on inflammatory cytokine expression HepG2 cells were exposed to various concentrations of HSD17B13 inhibitors. Briefly, HepG2 cells, an immortalized human liver cancer cell line, were modified to overexpress HSD17B13. Cells were grown in EMEM (ATCC) with 10% FBS and penicillin/streptomycin. One day prior to transfection 750,000 cells were plated per well in a 6 well plate and cultured overnight. 2 mgs of pCMV6-Entry-HSD17B13 or empty vector (Origene) were transfected using Lipofectamine 3000 (Invitrogen) and the cells were cultured for 24 hours. Cells were split into selection media (1000 mg/ml G418 (Gibco)) and a stable pool was established and verified by qPCR and western blotting using an anti-Flag antibody (Genscript). HepG2 cells overexpressing HSD17B13 or empty vector control, were seeded at 400,000 cells per well in 12 well plates and cultured overnight. Media was replaced with serum-free EMEM and cultured an additional night. A 12 mM fatty acid stock of oleic acid/palmitate at a 2:1 ratio was prepared in 10% fatty acid free BSA (Sigma) by combining 8 mM oleic acid and 4 mM palmitate and rotating overnight at 30° C. The solution was then sonicated for an additional 45 minutes to solubilize.

The serum-starved cells were challenged for 3 hours with 1.2 mM fatty acids with or without 10 μM compound added one hour prior to challenge. The cells were lysed in RLT buffer and RNA purified using a Qiagen RNeasy mini kit. RNA quality and concentration were determined on a Nanodrop. cDNA was synthesized from 1 μg RNA using iScript reverse transcription supermix (Biorad) and rtPCR performed with 1 μl cDNA using SsoAdvanced Universal Probes Supermix (Biorad). Taqman probes (Thermo Fisher) were used to look at effects of fatty acids and HSD17B13 inhibitors on IL-8 and IL-32 gene expression using GAPDH as an endogenous control.

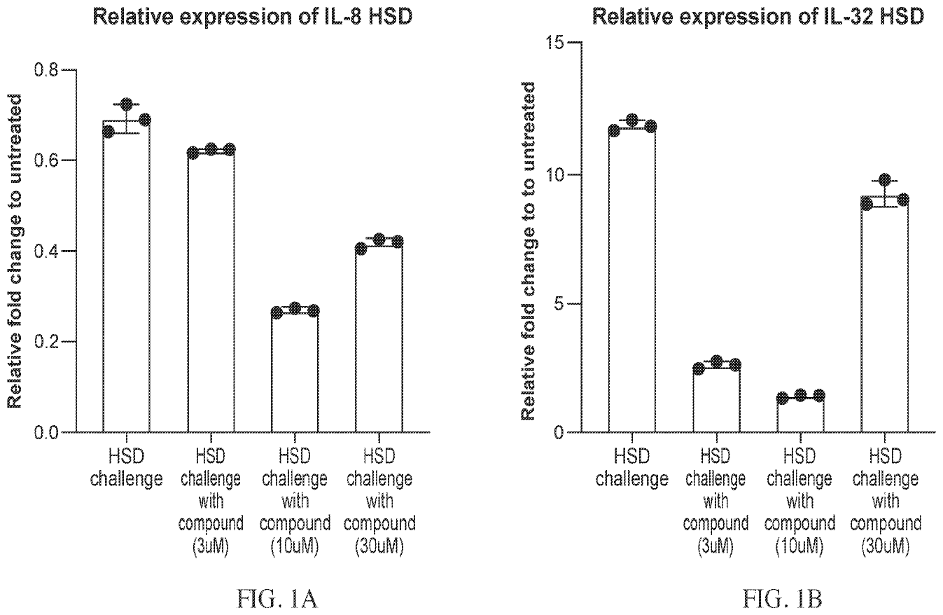

As shown in FIG. 1A, relative expression levels of IL-8 decreased following 3-hour exposure to 3 μM, 10 μM, or 30 μM Compound 5. In addition, relative expression levels of IL-32 decreased following 3-hour exposure to 3 μM, 10 μM, or 30 μM Compound 5 (FIG. 1B). In particular, a 60-70% reduction in IL-32 expression was observed at the 3 μM dose. It is contemplated that some toxicity to the cells occurred at 30 μM. Data is expressed as expression fold change relative to untreated cells.

This data demonstrates that HSD17B13 small molecule inhibitors exhibit functional cellular inhibition and inhibit increased expression of inflammatory cytokines.

### Example 50: Differentiation of iPSCs into Hepatocytes

Hepatocytes were derived from human induced pluripotent stem cells (hiPSCs) using standard methods to determine whether they exhibit characteristics of primary hepatocytes.

Using a first differentiation method (method #1), the iPSC-derived hepatocytes expressed common makers including, albumin, HNF4-alpha, and ASGPR1 at day 23 (data not shown). ASGR1 expression was also analyzed by fluorescence activated cell sorting (FACS) and greater than 85% of cells were ASGR1 positive (data not shown).

Hepatocytes were also derived using the Cellartis iPS Cell to Hepatocyte Differentiation System from Takara (method #2). Induced PSCs were cultured and differentiated into hepatocytes for 28 or 34 days according to the suppliers' recommendations in the Cellartis Hepatocyte Differentiation Kit. Briefly, the cell culture surface (cell culture plates or scaffold) was coated with Hepatocyte Coating (from Cellartis Hepatocyte Differentiation Kit, Cat. No. Y30050) at 37° C. for 1-2 days and subsequently washed with phosphate buffered saline solution (10 mM Na phosphate in 0.9% NaCl, pH 7.4). Definitive endoderm (DE) cells were thawed and seeded in Hepatocyte Thawing and Seeding Medium at an initial density of 2.5×10⁶ cells/scaffold in 24-well plate format (using polystyrene (PS) well plates) in 1 ml of medium. The DE cells were differentiated in Hepatocyte Thawing and Seeding Medium for 2 days at 37° C., before changing to Hepatocyte Progenitor Medium for another 5 days of differentiation to hepatoblasts. The cells were then differentiated further in Hepatocyte Maturation Medium for 4 days to immature hepatocytes and finally matured in Hepatocyte Maintenance Medium for another 17 days 37 days of culture to mature hepatocytes.

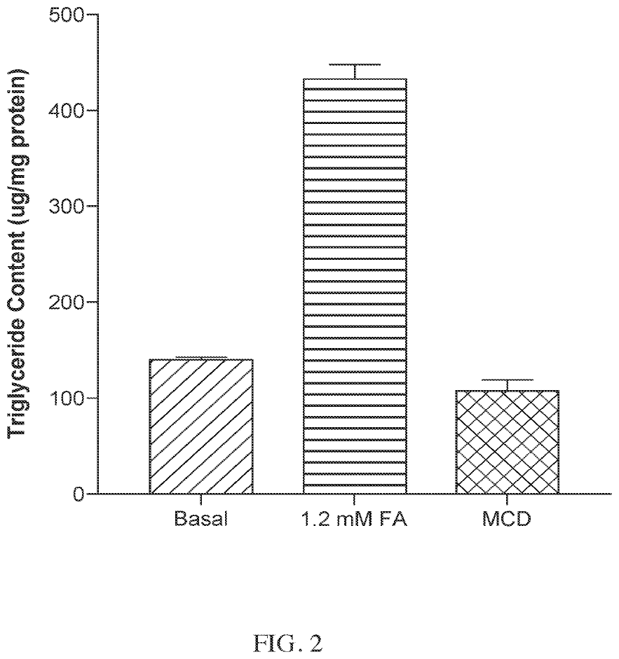

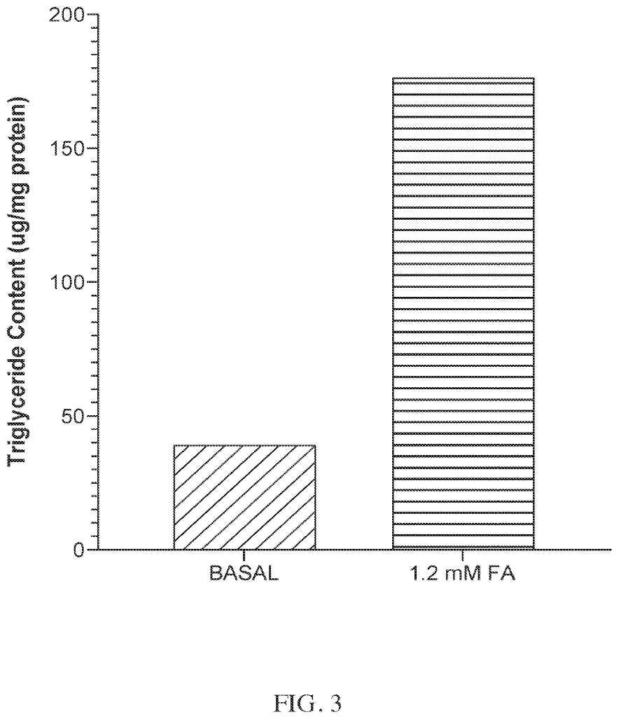

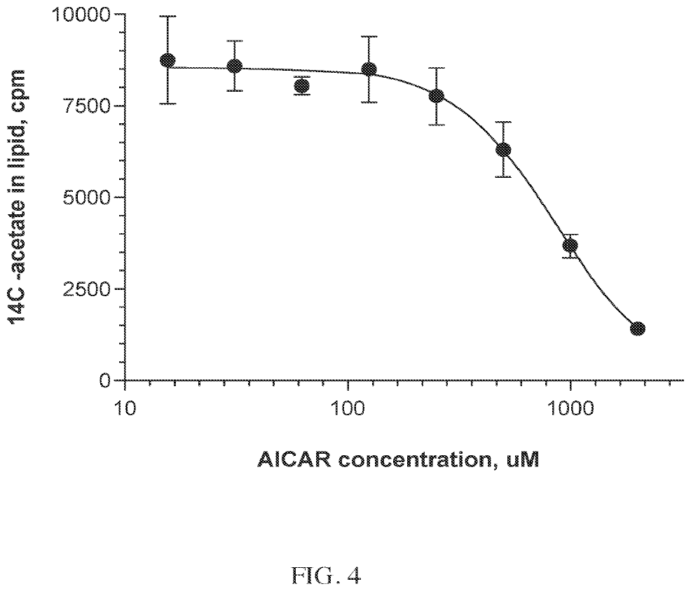

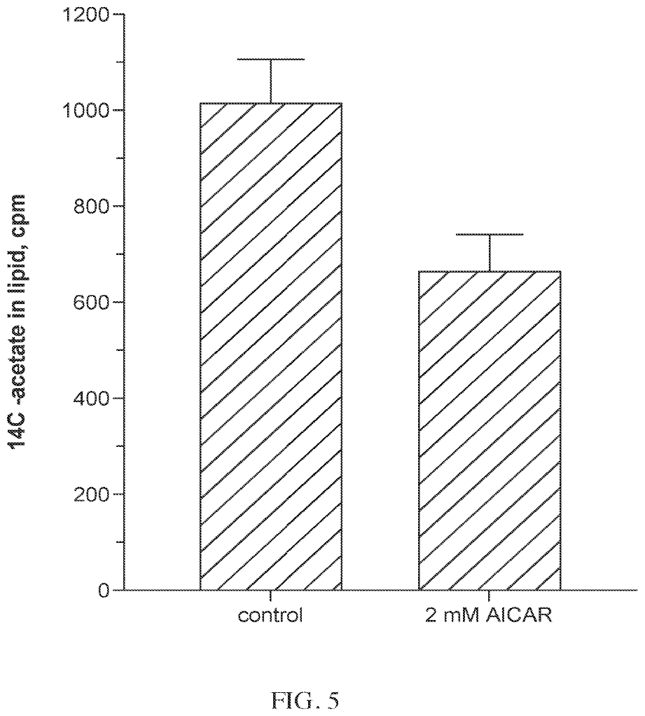

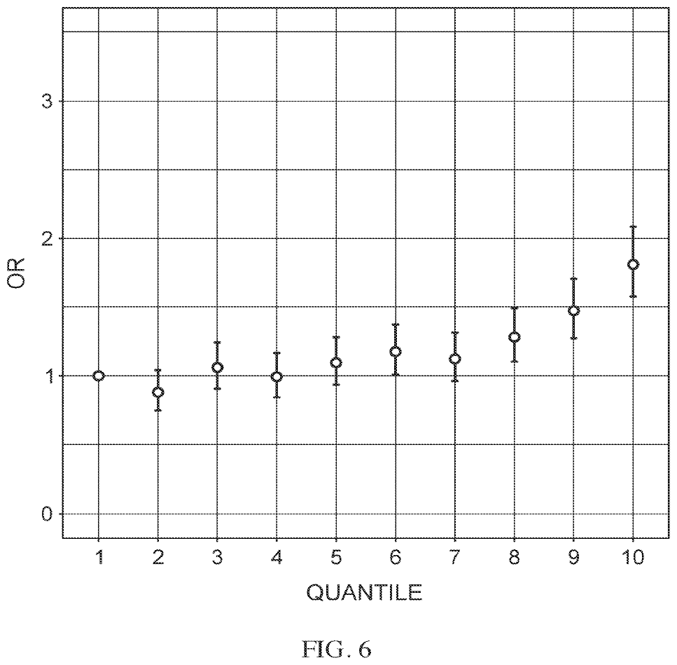

Next, the in vitro derived hepatocytes were tested for fatty acid (“FA”) (2:1 oleic acid/palmitic acid) induced steatosis. Briefly, triglyceride levels were measured in iPSC-derived hepatocytes following 24-hour treatment with 1.2 mM FA following a protocol similar to as described above. As shown in FIG. 2 (method #1) and FIG. 3 (method #2), iPSC-derived hepatocytes from both differentiation protocols produced similar steatosis on treatment with FA stimulation. iPSC-derived hepatocytes also demonstrated de novo lipogenesis (DNL) (FIG. 5 and FIG. 6). Together, these data show that iPSC-derived hepatocytes display cellular markers and functionality characteristic of mature hepatocytes. It is contemplated that exposure to HSD17B13 inhibitors described herein will reduce the levels of HSD17B13 in the of iPSC-derived hepatocytes.

### Example 51: Evaluating HSD17B13 Inhibitors on Preventing or Treating NASH in Mice

C57Bl/6J mice, aged 5-6 weeks (Jackson Labs) will be housed 4 per cage on non-nutritive bedding (Alpha-dri). Mice will be housed in a 12 h day/night cycle and allowed water ad libitum. Mice will be placed on the following diets upon arrival: Control Diet (LabDiet, Rodent Diet 5001, n=24) or choline deficient high fat diet (CDHFD) (L-Amino Acid Diet with 60 kcal % Fat with 0.1% Methionine and No Added Choline, Research Diets, #A06071302, n=48). In Prevention Mode, dosing with vehicle or HSD17B13 inhibitors will commence on Day 0. In Treatment Mode, mice will be fed aforementioned diets ad libitum and dosing commencing on Day 28 (Week 4). Mice will be maintained on specified diets while dosing continued QD throughout the duration of the studies.

CDHFD diet will be changed frequently to avoid rancidity. Body weights will be recorded weekly in either mode. Mice will be sacrificed (n=8) from each group at the following intervals: Week 8, 12 and 16. Mice will be anesthetized via isoflurane and bled via cardiac stick. Plasma will be analyzed for triglyceride levels and circulating markers of NASH including osteopontin, FGF-21 and MCP-1 levels as well as AST activity. Livers will then be excised and snap frozen immediately. Liver tissue will be sent for histopathology (Charles River Labs) and NASH Activity Score. Liver will also be analyzed for triglyceride levels. It is contemplated that NASH activity scores and triglyceride levels will be reduced for mice treated with an HSD17B13 inhibitor as compared to vehicle control.

### Example 52: Determining Polygenic Risk Score for Chronic Fatty Liver Disease and Liver-Related Morbidity and Mortality

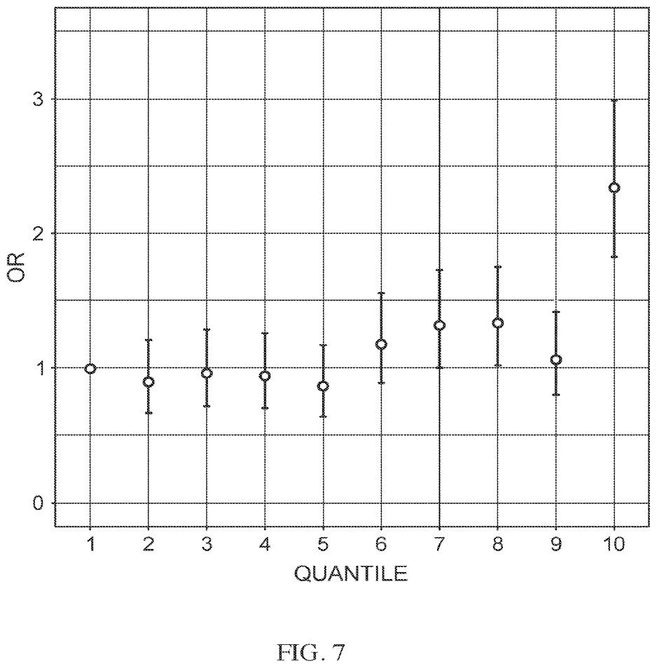

Polygenic risk scores can inform on a person's risk of a certain disease as compared to others with a different genetic makeup. To construct weighted polygenic risk scores for chronic liver disease allele dosages were summed for each of six variant sites that reproducibly associate with chronic fatty liver disease (rs72613567, rs738409, rs58542926, rs641738, rs2642438, rs28929474), weighted by effect estimates from Gastroenterology 2016; 150:1219-1230.e6; PLoS Genet 2020:16: e1008629; and N Engl J Med 2018; 378:1096-1106. Associations of this score with chronic fatty liver and cirrhosis disease were evaluated in the UK Biobank, using imputed individual-level genotype data in up to 3,594 chronic liver disease cases or 1,092 cirrhosis cases, defined according to hospital encounter diagnosis codes and self-reported disease status, and up to 353,996 study subjects without corresponding hospital encounter disease diagnosis codes or self-reported disease. Odds ratios of disease for each decile of polygenic score were modeled using logistic regression adjusted for age, sex, chip identifier, and the first six principal components of ancestry, with the first decile of polygenic score as the reference group. As shown in FIG. 6 and FIG. 7, there is a score dosage-dependent increase in odds of disease, such that the top decile of polygenic score is associated with >2.7× fold higher odds of cirrhosis and nearly 2× higher odds of chronic liver disease when compared to the lowest decile.

Polygenic risk score for liver-related morbidity and mortality will be calculated. Power and accuracy of the polygenic risk score described above will be evaluated for predicting incident liver-related endpoints in the UK Biobank (cirrhosis, decompensated cirrhosis, hepatocellular carcinoma, liver transplant, end stage liver disease, and liver-related mortality, defined by hospital encounter diagnosis and procedure codes) and these observations may be confirmed in an external cohort. De novo genome-wide Cox proportional hazards regression will be used to more precisely estimate weights for an incident outcome polygenic risk score, which will subsequently be constructed using effect estimates for an approximately independent set of variants associated with incident outcomes at P<5e-02.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (I): 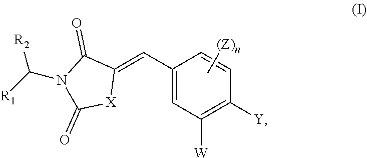
or a pharmaceutically acceptable salt, ester, solvate, amino acid conjugate, isomer, or tautomer thereof, wherein:
X is selected from O and S;
Y is selected from —H, —OH and —F;
W is —OH; or
W and Y, taken together with the atom to which they are each attached, form a 3- to 10-membered heterocycle or heteroaryl, wherein the heterocycle or heteroaryl is optionally substituted with one or more Z;
each Z is independently, at each occurrence, selected from halogen, —OH, —NH₂, —CN, C₁-C₄ alkyl, C₁-C₄ alkoxy, C₂-C₄ alkenyl, and C₂-C₄ alkynyl;
R₁ is independently selected from C₃-C₆ cycloalkyl, tetrahydropyranyl, tetrahydrofuran, pyridinyl, piperidinyl, pyrrolidinyl, pyridonyl, and phenyl, wherein the phenyl is substituted with one or more R₃, and wherein the cycloalkyl, tetrahydropyranyl, tetrahydrofuranyl, pyridinyl, piperidinyl, pyridonyl, or pyrrolidinyl is optionally substituted with one or more R₄;
R₂ is selected from —H or —CH₃;
each R₃ is independently selected from halogen, —OH, C₁-C₆ alkoxy, —CF₃, haloalkoxy, —NO₂, —S(O)₂R₅, —NHSO₂C₁-C₄ alkyl, —NHCOC₁-C₄ alkyl, —CF₃, —COOH, —C(O)NH₂, C(O)NHR₅, S(O)₂NH₂, S(O)₂NHR₅, —CN, —C(O)OR₅, —NH₂, and heteroaryl;
each R₄ is independently selected from oxo, C₁-C₆ alkyl, C₁-C₄ alkoxy, —C(O)R₅, —CF₃, and —S(O)₂R₅;
each R₅ is independently selected from —H, C₁-C₆ alkyl, C₁-C₆ alkoxy, C₂-C₆ alkenyl, C₂-C₆ alkynyl, and —CF₃; and
n is an integer selected from 0, 1, and 2.

2. The compound of claim 1 having the Formula (Ia): 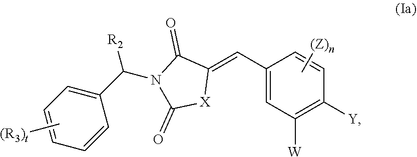
wherein:
R₃ is selected from —OH, —CF₃, —F, —NHSO₂Me, —S(O)₂—C₁-C₄alkyl, C₁-C₄ alkoxy, C(O)NH₂, C(O)NHR₆, S(O)₂NH₂, S(O)₂NHR₅, and heteroaryl;
t is an integer selected from 0, 1, 2, and 3; and
Z is —F.

3. The compound of claim 1 having the Formula (Ib): 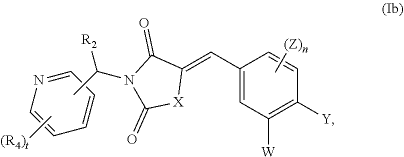
wherein:
R₄ is selected from —OH, —CF₃, —F, —NHSO₂Me, —S(O)₂—C₁-C₄alkyl, C₁-C₄ alkoxy, and heteroaryl;
t is an integer selected from 0, 1, 2, and 3; and
Z is F.

4. The compound of claim 1 having the Formula (Ic): 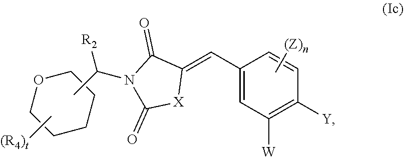
wherein:
R₄ is selected from —OH, —CF₃, —F, —NHSO₂Me, —S(O)₂—C₁-C₄alkyl, C₁-C₄ alkoxy, and heteroaryl;
t is an integer selected from 0, 1, 2, 3, 4, and 5; and
Z is F.

5. The compound of claim 1 having the Formula (Id): 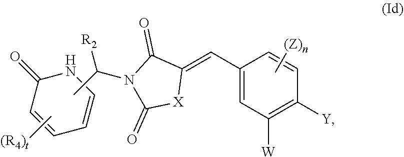
wherein:
R₄ is selected from —OH, —CF₃, —F, —NHSO₂Me, S(O)₂—C₁-C₄alkyl, C₁-C₄ alkoxy, and heteroaryl;
t is an integer selected from 0, 1, 2, and 3; and
Z is F.

6. A compound of Formula II: 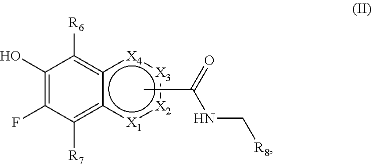
or a pharmaceutically acceptable salt, ester, solvate, amino acid conjugate, isomer, or tautomer thereof, wherein:
X₁ is selected from bond, C(R₁₀), and N;
X₂, X₃, and X₄ are independently selected from C(R₁₀), N, O, and S; provided that at least one of X₂, X₃, and X₄ is C(R₁₀) or N;
R₆ and R₇ are independently selected from —H, halogen, —NR₁₀R₁₁, —C(O)R₁₀, —C(O)NR₁₀R₁₁, —CN, C₁-C₆ alkyl, C₁-C₆ alkoxy, C₂-C₆ alkenyl, C₂-C₆ alkynyl, C₃-C₁₀ cycloalkyl, C₅-C₈ cycloalkenyl, C₅-C₈ spirocycloalkyl, spiroheterocyclyl, heterocyclyl, aryl, and heteroaryl, wherein the alkyl, alkoxy, alkenyl, alkynyl, cycloalkyl, cycloalkenyl, spirocycloalkyl, spiroheterocyclyl, heterocyclyl, aryl, or heteroaryl is optionally substituted with one or more R₉;
R₈ is selected from phenyl and heteroaryl, wherein the phenyl or heteroaryl is optionally substituted with one or more —OH, C₁-C₄ alkoxy, —S(O)₂—C₁-C₃ alkyl, —COOH, —CN, —CONH₂, or heteroaryl containing 1-4 heteroatoms selected from N, O, and S;
R₉, R₁₀, and R₁₁ are independently at each occurrence, selected from —H, halogen, C₁-C₆ alkyl, C₁-C₆ alkoxy, C₂-C₆ alkenyl, C₂-C₆ alkynyl, C₃-C₈ cycloalkyl, heterocyclyl, aryl, and heteroaryl.

7. The compound of claim 6 having the Formula Ha: 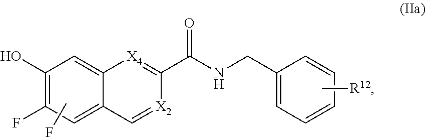
wherein:
R₁₂ is selected from —OH, C₁-C₄ alkoxy, —S(O)₂—C₁-C₄ alkyl, —F, —CN, C₁-C₄alkyl,

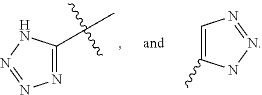

8. A compound of Formula II: 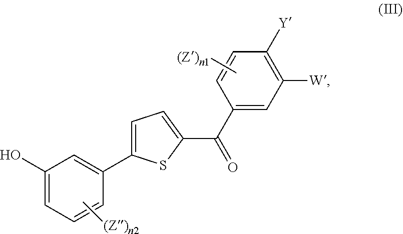
or a pharmaceutically acceptable salt, ester, solvate, amino acid conjugate, isomer, or tautomer thereof, wherein:
W′ is selected from —H, —OH, and —F;
Y′ is selected from —OH and —F;
each Z′ and Z″ are, at each occurrence, independently selected from halogen, —OH, —NH₂, —CN, C₁-C₄ alkyl, C₁-C₄ alkoxy, C₂-C₄ alkenyl, or C₂-C₄ alkynyl;
n1 is an integer selected from 0, 1, 2, or 3; and
n2 is an integer selected from 0, 1, 2, 3, or 4.

9. The compound of claim 1 selected from the group consisting of:

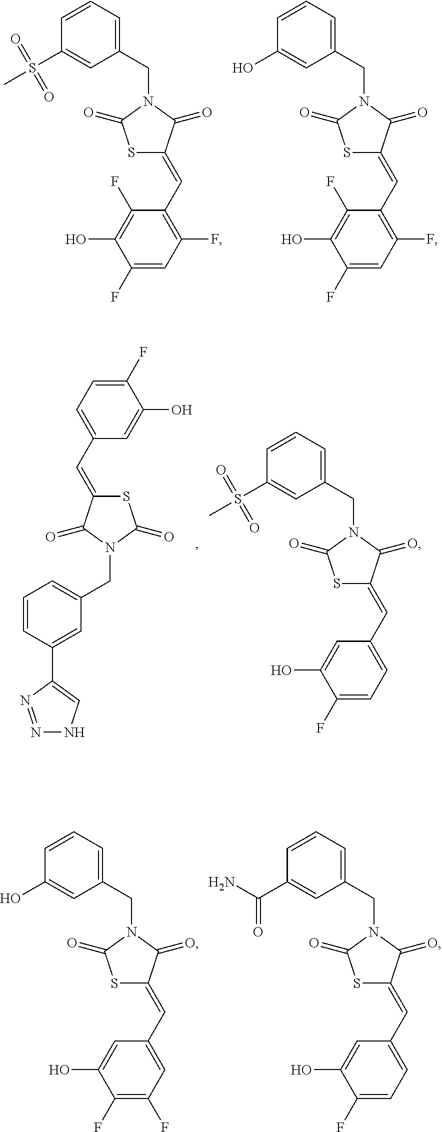

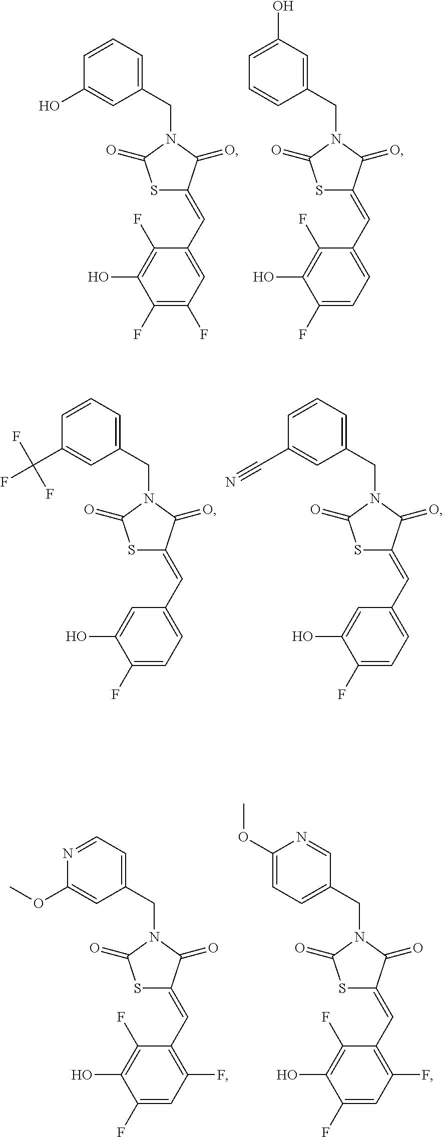

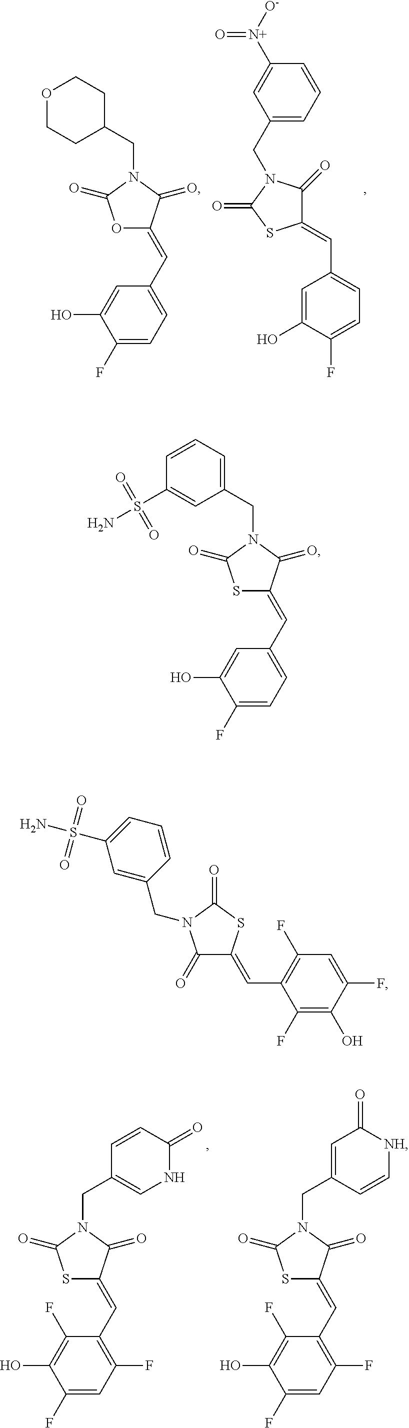

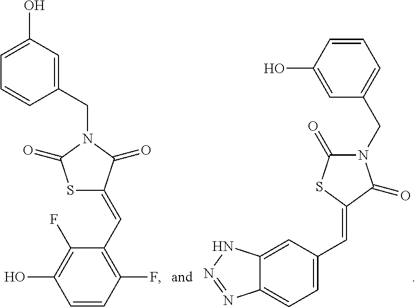 [SMILES: O=C1S/C(=C\c2c(F)ccc(O)c2F)C(=O)N1Cc1cccc(O)c1 ; O=C1S/C(=C\c2ccc3nn[nH]c3c2)C(=O)N1Cc1cccc(O)c1]

10. The compound of claim 6 selected from the group consisting of:

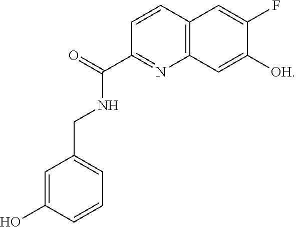 [SMILES: O=C(NCc1cccc(O)c1)c1ccc2cc(F)c(O)cc2n1]

11. The compound of claim 8 selected from the group consisting of:

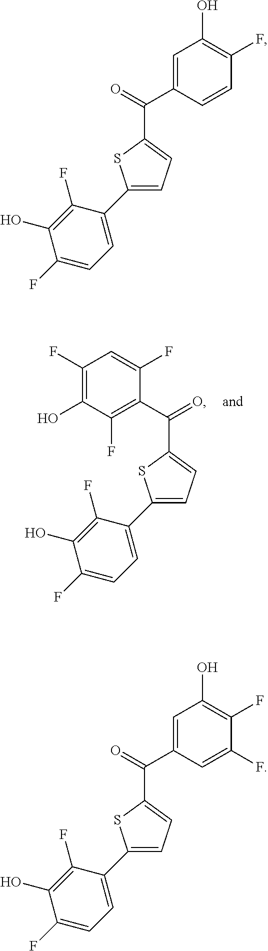 [SMILES: O=C(c1ccc(F)c(O)c1)c1ccc(-c2ccc(F)c(O)c2F)s1 ; O=C(c1ccc(-c2ccc(F)c(O)c2F)s1)c1c(F)cc(F)c(O)c1F ; "and" ; O=C(c1cc(O)c(F)c(F)c1)c1ccc(-c2ccc(F)c(O)c2F)s1]

12. A compound according to any one of claims 1, 6, or 8 selected from the group consisting of:

| Compound | |
|---|---|
| No. | Compound Name |
| 1 | (5Z)-3-[(1-methanesulfonylpyrrolidin-3-yl)methyl]-5-[(2,4,6-trifluoro-3- |
| | hydroxyphenyl)methylidene]-1,3-thiazolidine-2,4-dione |
| 2 | (5Z)-3-[(3,4-difluorophenyl)methyl]-5-[(4-fluoro-3-hydroxyphenyl)methylidene]- |
| | 1,3-thiazolidine-2,4-dione |
| 3 | (5Z)-3-[(3-hydroxyphenyl)methyl]-5-[(2,4,5-trifluoro-3- |
| | hydroxyphenyl)methylidene]-1,3-thiazolidine-2,4-dione |
| 4 | (5Z)-3-[(3-hydroxyphenyl)methyl]-5-[(2,4,6-trifluoro-3- |
| | hydroxyphenyl)methylidene]-1,3-thiazolidine-2,4-dione |
| 5 | (5Z)-3-[(3-methanesulfonylphenyl)methyl]-5-[(2,4,6-trifluoro-3- |
| | hydroxyphenyl)methylidene]-1,3-thiazolidine-2,4-dione |
| 6 | (5Z)-5-[(2,4-difluoro-3-hydroxyphenyl)methylidene]-3-[(3- |
| | hydroxyphenyl)methyl]-1,3-thiazolidine-2,4-dione |
| 7 | (5Z)-5-[(3,4-difluoro-5-hydroxyphenyl)methylidene]-3-[(3- |
| | hydroxyphenyl)methyl]-1,3-thiazolidine-2,4-dione |
| 8 | (5Z)-5-[(4-fluoro-3-hydroxyphenyl)methylidene]-3-[(2-fluoro-5- |
| | hydroxyphenyl)methyl]-1,3-thiazolidine-2,4-dione |
| 9 | (5Z)-5-[(4-fluoro-3-hydroxyphenyl)methylidene]-3-[(3-fluoro-5- |
| | hydroxyphenyl)methyl]-1,3-thiazolidine-2,4-dione |
| 10 | (5Z)-5-[(4-fluoro-3-hydroxyphenyl)methylidene]-3-[(3-hydroxyphenyl)methyl]- |
| | 1,3-oxazolidine-2,4-dione |
| 11 | (5Z)-5-[(4-fluoro-3-hydroxyphenyl)methylidene]-3-[(3-hydroxyphenyl)methyl]- |
| | 1,3-thiazolidine-2,4-dione |
| 12 | (5Z)-5-[(4-fluoro-3-hydroxyphenyl)methylidene]-3-[(3- |
| | methanesulfonylphenyl)methyl]-1,3-thiazolidine-2,4-dione |
| 13 | (5Z)-5-[(4-fluoro-3-hydroxyphenyl)methylidene]-3-[(oxan-4-yl)methyl]-1,3- |
| | oxazolidine-2,4-dione |
| 14 | (5Z)-5-[(4-fluoro-3-hydroxyphenyl)methylidene]-3-[(oxan-4-yl)methyl]-1,3- |
| | thiazolidine-2,4-dione |
| 15 | (5Z)-5-[(4-fluoro-3-hydroxyphenyl)methylidene]-3-{[3-(1H-1,2,3-triazol-4- |
| | yl)phenyl]methyl}-1,3-thiazolidine-2,4-dione |
| 16 | (5Z)-5-[(4-fluoro-3-hydroxyphenyl)methylidene]-3-{[3- |
| | (trifluoromethoxy)phenyl]methyl}-1,3-thiazolidine-2,4-dione |
| 17 | (5Z)-5-[(4-fluoro-3-hydroxyphenyl)methylidene]-3-{[3- |
| | (trifluoromethyl)phenyl]methyl}-1,3-thiazolidine-2,4-dione |
| 18 | 3-{[(5Z)-5-[(4-fluoro-3-hydroxyphenyl)methylidene]-2,4-dioxo-1,3-thiazolidin-3- |
| | yl]methyl}benzamide |
| 19 | 3-{[(5Z)-5-[(4-fluoro-3-hydroxyphenyl)methylidene]-2,4-dioxo-1,3-thiazolidin-3- |
| | yl]methyl}benzonitrile |
| 20 | N-(3-{[(5Z)-5-[(4-fluoro-3-hydroxyphenyl)methylidene]-2,4-dioxo-1,3-thiazolidin- |
| | 3-yl]methyl}phenyl)methanesulfonamide |
| 21 | (Z)-5-(4-fluoro-3-hydroxybenzylidene)-3-(3-nitrobenzyl)thiazolidine-2,4-dione |
| 22 | (5Z)-3-[(2-methoxypyridin-4-yl)methyl]-5-[(2,4,6-trifluoro-3- |
| | hydroxyphenyl)methylidene]-1,3-thiazolidine-2,4-dione |
| 23 | (5Z)-3-[(6-methoxypyridin-3-yl)methyl]-5-[(2,4,6-trifluoro-3- |
| | hydroxyphenyl)methylidene]-1,3-thiazolidine-2,4-dione |
| 24 | 6-fluoro-7-hydroxy-N-[(3-hydroxyphenyl)methyl]quinoline-2-carboxamide |
| 25 | 6-fluoro-7-hydroxy-N-{[3-(1H-1,2,3,4-tetrazol-5-y])phenyl]methyl}quinoline-2- |
| | carboxamide |
| 26 | 7-fluoro-6-hydroxy-N-[(3-hydroxyphenyl)methyl]isoquinoline-3-carboxamide |
| 27 | N-[(3-cyanophenyl)methyl]-6-fluoro-7-hydroxyquinoline-2-carboxamide |
| 28 | 2,6-difluoro-3-[5-(4-fluoro-3-hydroxybenzoyl)thiophen-2-yl]phenol |
| 29 | 3-[5-(2,4-difluoro-3-hydroxyphenyl)thiophene-2-carbonyl]-2,4,6-trifluorophenol |
| 30 | 3-[5-(3,4-difluoro-5-hydroxybenzoyl)thiophen-2-yl]-2,6-difluorophenol |
| 31 | 3-[5-(2,4-difluoro-3-hydroxyphenyl)thiophene-2-carbonyl]-2,5,6-trifluorophenol |
| 32 | 3-[5-(2,4-difluoro-3-hydroxybenzoyl)thiophen-2-yl]-2,6-difluorophenol |
| 33 | 3-{[(5Z)-5-[(4-fluoro-3-hydroxyphenyl)methylidene]-2,4-dioxo-1,3-thiazolidin-3- |
| | yl]methyl}benzene-1-sulfonamide |
| 34 | 3-{[(5Z)-2,4-dioxo-5-[(2,4,6-trifluoro-3-hydroxyphenyl)methylidene]-1,3- |
| | thiazolidin-3-yl]methyl}benzene-1-sulfonamide |
| 35 | (5Z)-3-[(6-oxo-1,6-dihydropyridin-3-yl)methyl]-5-[(2,4,6-trifluoro-3- |
| | hydroxyphenyl)methylidene]-1,3-thiazolidine-2,4-dione |
| 36 | (5Z)-3-[(2-oxo-1,2-dihydropyridin-4-yl)methyl]-5-[(2,4,6-trifluoro-3- |
| | hydroxyphenyl)methylidene]-1,3-thiazolidine-2,4-dione |
| 37 | (5Z)-5-[(2,6-difluoro-3-hydroxyphenyl)methylidene]-3-[(3- |
| | hydroxyphenyl)methyl]-1,3-thiazolidine-2,4-dione |
| 38 | (5Z)-5-[(1H-1,2,3-benzotriazol-6-yl)methylidene]-3-[(3-hydroxyphenyl)methyl]- |
| | 1,3-thiazolidine-2,4-dione |
| 39 | 6,8-difluoro-7-hydroxy-N-[(3-methoxyphenyl)methyl]quinoline-2-carboxamide. |

13. A pharmaceutical composition comprising a compound of any one of claims 1-12.

14. The composition of claim 13, further comprising a pharmaceutically acceptable carrier or diluent.

15. The composition of claim 13, further comprising at least one additional therapeutic agent.

16. The composition of claim 15, wherein the at least one additional therapeutic agent is selected from selonsertib, cenicriviroc, elafibrinor, obeticholic acid, or any combination thereof.

17. The composition of claim 13, comprising no or substantially no amount of any additional therapeutic agent.

18. The composition of claim 15, wherein the additional therapeutic agent is selected from the group consisting of an farnesoid X receptor (FXR) activator, an acetyl-CoA carboxylase (ACC) inhibitor, an apoptosis signal-regulating kinase 1 (ASK1) inhibitor, PPAR alpha delta agonist, FGF19 agonist, thyroid beta agonist, FGF21 analog, ACC inhibitors, and a PNPLA3 modulator.

19. The composition of claim 18, wherein the PNPLA3 modulator is selected from the group consisting of an antagonist, an inhibitor, a protein degrader, an RNA interference molecule, or an antisense oligonucleotide.

20. The composition of claim 15, wherein the at least one additional therapeutic agent is selected from the group consisting of selonsertib, cenicriviroc, elafibrinor, obeticholic acid, firsocostat, and ciofexor.

21. A pharmaceutical composition for the treatment or prevention of a condition comprising a compound of any one of claims 1-12, or a pharmaceutically acceptable salt, ester, or amino acid conjugate thereof.

22. The pharmaceutical composition of claim 21, wherein the condition is selected from primary sclerosing cholangitis, chronic liver disease, nonalcoholic fatty liver disease (NAFLD), nonalcoholic steatohepatitis (NASH), hepatitis C, alcoholic liver disease, liver damage due to progressive fibrosis, and liver fibrosis.

23. A method for the treatment or prevention of a condition comprising administering to a subject in need thereof a therapeutically effective amount of a composition comprising the compound of any one of claims 1-12, or combination thereof.

24. The method of claim 23, wherein the condition is selected from primary sclerosing cholangitis, chronic liver disease, nonalcoholic fatty liver disease (NAFLD), nonalcoholic steatohepatitis (NASH), hepatitis C, alcoholic liver disease, liver damage due to progressive fibrosis, and liver fibrosis.

25. The method of claim 23, wherein the condition is nonalcoholic steatohepatitis (NASH).

26. The method of claim 23, wherein the composition is administered to the subject twice daily, once daily, once every other day, or once weekly.

27. The method of claim 23, wherein the composition comprises about 1 mg to about 2000 mg of the compound.

28. The method of claim 23, wherein the subject exhibits at least one of: (a) at least 1 stage improvement in fibrosis without worsening of NASH; (b) reduction of resolution of NASH without worsening of NASH; (c) event-free survival (EFS); (d) cirrhosis-free survival; (e) reduction in complications of ascites; (f) transplant-free survival; (g) variceal hemorrhage-free survival; (h) hepatic encephalopathy-free survival; (i) improvement in Model For End-Stage Liver Disease (MELD) score; and (j) survival.

29. The method of claim 24, wherein the subject exhibits at least one of: (a) improvement of fibrosis by at least 1 stage and/or resolution of NASH, without worsening of either; (b) no worsening of fibrosis and no worsening of NASH; (c) improvement in each histological feature of NASH by at least 1 point; (d) improvement of fibrosis by at least 2 stages; (e) improvement in NASH by at least 2 points with no worsening of fibrosis; (f) NASH resolution with no worsening of fibrosis; (g) improvement of fibrosis and resolution of NASH as a composite endpoint and as defined by both endpoints being met in the same subject; (h) resolution of fibrosis; or (i) no histological progression to cirrhosis.

30. The method of claim 23, wherein the subject exhibits at least one stage, at least two stages, at least three stages, or more of liver fibrosis improvement.

31. The method of claim 30, wherein the improvement is determined histologically.

32. The method of claim 30, further comprising no worsening of NASH.

33. The method of claim 30, further comprising an improvement of NASH.

34. The method of claim 23, wherein the subject exhibits NASH resolution.

35. The method of claim 34, further comprising no worsening of liver fibrosis.

36. The method of claim 30, wherein the improvement is statistically significant.

37. The method of claim 36, wherein the improvement is compared to a control.

38. The method of claim 23, wherein the subject does not experience an increase in pruritis.

39. The method of claim 23, wherein the subject does not experience a statistically significant increase in plasma triglycerides, LDL, or cholesterol.

40. The method of claim 23, wherein the subject experiences hepatic histological improvement in NASH by greater than or equal to 2 points with at least 1-point reduction in either lobular inflammation or hepatocellular ballooning and no concurrent worsening of fibrosis.

41. The method of claim 23, wherein the subject experiences a decrease in hepatic fat.

42. The method of claim 23, wherein the subject experiences an improvement in serum ALT, AST, and/or GGT.

43. The method of claim 23, wherein the subject experiences a reduced in markers of bile acid synthesis.

44. The method as in any one of claims 30, 33, 36, 37, or 42, wherein the improvement occurs within about 4 weeks, about 8 weeks, about 12 weeks, about 24 weeks, or about 48 weeks from first administration of the composition.

45. The method of claim 23, wherein the subject was not responsive to a prior treatment with selonsertib, cenicriviroc, elafibrinor, obeticholic acid, or any combination thereof.

46. The method of claim 23, wherein the subject is a mammal.

47. The method of claim 46, wherein the mammal is a human.

48. The method of claim 23, wherein the subject exhibits an improvement in one or more of a metabolic syndrome, an adverse cardiovascular event, or diabetes mellitus.

49. The method of claim 48, wherein the metabolic syndrome is selected from waist circumference, obesity, hyperglycemia, dyslipidemia, and systemic hypertension (HTN).

50. The method of claim 23, wherein the subject exhibits no or substantially no weight gain, fluid retention, osteopenia, or increased fracture risk.

51. The method of claim 23, wherein the subject is heterozygous or homozygous for the PNPLA3 p.I148M allele (rs738409:G).

52. The method of claim 23, wherein the subject has high polygenic risk for cirrhosis.

53. The method of claim 23, wherein the subject has high polygenic risk for decompensated cirrhosis.

54. The method of claim 53, wherein the decompensated cirrhosis is jaundice, ascites, spontaneous bacterial peritonitis, variceal hemorrhage, hepatic encephalopathy, or hepatorenal syndrome.

55. The method of claim 23, wherein the subject has high polygenic risk for one or more of liver transplant, hepatocellular carcinoma, liver-related mortality, and end stage liver disease.

56. The method of claim 55, wherein the end stage liver disease is selected from the group consisting of decompensated cirrhosis, liver transplant, hepatopulmonary syndrome, complicated portal hypertension, hepatocellular carcinoma, or liver-related mortality.
